(19) United States
(12) Patent Application Publication
(10) Pub. No.: US 2026/0325485 A1
(43) Pub. Date: Oct. 22, 2026

(54) COMPOSITIONS AND METHODS FOR TREATING PANCREATITIS

(71) Applicant: The United States Government as represented by the Department of Veterans Affairs, Washington, DC (US)

(72) Inventors: Anna Gukovskaya, Los Angeles, CA (US); Ilya Gukovsky, Los Angeles, CA (US)

(21) Appl. No.: 19/100,559

(22) PCT Filed: Aug. 2, 2023
(86) PCT No.: PCT/US2023/029332

§ 371 Date: Jan. 31, 2025

Related U.S. Application Data
(60) Provisional application No. 63/394,444, filed on Aug. 2, 2022.

(51) Int. Cl. A61K 31/366; A61K 31/20; A61K 45/06; A61P 1/18; A61P 3/06

(52) U.S. Cl. CPC A61K 31/366; A61K 31/20; A61K 45/06; A61P 1/18; A61P 3/06

(57) ABSTRACT

Disclosed are methods of treating pancreatitis in a subject comprising administering to the subject a therapeutically effective amount of a cholesterol lowering drug. Disclosed are methods of reducing acinar cell cholesterol in a subject comprising administering to the subject a therapeutically effective amount of a cholesterol lowering drug. Disclosed are methods of reducing necrosis in the pancreas of a subject comprising administering to the subject a therapeutically effective amount of a cholesterol lowering drug. In some aspects, the cholesterol lowering drug can be a statin or adenosine triphosphate citrate lyase inhibitor.

### CROSS-REFERENCE TO RELATED APPLICATIONS

This application claims the benefit of U.S. Provisional Patent Application No. 63/394,444, filed Aug. 2, 2022, incorporated by reference herein in its entirety.

### STATEMENT REGARDING FEDERALLY SPONSORED RESEARCH

This invention was made with government support under Grant Numbers W81XWH1910888 awarded by the US Department of Defense, P50 AA11999 awarded by the National Institute of Health, and BX004306 awarded by Veterans Affair. The government has certain rights in this invention.

### BACKGROUND

Pancreatitis is a progressive, potentially fatal, disease of the exocrine pancreas characterized by inflammation, fibrosis, and loss of pancreas functions. There are two major forms of pancreatitis, acute (AP), which usually produces an episode of temporary illness, and chronic (CP), a disease associated with severe pain, poor quality of life, and increased risk for the deadliest pancreatic cancer. CP may result from repetitive subclinical or clinically evident bouts of AP or may develop without prior AP. Pancreatitis is the third most common reason for hospital admissions in those with GI disease and a heavy burden on the U.S. healthcare system. Major AP responses include inappropriate/intra-acinar activation of digestive enzymes, particularly trypsinogen; increased serum levels of amylase; neutrophil-driven inflammatory cell infiltration in the pancreas; and acinar cell death. Key pathologic features of CP are progressive loss of acinar tissue, chronic inflammation, and fibrosis, ultimately leading to loss of exocrine as well as endocrine pancreatic function. The disease may be initiated by injured acinar cells; there is compelling evidence that mechanisms mediating pancreatitis are common to all forms of the disease. The pathogenesis of pancreatitis remains obscure, and no effective treatment is available.

### BRIEF SUMMARY

Disclosed are methods of treating pancreatitis in a subject comprising administering to the subject a therapeutically effective amount of a cholesterol lowering drug.

Disclosed are methods of reducing acinar cell cholesterol in a subject comprising administering to the subject a therapeutically effective amount of a cholesterol lowering drug.

Disclosed are methods of reducing necrosis in the pancreas of a subject comprising administering to the subject a therapeutically effective amount of a cholesterol lowering drug.

In some aspects, the cholesterol lowering drug can be a statin or an adenosine triphosphate citrate lyase inhibitor.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-IN show Gnptab ablation perturbs the endolysosomal system in the exocrine pancreas. Characteristics of the endolysosomal system were measured in WT and Gnptab−/−(KO) pancreatic (A, B, D, and E) tissue, (C) acinar cells, and (F-N) subcellular fractions. (A and D-F) IB analysis of organellar markers/mediators. In this and other figures, ERK1/2 or GAPDH is the loading control; each lane on tissue IB represents an individual mouse; black line (as in F) indicates lanes are on the same gel but not contiguous. Ub, ubiquitylated proteins. (B) Immunofluorescence analysis of indicated proteins. In this and other figures, nuclei are stained with DAPI (or NucRed); DIC denotes differential interference contrast microscopy, displaying zymogen granule area in acinar cells. Scale bars: 10 μm. (C) Acidic organelles stained using LysoTracker. (D and E) IB showing CatB and CatL pro-form, single-chain (sc), and mature/double-chain (dc) forms. (F-N) Protein levels of indicated markers/mediators (IB) and cathepsin activities (enzymatic assays) were measured in 3 subcellular pancreas fractions (see Methods): Z, enriched in zymogen granules; L, enriched in LE/Ly (in WT); and C, cytosol containing. The sum of band intensities for indicated proteins or cathepsin activities in all 3 fractions was taken as 100%. In F, # indicates that protein load of KO samples was 5 times less than WT. Values are mean±SEM; each symbol represents an individual mouse (n=3-6 mice per condition). *P<0.05, **P<0.01, ***P<0.001 vs. WT tissue (D and E) or corresponding WT fraction (G-N). #P<0.01 (G and H) or †P<0.001 (J and K) vs. the other two fractions. Significance was determined by 2-tailed Student's t test (D and E) or 1-way ANOVA followed by Tukey's multiple comparison test (G-N).

FIGS. 2A-2L show Gnptab ablation stimulates autophagosome formation and blocks autophagic flux in pancreatic acinar cells. Parameters of autophagy were measured in WT and Gnptab+ (KO) pancreatic (A-C) acinar cells and (D-L) tissue. (A-C) Acinar cells incubated for 30 minutes with and without bafilomycin A1 (BafA1; 20 nM); LC3-II band intensity was quantified by densitometry, normalized to ERK in the same sample, and LC3-II/ERK ratios further normalized to untreated WT cells (control). Values are mean±SEM; each symbol represents an individual cell preparation (n=4-6 per condition). sP<0.05; paired 2-tailed Student's t test. (D) IB analysis of autophagic markers/mediators. Ub, ubiquitylated proteins. (E-K) Immunofluorescence colocalization analysis for LC3 and LAMP1 (E, G, and H), and LC3 and Stx17 (F and I-K) in WT and KO pancreas. In this and other figures, colocalization was quantified with Volocity software using Manders-Costes (percentage colocalization) and Pearson's coefficients. Values are mean±SEM; each symbol corresponds to 20 to 30 cells in a different field (n=6-9 fields from 3 mice per condition). ***P<0.001 vs. WT; 2-tailed Student's t test. (L). EM showing abnormally large autolysosomes with partially degraded material in pancreas of KO mice. Scale bars: 10 μm (E and F) and 3 μm (L).

FIGS. 3A-3L show Gnptab ablation impairs early-to-late endosome maturation and endocytic recycling. Immunofluorescence analysis of markers of early (EEA1, TfR, Rab5), recycling (Rab11), and late (LAMP2, Rab7) endosomes in pancreatic (A and C—H) tissue and (B, I, and J) acinar cells (see also images in the FIG. 12). In I and J, acinar cells were transduced with both CellLight Rab5-GFP and Rab7-RFP. Quantitative colocalization analysis for indicated proteins was done as detailed in FIGS. 2A-2L. Values are mean±SEM; each symbol corresponds to 20 to 30 cells in a different field (n=7-14 fields from 3 to 4 mice or cell preparations per condition). ***P<0.001 vs. WT; 2-tailed Student's t test. (K and L) To measure transferrin (Tf) endocytosis, acinar cells were loaded with Tf-594 at 0° C., transferred to 37° C., incubated for the indicated times, and visualized with confocal microscopy. Internalized Tf-594 was quantified using Volocity software. Values are mean±SEM; each symbol corresponds to 10 to 15 cells in a different field (n=6-11 fields from 3 WT and 3 KO cell preparations). ***P<0.001 vs. corresponding WT condition; 1-way ANOVA followed by Tukey's multiple comparison test. Scale bars: 10 μm.

FIGS. 4A-4R show Gnptab ablation disrupts cholesterol metabolism in the exocrine pancreas. Parameters of cholesterol metabolism were measured in pancreatic (A-H) tissue and (I-R) acinar cells. (A-D) Pancreatic contents of total and nonesterified (free) cholesterol, triglycerides, and free fatty acids (FFA); each symbol represents an individual mouse. (E) Heatmap of lipid species of indicated cholesterol esters (CE), phosphatidylcholine (PC), phosphatidylethanolamine (PE), and phosphatidylserine (PS), measured with LC-MS/GC-MS. Each row represents an individual mouse. (F) mRNA expression of proteins regulating the synthesis of cholesterol and fatty acids was measured by qPCR; each symbol represents an individual mouse. Values were normalized to the housekeeping gene Rplp0/36B4 and are expressed relative to WT. (G) Lysosomal acid lipase (LAL) activity was measured with a fluorogenic assay. (H) IB analysis of NPC1, NPC2, and LDLR. (I) LDL uptake was measured with DyLight550-LDL probe; each symbol represents a different cell preparation. (J-R) Colocalization of the free-cholesterol probe filipin with indicated proteins (see also images in FIGS. 15 and 16) was quantified using confocal microscopy and the Manders-Costes coefficient. Each symbol corresponds to 10 to 15 cells in a different field (n=5-11 fields from 3 WT and 3 KO cell preparations). Values are mean±SEM from 3 to 7 (A and C-G) or 14 to 15 (B) mice of each strain, or from 3 to 4 acinar cell preparations per condition (I and J-R). **P<0.01, ***P<0.001 vs. WT; 2-tailed Student's t test. Scale bars: 10 μm.

FIGS. 5A-5M show Gnptab^{-/-} mice develop pancreatitis. Parameters of pancreas (A-K) and liver (L and M) damage in KO mice of different age (3 months old [mo] if not specified). (A and L) Histopathologic changes (H&E staining). (B-F) Pancreatitis responses. In F, macrophage (F4/80), neutrophil (MPO; myeloperoxidase), and T cell (CD3) infiltration in pancreas was quantified by IHC (see images in FIG. 19). (G and J) IB analysis of total and phosphorylated (active) NF-κB (p-p65/RelA) and STAT3 (p-Tyr705-STAT3), and other proteins in pancreas. (H) Proinflammatory cytokines and chemokines were measured (per mg protein) with Luminex Multiplexing analyzer. (I) The basal/unstimulated amylase secretion was measured as a percentage of its total acinar cell content. (K) Sirius red staining showing pancreatic fibrosis. (M) Serum markers of liver injury. Values are mean±SEM from 3 to 8 mice of each strain; each symbol represents an individual mouse or cell preparation. *P<0.05, **P<0.01, ***P<0.001 vs. WT; 2-tailed Student's t test. Scale bars: 10 μm.

FIGS. 6A-6R show mechanisms mediating increased trypsin activity in Gnptab−/− pancreas. Trypsin activity and its regulation were analyzed in pancreatic (A, C, and D) tissue, (B and P-R) acinar cells, (E-H and O) lobules, and (I-N) subcellular fractions. In B, acinar cells were incubated for 3 hours without and with 20 nM BafAl or 50 μM CatB inhibitor CA-074Me; in P-R, with the cholesterol chelator methyl-β-cyclodextrin (MCD) at the indicated concentrations. In E-H, pancreatic lobules were double stained for TAP and LC3 or LAMP2; in O, for TAP and filipin. Quantitative colocalization analysis was done as detailed in FIG. 2. Each symbol in A, B, J-N, Q, and R represents an individual mouse or cell preparation (n=3-8 per condition). Each symbol in D, G, and H corresponds to 20 to 30 cells in a different field (n=9-20 fields from 3 WT and 3 KO mice or lobule preparations). Values are mean±SEM. **P<0.01, ***P<0.001 vs. WT tissue/lobules (A, G, and H) or corresponding WT subcellular fraction (K and M). ##P<0.01 vs. no inhibitors (B) or MCD (Q and R). ††P<0.01, †††P<0.001 vs. the other two fractions (L and N). Significance was determined by 2-tailed Student's t test (A, G, and H) or 1-way ANOVA followed by Tukey's multiple comparison test (B, K-N, Q, and R). Scale bars: 10 μm.

FIGS. 7A-7Q show Gnptab ablation causes mitochondrial dysfunction in the pancreas. Mitochondrial functional parameters were measured in mitochondria isolated from pancreas (A, D, J, and M) or liver (N-P), in acinar cells (B, C, and I), and pancreatic (E-H, K, and L) and liver (Q) tissues of WT and KO mice. (A and N) Activities of mitochondrial ETC complexes I, II, IV, and V. (B, C, and I) Acinar cell oxygen consumption rates (OCR), reflecting mitochondrial OXPHOS, were measured in basal conditions and after treatment with the B-oxidation inhibitor etomoxir (40 μM), and normalized to total protein in the sample. (B) Representative OCR tracings in acinar cell suspension subjected to consecutive injections of the ATP synthase inhibitor oligomycin (Oligo; 3 μM), protonophore FCCP (2.7 μM), and mitochondrial uncouplers rotenone plus antimycin A (R+A, 5 μM each; to correct for nonmitochondrial respiration). (C and I) ATP-linked respiration was calculated as the difference between basal OCR and that in Oligo-treated cells; the maximal OCR was obtained in FCCP-treated cells. (D and O) Mitochondrial membrane potential (ΔΨm) was measured with a tetraphenyl phosphonium ion (TPP+) electrode. FCCP (5 μM) was added to completely dissipate ΔΨm. (E) ATP was measured in pancreatic tissue homogenate with luciferin-luciferase assay. (F, H, L, and Q) IB analysis of the mitochondrial fission factor Fisl and mitophagy mediator Parkin (F), mitochondrial markers (H), and MLN64/STARD3 protein (L and Q) that mediates cholesterol transfer from LE/Ly directly to mitochondria. (G) Mitochondrial fragmentation was assessed by changes in Tom20 or VDAC immunostaining pattern, from an interconnected tubular mitochondrial network in WT to a predominantly disjoined/circular one in KO pancreas. Scale bars: 10 μm. (J, M, and P) Free fatty acid (FFA) and cholesterol contents of isolated pancreatic or liver mitochondria. (K) Long-chain acylcarnitines were measured with LC-MS/GC-MS. Values are mean±SEM; each symbol represents an individual mouse, or cell or mitochondria preparation (n=3-8 per condition). *P<0.05, **P<0.01, ***P<0.001 vs. the same parameter in WT. #P<0.05 vs. the same condition without etomoxir (I). Significance was determined by 2-tailed Student's t test.

FIGS. 8A-8M show derangement of cholesterol metabolism in experimental pancreatitis. Characteristics of cholesterol metabolism were measured, as detailed in FIGS. 4A-4R, in pancreas of WT mice subjected to pancreatitis induced with cerulein (CER-AP), L-arginine (Arg-AP), or CDE diet (CDE-AP) (A, B, and E-G); ex vivo pancreatitis induced in WT mouse (C and I-M) or human acinar cells (D) with 100 nM CCK (CCK-AP) or 1 mM carbachol; and isolated pancreatic mitochondria (H). (B) Cholesterol ester (CE) heatmap; each column represents an individual mouse. (F) IB analysis of the indicated proteins. (G) LAL activity was measured by a fluorogenic assay (n=6 mice per condition). (H) Cholesterol content was measured by enzymatic assay. (I-M) Filipin colocalization with indicated proteins was quantified as detailed in FIGS. 2A-2L. Each symbol in A, C-E, and H represents an individual mouse, or cell or mitochondria preparation (n=3-15 per condition). Each symbol in J, K, and M corresponds to 20-30 cells in a different field (n=10-20 fields from 3 cell preparations per condition). Values are mean±SEM. *P<0.05, **P<0.01, ***P<0.001 vs. the same parameter in control/untreated pancreas, acinar cells or isolated mitochondria. Significance was determined by 2-tailed Student's t test (B, E, H, J, K, and M) or 1-way ANOVA followed by Tukey's multiple comparison test (A, C, and D). Scale bars: 10 μm.

FIGS. 9A-9E show cerulein pancreatitis decreases pancreatic levels of GNPTAB and mediators of cholesterol trafficking through LE/Ly. (A, D, and E) IB analysis of the indicated proteins in pancreas of WT mice subjected to CER-AP and in Gnptab^{-/-} mice. NS, nonspecific band. (B and C) Cholesterol content was measured in pancreas of (B) WT mice that received daily i.p. injections of the NPC1 inhibitor U18666A (10 mg/kg) or vehicle for 15 days and (C) Lamp2^{-/-} mice. Each symbol in B, C, and E represents an individual mouse. Values are mean±SEM from 3 to 5 mice per condition. ***P<0.001 vs. the same parameter in WT control/untreated or saline-treated mice. Significance was determined by 2-tailed Student's t test (B and C) or 1-way ANOVA followed by Tukey's multiple comparison test (E).

FIGS. 10A-10N show deranged cholesterol metabolism causes acinar cell damage, while its normalization ameliorates experimental pancreatitis. (A-D) Cholesterol content and trypsin activity were measured (per mg protein) in WT mouse acinar cells incubated for 1 hour without and with methyl-β-cyclodextrin (MCD; 10 mM) or cholesterol-loaded MCD (MCD-Chol; 200 μg/mL), in the presence or absence of 100 nM CCK. Values are expressed relative to those in cells without MCD/MCD-Chol. (E-J) WT mice received daily i.p. injections of the LXR agonist T0901317 (T09), simvastatin (Sim; 20 mg/kg each), or vehicle. On day 4, mice were subjected to CER-AP and pancreata collected for analyses (see also FIG. 20). Cholesterol content (E), trypsin activity (F), and pancreas histopathology (H&E staining; G) were measured. (H, I, L, and N) IB analysis of markers/mediators of autophagy (LC3, p62), ER stress (CHOP, GRP-78), and mitophagy (Parkin). Ub, ubiquitylated proteins. (J) Mitochondrial fragmentation was assessed by immunostaining for Tom20, as detailed in FIG. 7G. (K and L) Mouse acinar cells were treated for 16 hours with LDL (30 μg/mL) or the combination of LDL and 10 μM U18666A. (M and N) Mice received injections of U18666A as detailed in FIG. 9B. Pancreas histopathology (H&E staining; M) and levels of autophagy markers/mediators (N) were measured. Each symbol in A-F and I represents an individual mouse or cell preparation. Values are mean±SEM from 3 to 7 mice or cell preparations per condition. ^{§} P<0.05, ^{§§} P<0.01 vs. control cells (without MCD/MCD-Chol). ***P<0.001 vs. saline-treated mice. ^{#}P<0.05, ^{##}P<0.01, ^{###}P<0.001 vs. CER-AP alone (no inhibitors). Significance was determined by 2-tailed Student's t test (A-D) or 1-way ANOVA followed by Tukey's multiple comparison test (E, F, and I). Scale bars: 10 μm.

FIGS. 11A-11F show manifestations of disordered cholesterol homeostasis in human pancreatitis. (A-F) Immunofluorescence intensity of indicated proteins in human pancreatic tissue sections was quantified with Volocity software, normalized to the number of nuclei (DAPI) in the field, and is expressed relative to that in normal pancreas. Each panel in A-C is from a different patient (from a total of 21 patients; see Table 1). Scale bars: 10 μm. (D-F) Values are mean±SEM; each symbol corresponds to 20 to 30 cells in a different field (n=10-23 fields from 10 without pancreatitis and 11 pancreatitis patients). ***P<0.001 vs. normal human pancreas (no pancreatitis); 2-tailed Student's t test.

FIG. 12 shows immunofluorescence colocalization of markers of early (TfR) or recycling (Rab11) endosomes with the LE/Ly marker LAMP2 in Gnptab−/− pancreas. Quantitative analysis of these images is presented in FIGS. 3E-H.

FIGS. 13A-13G show neutral lipids accumulate in LE/Ly and autophagic compartments of Gnptab^{-/-} pancreas. Characteristics of neutral lipid metabolism were measured in (A-C,F,G) pancreatic tissue of WT, Gnptab^{-/-} (KO) and Atg5^{Δpan} mice, and (D,E) WT and KO acinar cells treated with Bafilomycin A1 (BafA1; 20 nM). (A) EM showing lipid inclusions in KO pancreas. (B-E) Tissue or cells were stained with BODIPY 493/503 for neutral lipids; BODIPY fluorescence intensity was quantified with Volocity software, normalized to the number of nuclei (DAPI) in the field, and expressed relative to WT control. In (C), each symbol corresponds to 20-30 cells in a different field (n=5-6 fields from 3 mice of each strain). In (E), each symbol corresponds to 10-15 cells in a different field (n=6-7 fields from 3 WT and 3 KO cell preparations). (F,G) Perilipins (PLINs) were analyzed by immunoblot; their band intensities were densitometrically quantified, normalized to GAPDH in the same sample and, further, to WT. Each symbol represents an individual mouse (n=3 mice of each strain). Each lane on tissue IB represents an individual mouse; ERK1/2 or GAPDH serve as loading control. Values are mean±SEM. *p<0.05, **p<0.01, ***p<0.001 vs the same parameter in WT tissue or (E) control/untreated WT cells. Significance was determined by 2-tailed Student's t test (G) or 1-way ANOVA followed by Tukey's multiple comparisons test (C,E). Scale bars: 5 μm (A) and 10 μm (B,D).

FIGS. 14A-14F show colocalization of neutral lipids stain with markers of autophagic (LC3 puncta) and LE/Ly (LAMP1) compartments in Gnptab^{-/-} pancreas. WT and KO pancreatic tissue sections were stained with BODIPY 493/503 for neutral lipids. Colocalization of BODIPY with LC3 (A-C) or LAMP1 (D-F) was quantified with Volocity software using Manders-Costes coefficients. Each symbol corresponds to 20-30 cells in a different field (n=6-10 fields from 3 mice of each strain). Values are mean±SEM. ***p<0.001 vs the same parameter in WT tissue; 2-tailed Student's t test. Scale bars: 10 μm.

FIGS. 15A-15B show Gnptab ablation depletes non-esterified (free) cholesterol in acinar cell plasma membrane and zymogen granules. Colocalization of the free-cholesterol probe filipin with markers of (A) plasma membrane (cadherin; pan-cadherin Ab) and (B) zymogen granules (amylase) in acinar cells was visualized with confocal microscopy. Quantitative analysis of these images is presented in FIGS. 4J-L. Scale bars: 10 μm.

FIGS. 16A-16B Gnptab ablation perturbs non-esterified (free) cholesterol localization in acinar cell endolysosomal system. Filipin colocalization with markers of (A) LE/Ly (NPC1) and (B) early endosomes (EEA1) in acinar cells was visualized with confocal microscopy. Quantitative analysis of these images is presented in FIGS. 4M,N. Scale bars: 10 μm.

FIGS. 17A-17J show Gnptab ablation does not perturb the endolysosomal system and does not alter cholesterol content in liver. (A-H,J) IB analysis of markers/mediators of late endosomes/lysosomes (LAMP2, Rab7, CatB), cholesterol trafficking (NPC1, NPC2), autophagy (LC3, p62, EPG5), and lipid droplets (PLINs). In (A), CatB immunoblot shows bands corresponding to the proform, intermediate/single-chain (sc) and mature/double-chain (dc) forms. (B-H,J) Protein band intensities in liver (A) and pancreas (see immunoblots in FIGS. 1, 2, 4, and S2) were densitometrically quantified, normalized to ERK or GAPDH in the same sample, and expressed relative to WT. (I) Triglycerides (TAG), free fatty acids (FFA), total (TC) and free (FC) cholesterol contents in liver were measured by enzymatic assays. Values are mean±SEM from 3-4 mice of each strain; each symbol represents an individual mouse. *p<0.05, **p<0.01, ***p<0.001 vs the same tissue in WT. Significance was determined by 2-tailed Student's t test (F,J) or 1-way ANOVA followed by Tukey's multiple comparisons test (B-E,H).

FIGS. 18A-18B show Gnptab ablation does not increase colocalization of early endosomes and neutral lipids with LE/Ly compartment in liver. Immunolabeling for LAMP2 and EEA1, and BODIPY 493/503 staining for neutral lipids in KO liver. Scale bars: 10 μm.

FIG. 19 shows inflammatory cell infiltration in pancreas of Gnptab−/− mice. Representative IHC staining for markers of macrophages (F4/80), neutrophils (myeloperoxidase; MPO), and T-cells (CD3) in WT and KO pancreas. Quantification of these data is shown in FIG. 5F.

FIGS. 20A-20C show simvastatin improves cerulein pancreatitis. Mice received daily i.p. injections of simvastatin (Sim; 20 mg/kg) or vehicle (see FIG. 10), and on day 4 were subjected to cerulein pancreatitis (CER-AP). (A) Pancreas histopathology (H&E staining; scale: 10 μm). (B) IB analysis of markers/mediators of autophagy (LC3, p62), ER stress (CHOP), and the MLN64/STARD3 protein mediating cholesterol transfer from LE/Ly to mitochondria. Each IB lane represents an individual mouse; ERK1/2 is the loading control. The narrow black line indicates that lanes are on the same gel but not contiguous. Ub, ubiquitylated proteins. (C) Pancreatitis responses. Values are mean±SEM from 7-13 mice for each condition. ***p<0.001 vs saline control. ^{###}p<0.001 vs CER-AP alone (no simvastatin). Significance was determined by 1-way ANOVA followed by Tukey's multiple comparisons test.

FIG. 21 shows routes of free cholesterol production in cells.

FIG. 22 shows inhibitors of cholesterol biosynthesis pathway.

FIGS. 23A-23F show dysregulation of cholesterol homeostasis in pancreatitis.

Parameters of cholesterol metabolism were measured in (A, B, E, F) pancreas of mice subjected to pancreatitis induced with cerulean (CER-AP), L-arginine (Arg-AP), or combination of EtOH+palmitoleic acid (EtOH+POA); and (C) ex-vivo models on mouse and human acinar cells treated for 1 h with 100 nM CCK, 10 mM carbachol or 500 μM TLCS. In (A), each column in CE heatmap represents an individual mouse. (B, C) Total cholesterol content was measured with cholesterol assay kit (Abcam, ab65390). (D) Pancrease tissue sections from patients with and without pancreatitis were stained with the free Ocholesterol prove filipin; fluorescence intensity was quantified and expressed relative to normal pancreas. Each symbol corresponds to 20-30 cells in each of 8 high-expression (qPCR) of proteins regulating cholesterol synthesis (4 mice/group). (B-D) Each symbol represents an individual animal or cell preparation. (A-D, F) Values are mean±SE (*p<0.05, **p<0.01, ***p<0.001 vs saline or control). Signficance was determined by 2-tailed student's t test.

FIGS. 24A-24B show HMGOCoA-reductase upregulation in human pancreas. (A, B) Immunogluorescence intensity of HMGCR in human pancreatic tissue sections was quantified with Volocity software, normalized to the number of nuclei (DAPI) in the field, and is expressed relative to that in normal pancreas. Each panel in (A) is from a different patient. Scale bars: 10 μm. (B) Values are mean±SEM; each symbol corresponds to 20 to 30 cells in a different field (n=10-23 fields from 10 without pancreatitits and 11 pancreatitis patients). ***P<0.001 vs. normal human pancreas (no pancreatitis).

FIGS. 25A-25C show reducing cholesterol overload greatly ameliorates experimental pancreatitis. CER-AP responses were measured in (A) AclyAac or (C) WT mice treated with simvastatin or bempedoic acid (BemA). (B) Acinar cells were transfected with ACLA (or mock) siRNA and subjected to ex-vivo CCK-AP. Each symbol represents an individual mouse or cell preparation. Values are mean±SE. **p<0.01, ***p<0.001 vs saline treated control mice (or mock transfection); #p<0.05, ##p<0.01, ###p<0.001 vs CER/CCK-AP in control mice (i.e. without inhibitors) or cells.

FIGS. 26A-26D preventing cholesterol overload ameliorates ex-vivo pancreatitis. Mouse pancreatic acinar cells were incubated (A, B) with simvastatin (4 μM, 3 h) or (C, D) cholesterol chelator, methyl-β-cyclodextrin (MCD; 10 mM, 1 h) and then subjected to CCK-AP for 1 (D) or 3 h (A, B). Cholesterol content (A, C) necrosis (B), and trypsin activity (D) were measured. Each symbol represents an individual cell preparation. Values are mean±SEM*p<0/05, **p<0.01 vs control; #p<0.05 ##p<0.01; vs CCK, no inhibitor.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cholesterol lowing drug” includes a plurality of such cholesterol lowing drugs, reference to “the cholesterol lowing drug” is a reference to one or more cholesterol lowing drugs and equivalents thereof known to those skilled in the art, and so forth.

By a “therapeutically effective amount” of a composition as provided herein is meant a sufficient amount of the composition to provide the desired therapeutic effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “therapeutically effective amount.” However, an appropriate “therapeutically effective amount” may be determined by one of ordinary skill in the art using only routine experimentation.

“Treatment” and “treating” refer to administration or application of a therapeutic agent (e.g., a peptide or polypeptide described herein) to a subject or performance of a procedure or modality on a subject for the purpose of obtaining a therapeutic benefit of a disease or health-related condition. As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the “treat” can refer to administering a cholesterol lowing drug or composition of the invention to a subject, such as a human or other mammal (for example, an animal model), that has an increased susceptibility for developing pancreatitis, or that has pancreatitis, in order to prevent or delay a worsening of the effects of the disease or condition, or to partially or fully reverse the effects of the disease.

The terms “preventing,” “blocking,” “antagonizing,” or “reversing” mean preventing in whole or in part, or ameliorating or controlling.

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

By “prevent” is meant to minimize the chance that a subject who has an increased susceptibility for developing pancreatitis will end up with pancreatitis.

The terms “patient,” “subject,” “individual,” and the like are used interchangeably herein, and refer to any animal, or cells thereof whether in vitro or in situ, amenable to the methods described herein. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

An “effective amount” of a composition is that amount of composition which is sufficient to provide a beneficial effect to the subject to which the composition is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a composition.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Treating

Disclosed are methods of treating pancreatitis in a subject comprising administering to the subject a therapeutically effective amount of a cholesterol lowering drug. Disclosed are methods of ameliorating a symptom associated with pancreatitis in a subject comprising administering to the subject a therapeutically effective amount of a cholesterol lowering drug. In some aspects, a symptom associated with pancreatitis can include belly pain, nausea, vomiting, rapid heart rate, and jaundice. In some aspects, more than one cholesterol lowering drug can be administered to the subject.

As used herein, a “cholesterol lowering drug” refers to a therapeutic that can reduce or substantially reduce cholesterol levels in a subject. In some aspects, the cholesterol levels are total cholesterol, serum cholesterol or pancreatic cell cholesterol. In some aspects, a cholesterol lowering drug can refer to a therapeutic that can normalize cholesterol metabolism and/or lower serum cholesterol in a subject.

In some aspects, the cholesterol lowering drug is a statin. Statins, also known as HMG-COA reductase inhibitors, are a class of lipid-lowering medications that reduce illness and mortality in those who are at high risk of cardiovascular disease. In some aspects, the statin can be, but is not limited to, atorvastatin, fluvastatin, lovastatin, pitavastatin, pravastatin, rosuvastatin, and simvastatin. In some aspects, the statin is simvastatin.

In some aspects, the cholesterol lowering drug is an adenosine triphosphate citrate lyase (ACLY) inhibitor. In some aspects, the adenosine triphosphate citrate lyase inhibitor can prevent catalyses of ATP-dependent and coenzyme A (CoA)-dependent conversion of citrate, to oxaloacetate and the high-energy biosynthetic precursor acetyl-CoA. In some aspects, the adenosine triphosphate citrate lyase inhibitor can be, but is not limited to, bempedoic acid, SB-204990, NDI-091143, (−)-Hydroxycitric acid, or BMS-303141.

In some aspects, the cholesterol lowering drug is a liver X receptor (LXR) agonist. In some aspects, the LXR agonist can induce the transcriptional activity of LXR target genes. As used herein, the LXR agonist can be a natural and synthetic LXR agonists that can induce the transcriptional activity of LXR target genes, attenuate the imbalance of cholesterol metabolism or overactivation of microglia and astrocytes in inflammation. In some aspects, the LXR agonist is T0901317. In some aspects, the LXR agonist can be an oxysterol. In some aspects, oxysterols can be the oxygenated derivatives of cholesterol, such as, but not limited to, 22(R)-hydroxycholesterol, 24(S)-hydroxycholesterol, 27-hydroxycholesterol, or cholestenoic acid.

In some aspects, the cholesterol lowering drug normalizes cholesterol metabolism. In some aspects, an example of normalizing cholesterol metabolism can be a reduction in total cholesterol, serum cholesterol or pancreatic cholesterol. In some aspects, an example of normalizing cholesterol metabolism can be a reduction in cholesterol to levels present in subjects not having pancreatitis.

Disclosed are any of the methods of treating pancreatitis or ameliorating a symptom of pancreatitis in a subject comprising administering to the subject a therapeutically effective amount of a cholesterol lowering drug and further comprising administering a second pancreatitis therapy to the subject. In some aspects, a second pancreatitis therapy can be treholose, an authophagy enhancer. In some aspects, a second pancreatitis therapy can be sulforaphane, an activator of the protein NRF2. In some aspects, a second pancreatitis therapy can be a pain medication, anti-inflammatoires, vitamins, digestives, or i.v. fluids. In some aspects, the second pancreatitis therapy can be administered before, simultaneously with, or after administering the cholesterol lowering drug.

### C. Methods of Reducing Cholesterol

Disclosed are methods of reducing acinar cell cholesterol in a subject comprising administering to the subject a therapeutically effective amount of a cholesterol lowering drug.

In some aspects, acinar cell cholesterol is reduced 1-fold, 1.5-fold, 2-fold or more when compared to untreated subjects. As used herein, “untreated subject” refers to a subject that was not administered a therapeutically effective amount of a cholesterol lowering drug. When comparing a subject who was administered a therapeutically effective amount of a cholesterol lowering drug to an untreated subject, the subjects can be chosen such that each subject has the same condition or disease (e.g. elevated cholesterol or elevated acinar cell cholesterol). In some aspects, acinar cell cholesterol is reduced at least 10%, 15%, 20%, 25%, 30%, 35%, 40%, 45%, 50% or more when compared to untreated subjects.

In some aspects, the methods of reducing cholesterol can result in a reduction in LC3-II or p62. Thus, disclosed are methods of reducing LC3-II in a subject comprising administering to the subject a therapeutically effective amount of a cholesterol lowering drug. Also disclosed are methods of reducing p62 in a subject comprising administering to the subject a therapeutically effective amount of a cholesterol lowering drug. In some aspects, LC3-II and/or p62 is reduced at least 10% compared to untreated subjects.

### D. Methods of Reducing Necrosis

Disclosed are methods of reducing necrosis in the pancreas of a subject comprising administering to the subject a therapeutically effective amount of a cholesterol lowering drug. In some aspects, the subject has pancreatitis or is susceptible to pancreatitis.

In some aspects, the necrosis is due to pancreatitis. Thus, a reduction in necrosis helps maintain a more viable pancreas. In some aspects, a reduction in necrosis is a decrease in at least 5%, 10%, 15%, 20%, 25%, 30%, 35%, 40%, 45%, or 50% compared to the pancreas of an untreated subject.

In some aspects, the cholesterol lowering drug is a statin. Statins, also known as HMG-COA reductase inhibitors, are a class of lipid-lowering medications that reduce illness and mortality in those who are at high risk of cardiovascular disease. In some aspects, the statin can be, but is not limited to, atorvastatin, fluvastatin, lovastatin, pitavastatin, pravastatin, rosuvastatin, and simvastatin. In some aspects, the statin is simvastatin.

In some aspects, the cholesterol lowering drug is an adenosine triphosphate citrate lyase (ACLY) inhibitor. In some aspects, the adenosine triphosphate citrate lyase inhibitor can prevent catalyses of ATP-dependent and coenzyme A (CoA)-dependent conversion of citrate, to oxaloacetate and the high-energy biosynthetic precursor acetyl-CoA. In some aspects, the adenosine triphosphate citrate lyase inhibitor can be, but is not limited to, bempedoic acid, SB-204990, NDI-091143, (−)-Hydroxycitric acid, or BMS-303141.

In some aspects, the cholesterol lowering drug is a liver X receptor (LXR) agonist. In some aspects, the LXR agonist can induce the transcriptional activity of LXR target genes. As used herein, the LXR agonist can be a natural and synthetic LXR agonists that can induce the transcriptional activity of LXR target genes, attenuate the imbalance of cholesterol metabolism or overactivation of microglia and astrocytes in inflammation. In some aspects, the LXR agonist is T0901317. In some aspects, the LXR agonist can be an oxysterol. In some aspects, oxysterols can be the oxygenated derivatives of cholesterol, such as, but not limited to, 22(R)-hydroxycholesterol, 24(S)-hydroxycholesterol, 27-hydroxycholesterol, or cholestenoic acid.

In some aspects, the cholesterol lowering drug normalizes cholesterol metabolism. In some aspects, an example of normalizing cholesterol metabolism can be a reduction in total cholesterol, serum cholesterol or pancreatic cholesterol. In some aspects, an example of normalizing cholesterol metabolism can be a reduction in cholesterol to levels present in subjects not having pancreatitis.

In some aspects, the cholesterol lowering drug can reduce pancreatitis-induced increases in total cholesterol and trypsin activity in the subject. In some aspects, the reduction in total cholesterol can be at least 10%, 15%, 20%, 25%, 30%, 35%, 40%, 45%, 50% or more reduction when compared to untreated subjects.

In some aspects, the cholesterol lowering drug reduces accumulation of LC3-II, p62, and/or ubiquitylated proteins in the subject.

In some aspects, the cholesterol lowering drug normalizes mitochondrial fragmentation or abrogates CER-induced increase in MLN64 in the subject.

### E. Compositions and Administration

As disclosed herein, are cholesterol lowering drugs and pharmaceutical compositions comprising the cholesterol lowering drugs and a pharmaceutical acceptable carrier. In some aspects, the cholesterol lowering drugs and pharmaceutical compositions comprising the cholesterol lowering drugs can be formulated for intravenous, subcutaneous, intradermal, intraperitoneal, intraocular, or intravitreal administration. The cholesterol lowering drugs and pharmaceutical compositions comprising the cholesterol lowering drugs can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The cholesterol lowering drugs and pharmaceutical compositions comprising the cholesterol lowering drugs as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the peptides disclosed herein. Thus, compositions can be prepared for parenteral administration that includes the peptides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The cholesterol lowering drugs and pharmaceutical compositions comprising the cholesterol lowering drugs can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The cholesterol lowering drugs and pharmaceutical compositions comprising the cholesterol lowering drugs in solid form can be packaged in multiple single dose units, each containing a fixed amount of the above-mentioned agent or agents, such as in a sealed package of tablets or capsules. The cholesterol lowering drugs and pharmaceutical compositions comprising the cholesterol lowering drugs in solid form can also be packaged in a container for a flexible quantity, such as in a squeezable tube designed for a topically applicable cream or ointment.

The cholesterol lowering drugs and pharmaceutical compositions comprising the cholesterol lowering drugs described herein can also be formulated so as to provide slow, prolonged, or controlled release. For example, a controlled-release preparation is a pharmaceutical composition capable of releasing the peptides or compositions disclosed herein at a desired or required rate to maintain constant activity for a desired or required period of time.

The total effective amount of any of the cholesterol lowering drugs or pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of any of the cholesterol lowering drugs disclosed herein present within the pharmaceutical compositions described herein and used in the methods as disclosed herein applied to subjects (e.g., humans) can be determined by one of ordinary skill in the art with consideration of individual differences in age, weight, and other general conditions.

In some aspects, the compositions can be a combination of a cholesterol lowering drug and a second pancreatic therapy as described herein.

In the methods described herein, administration or delivery of the compositions can be via a variety of mechanisms. For example, the therapeutic can be formulated as a pharmaceutical composition.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

Amounts of cholesterol lowering drug or composition effective for the methods disclosed herein can depend on the severity of the disease and the weight and general state and health of the subject. Suitable regimes for initial administration and booster administrations are typified by an initial administration followed by repeated doses at one or more hourly, daily, weekly, or monthly intervals by a subsequent administration. For therapeutic uses, the cholesterol lowering drugs and compositions can include a pharmaceutically acceptable excipient. Such compositions can be formulated without undue experimentation for administration to a mammal, including humans, as appropriate for the particular application. Additionally, proper dosages of the compositions can be determined without undue experimentation using standard dose-response protocols. For example, a subject can receive any of the peptides or compositions disclosed herein one or more times per week (e.g., 2, 3, 4, 5, 6, or 7 or more times per week).

### F. Kits

Disclosed are kits comprising one or more of the compositions or pharmaceutical compositions comprising a cholesterol lowering drug described herein. In some aspects, the kits can further comprise a second pancreatic therapeutic and described herein.

### EXAMPLES

### A. Example 1: Dysregulation of Mannose-6-Phosphate-Dependent Cholesterol Homeostasis in Acinar Cells Mediates Pancreatitis

### 1. Introduction

Pancreatitis is a common, potentially fatal disease of the exocrine pancreas. Its pathogenesis remains obscure, and no specific or effective treatment is available (1, 2). Because of the general lack of access to human tissue, most studies use animal or ex vivo (cellular) models to investigate pathogenic mechanisms of pancreatitis (3). These models reproduce pathologic responses of human disease and the spectrum of their severity. Although defects in other cell types' function (e.g., ductal; ref. 4) can initiate pancreatitis, damaged acinar cells play a central role in the disease development (1, 5). The primary function of these cells is to synthesize, store, and export digestive enzymes, which relies on coordinated actions of cytoplasmic organelles to maintain membrane trafficking along secretory and endolysosomal pathways (5).

Recent studies indicate that lysosomal/autophagy pathways are impaired in acinar cells in both experimental and human pancreatitis (5-7). Further, genetic alterations specifically targeting autophagy or lysosomal function can cause spontaneous pancreatitis in mice (8-11). However, the mechanisms whereby impaired lysosomal/autophagy pathways cause disordering of key acinar cell organelles, e.g., the ER and mitochondria, and classic pancreatitis responses, such as inappropriate, intra-acinar trypsinogen activation, are poorly understood (5, 7).

Delivery of newly synthesized hydrolases to lysosomes involves covalent addition of mannose 6-phosphate (M6P) to asparagine residues of lysosomal hydrolases in the cis-Golgi network. The modified hydrolases then bind to one of the two transmembrane M6P receptors on vesicles that are subsequently directed toward endosomes to deliver their cargo to lysosomes (12-15). N-acetylglucosamine-1-phosphotransferase is a key enzyme in the M6P-tag formation; its catalytic a/B subunits are encoded by the GNPTAB gene (15-17). Ablation of Gnptab in mice (18, 19) causes pathologic changes in exocrine glands, including the pancreas, the main manifestation of which is accumulation of large vacuoles identified by electron microscopy as autolysosomes (19). Accumulation of large vacuoles in acinar cells is a long-noted response of human and experimental pancreatitis (20, 21); it has been shown that it is mediated by defective autophagy caused by lysosomal dysfunction (6, 22). Both Gnptab−/− pancreas and pancreatitis models display impaired activities and processing of lysosomal hydrolases. These similarities prompted an investigation of how the blockage of a key endolysosomal pathway affects the exocrine pancreas.

Here, it is shown that Gnptab ablation greatly perturbs the endolysosomal system and autophagy in the exocrine pancreas; in particular, it inhibited endocytic recycling, blocked autophagic flux, and stimulated autophagosome formation. But the key unexpected finding is that the M6P pathway is critical for maintaining exocrine pancreas cholesterol homeostasis. Cholesterol levels and its distribution among the plasma membrane and organelles (endolysosomes, ER, and mitochondria) regulate their stability and functions (23-26). Disordering of cholesterol metabolism should be particularly detrimental for acinar cell homeostasis and functions, as zymogen granule maturation, secretion, and membrane retrieval and recycling after exocytosis depend on efficient turnover of membranes' lipid components. However, essentially nothing is known about acinar cell cholesterol metabolism in healthy pancreas and its changes in experimental pancreatitis and human disease.

Gnptab ablation caused profound changes in acinar cell cholesterol synthesis, uptake, and subcellular distribution, resulting in accumulation of nonesterified cholesterol in late endosomes/lysosomes (LE/Ly) and autophagic vacuoles and its reduction in the plasma membrane. Gnptab−/− mice spontaneously developed pancreatitis with all major characteristics of human disease. Similar dysregulation of cholesterol metabolism in experimental WT mouse and ex vivo pancreatitis models were found, while reducing acinar cell cholesterol overload alleviated disease. Importantly, manifestations of disordered cholesterol metabolism were observed in human pancreatitis. The results reveal the essential role of the M6P pathway in maintaining exocrine pancreas homeostasis and function, and implicate cholesterol disordering in the pathogenesis of pancreatitis.

### 2. Results

i. Blockage of the M6P Pathway Disrupts the Endolysosomal System, Autophagy, and Endocytosis in the Exocrine Pancreas

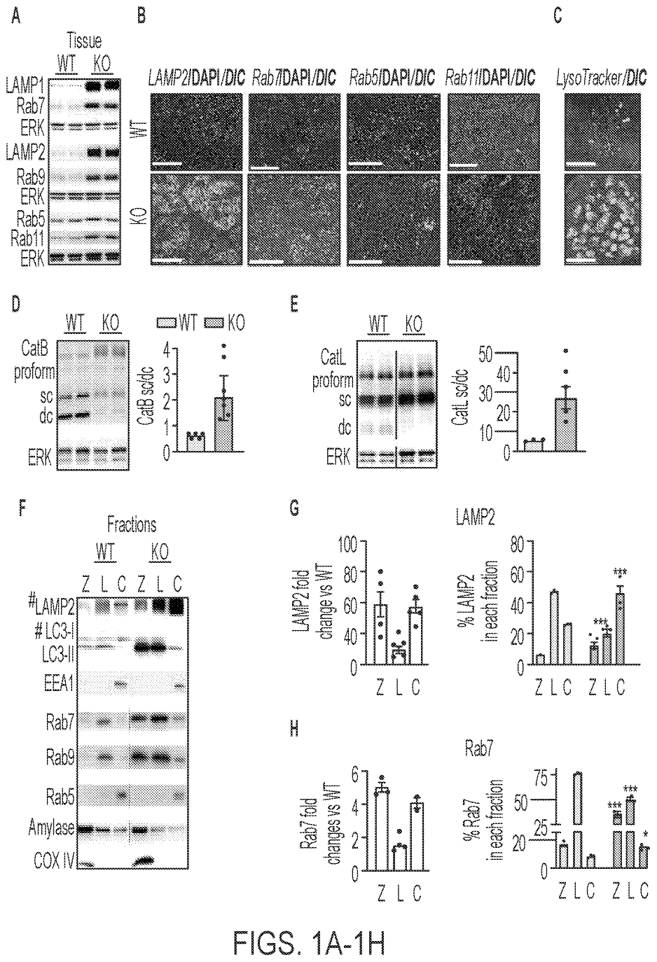

Gnptab ablation causes LE/Ly expansion and subcellular redistribution. Gnptab ablation greatly increased pancreatic levels of LE/Ly markers LAMPI, LAMP2, Rab7, and Rab9 (FIG. 1A). Their immunofluorescence (IF) pattern changed from small puncta in WT mice to large clumps of various shapes in Gnptab−/− (KO) pancreas (FIG. 1B). The expansion and clumping of acidic organelles was also seen with LysoTracker (FIG. 1C). In contrast, the levels of early (Rab5) and recycling (Rab11) endosomal markers increased only marginally (FIG. 1A), and their IF staining (FIG. 1B) remained dotted (although the size and cellular distribution of puncta changed somewhat).

Lysosomal hydrolases, such as cathepsins B and L (CatB and CatL), are synthesized as pro-proteins and undergo proteolytic processing first in endosomes, generating a partially processed single-chain (sc) form, and then in lysosomes where at acidic pH they mature into the fully processed, active double-chain (dc) form (27). Gnptab deficiency compromised cathepsins' maturation, as evidenced by increased amounts of the pro-form and 3- to 4-fold increases in sc/dc ratios for both CatB and CatL (FIGS. 1, D and E). Reduced sc-to-dc conversion in KO pancreas was also observed for CatD (19). The inhibition of cathepsin processing/maturation indicates that the delivery of these proteases to LE/Ly in pancreas, indeed, depends on the M6P pathway, the involvement of which is organ and cell specific (19, 28).

Of note, the pronounced reduction in the levels of the fully mature de form (FIGS. 1, D and E) did not abrogate CatB and CatL activities (FIGS. 1, J and K), indicating that the partially processed sc form possesses considerable catalytic activity.

To further analyze the effect of Gnptab ablation on the endolysosomal system, (FIG. 1, F-N) commonly applied pancreatic tissue fractionation were used (6, 29-31) to generate 3 subcellular fractions: Z (1300 g pellet), L (12,000 g pellet), and C (12,000 g supernatant). In WT pancreas the heavier fraction Z was enriched in zymogen granule markers (i.e., amylase) and contained mitochondria (COX IV), whereas the cytosol-containing fraction C was selective for the markers of early endosomes (EE), Rab5 and EEA1 (FIG. 1F; see also ref. 6). Fraction L in WT was enriched in LE/Ly (LAMP2, Rab7, and Rab9) and exhibited the highest CatB and CatL activities (FIG. 1, F-K). This subcellular distribution is characteristic of healthy cells in which the level of lysosomal proteolytic activity correlates with the amount of LE/Ly.

Gnptab ablation altered the subcellular distribution of LE/Ly and cathepsin activities in the pancreas. LAMP2, Rab7, and Rab9 increased in all 3 fractions, but fraction L exhibited the smallest increase, resulting in LE/Ly shift away from fraction L to Z and C (FIGS. 1, G and H). In contrast, the distribution of EE (Rab5 and EEA1), zymogen granules (amylase), and mitochondria (COX IV) did not significantly change (FIGS. 1, F and I). CatB and CatL activities increased in fraction C but decreased by half in fractions Z and L, altering cathepsins' subcellular distribution profile (FIGS. 1, J and K). Thus, the substantial LE/Ly expansion in KO pancreas was not matched/counterbalanced by corresponding increases in cathepsin activity (as would be expected in cells with competent LE/Ly), which was evidenced by a greater than 100-fold decrease in the ratio of CatB activity to LAMP2 level across the fractions (FIG. 1M).

The data on subcellular redistribution indicate structural changes in the exocrine pancreas endolysosomal system. Gnptab ablation caused an imbalance between LE/Ly expansion and their reduced proteolytic capacity; one underlying mechanism could be impaired processing/maturation of cathepsins.

ii. Gnptab Ablation Stimulates Autophagosome Formation and Blocks Autophagic Flux

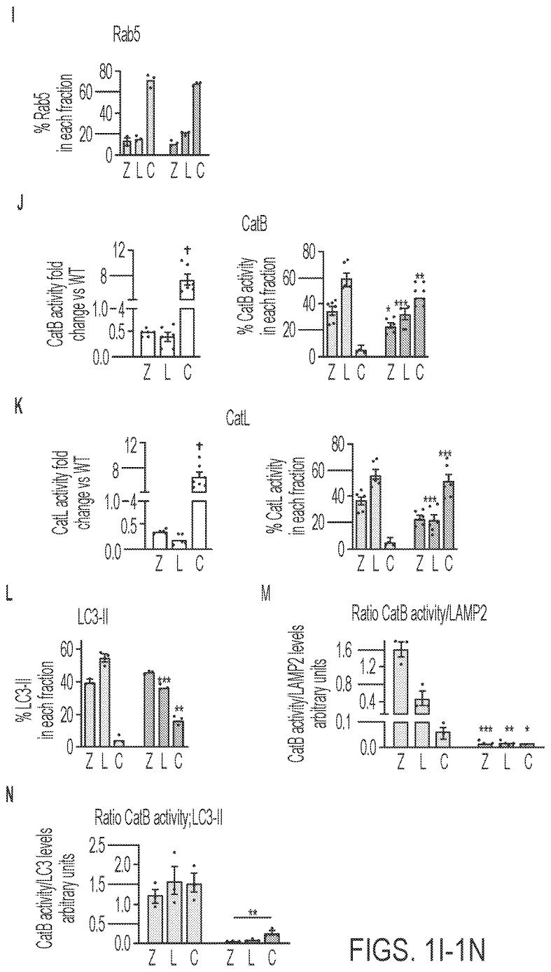

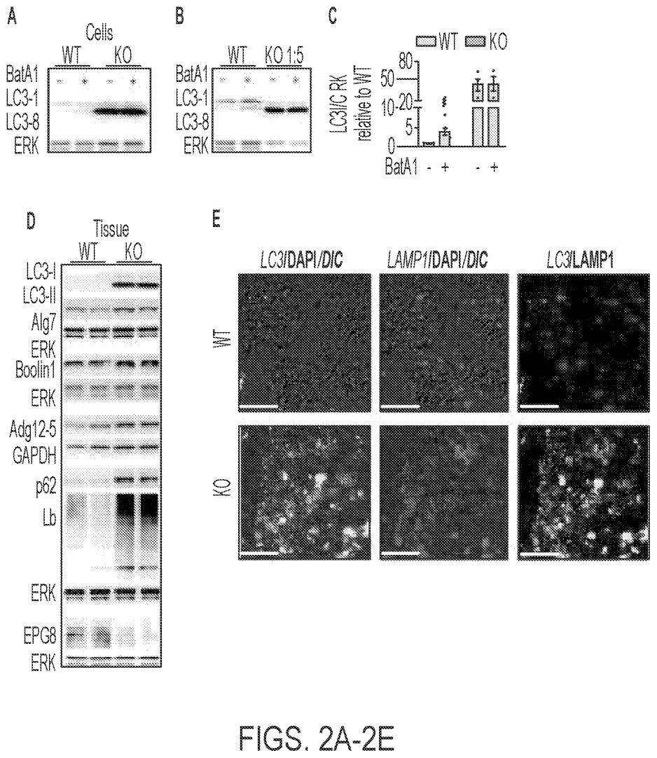

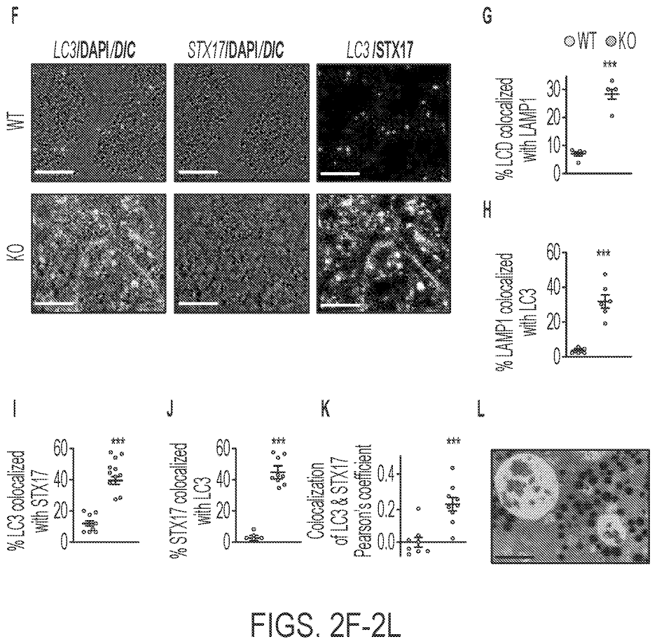

Gnptab ablation greatly increased pancreatic levels of LC3-II (FIG. 1F and FIG. 2, A-D), a marker of autophagic vacuoles (also observed in ref. 19). LC3-II levels in KO pancreas increased in all 3 fractions, but more significantly in C, resulting in subcellular redistribution of LC3-II (FIGS. 1, F and L). The CatB activity/LC3-II ratio markedly decreased (FIG. 1N), especially in fractions Z and L, similar to that for LAMP2 (FIG. 1M).

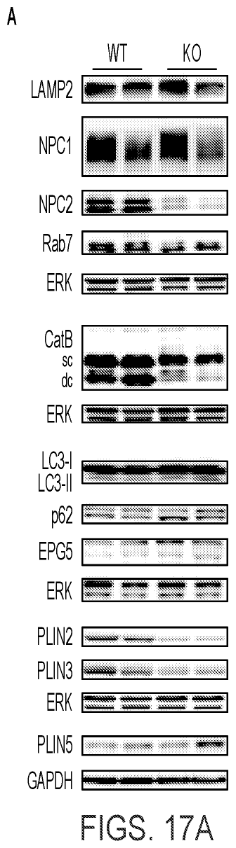

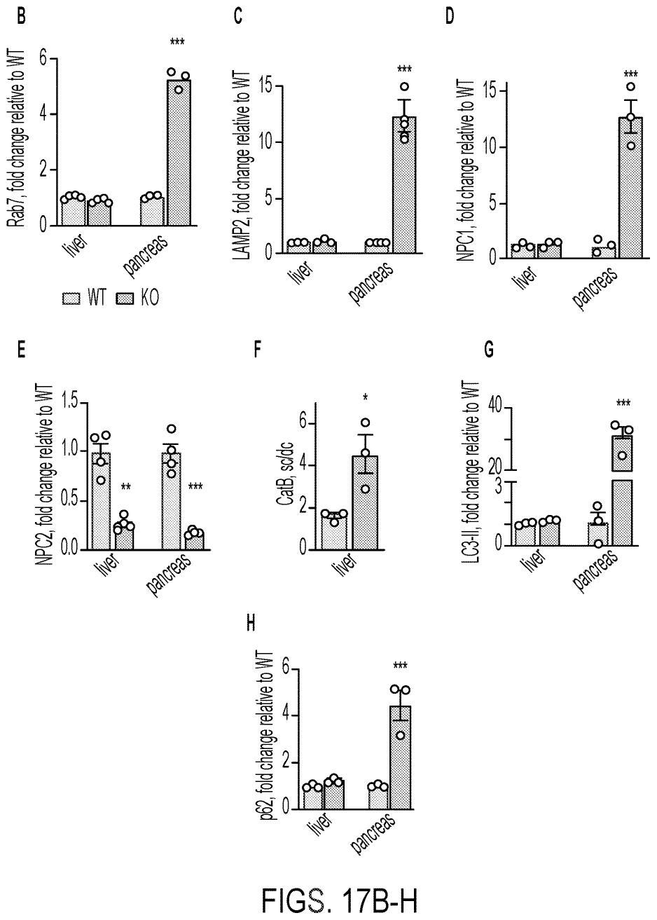

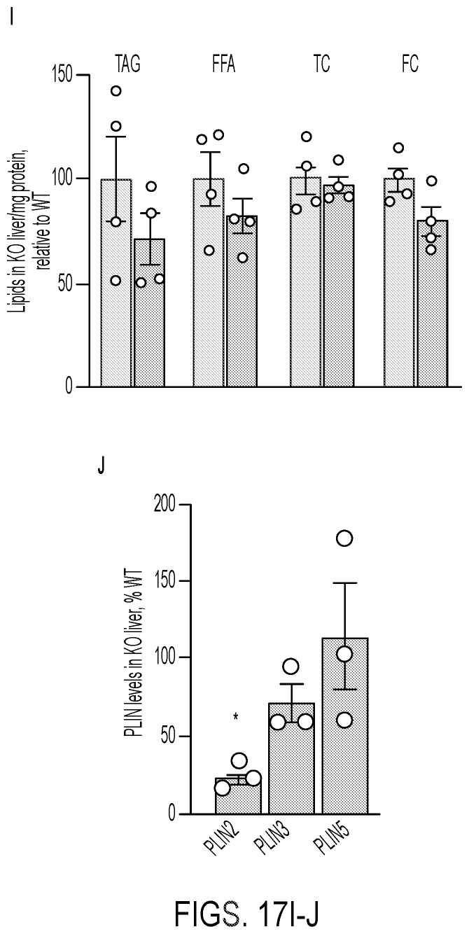

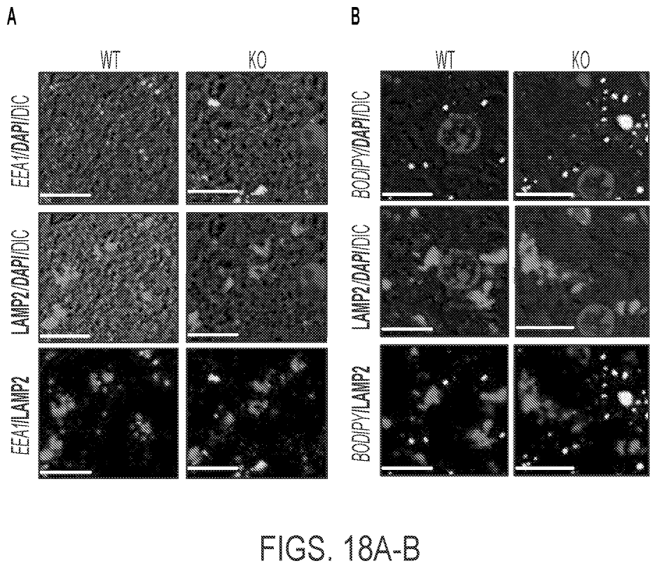

To further assess the effects of Gnptab KO on autophagy, autophagic flux analysis was performed by measuring changes in LC3-II in acinar cells incubated with and without the lysosomal v-ATPase inhibitor, bafilomycin A1 (BafA1) (FIG. 2, A-C). The number of autophagic vacuoles in a cell is a net balance between autophagosome formation and their degradation through autophagic flux. Thus, the increase in LC3-II observed in KO pancreas (FIG. 1F and FIG. 2D; see also FIG. 17G) could have resulted from both induction of autophagosome formation and the inhibition of flux. BafA1 blocks lysosomal/autophagic degradation but does not affect autophagosome formation; thus, in the presence of BafA1 any difference in cellular LC3-II levels between 2 conditions (e.g., KO vs. WT) is due solely to changes in autophagosome formation (31, 32). In the presence of BafA1, LC3-II levels were approximately 11-fold higher in KO compared with WT (FIG. 2, A-C), indicating strong induction of autophagosome formation in Gnptab−/− acinar cells.

On the other hand, the magnitude of LC3-II increase elicited by BafA1 in a given condition (e.g., WT cells) reflects the efficiency of autophagic degradation in this condition; the greater the LC3-II increase, the greater was the contribution of autophagic flux abrogated by BafA1 (31, 32). In WT acinar cells, BafA1 increased LC3-II approximately 4-fold (FIG. 2C), reflecting efficient basal autophagic flux, the blockage of which by BafA1 caused accumulation of LC3-positive autophagic vacuoles (31). Remarkably, in KO cells BafA1 elicited no increase in LC3-II (FIG. 2, A-C), indicating a complete block of autophagic degradation and demonstrating a critical role of the M6P pathway in maintaining efficient pancreatic autophagy. Together, the block of autophagic flux and rise in autophagosome formation increased the basal LC3-II level in Gnptab−/− cells approximately 37-fold compared with WT (FIG. 2C).

In accord with these results, marked increases in pancreatic levels were observed of not only LC3-II but also the autophagy substrate p62/SQSTM1 and ubiquitylated proteins (FIG. 2D), corroborating impaired autophagic degradation (32). Other mediators of autophagosome formation, Beclin1/ATG6, ATG5 (i.e., ATG12-ATG5 conjugate), and ATG7, were moderately elevated (FIG. 2D). Gnptab ablation did not block autophagosome fusion with lysosomes, as evidenced by dramatic increases in LC3-II colocalization with LAMP1 and acquisition of STX17, a key mediator of this fusion, into LC3-positive puncta in KO acinar cells (FIG. 2, E-K). Also, numerous enlarged autolysosomes containing partially degraded material were seen on electron micrographs of KO pancreas (FIG. 2L and ref. 19).

Aa marked decrease in KO pancreas of EPG5 was found (FIG. 2D), a protein that promotes autolysosomal proteolytic activity and genetic deletion of which causes formation of nondegradative autolysosomes (33). Together with LC3-II subcellular redistribution and the markedly decreased CatB activity/LC3-II ratio (FIGS. 1, L and N), these data support the notion of an imbalance between increased autophagosome formation and low proteolytic activity in LC3-positive autophagic vacuoles.

Thus, Gnptab ablation stimulates acinar cell autophagy but blocks its completion (autophagic flux), resulting in accumulation of autolysosomes with low degradative capacity.

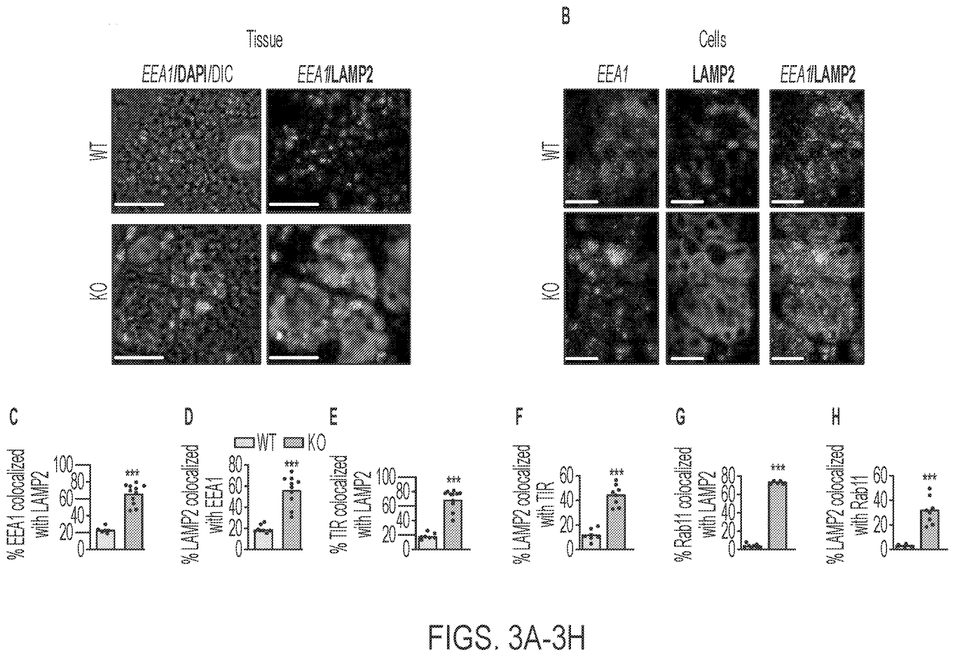

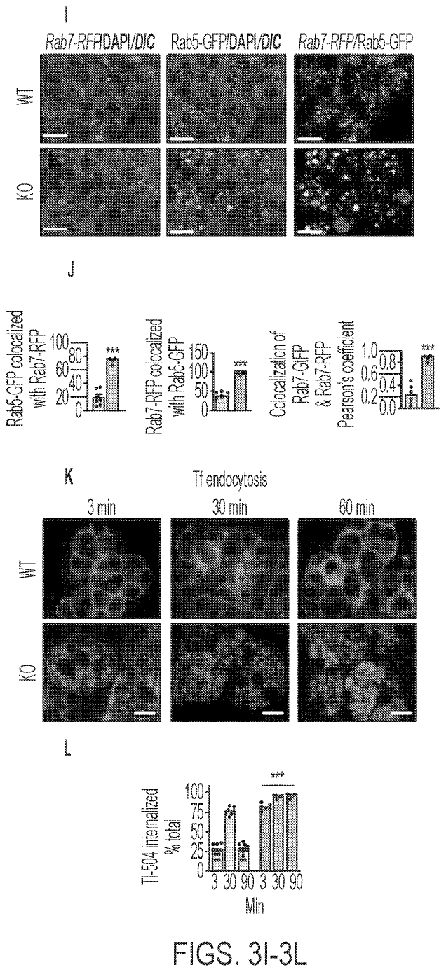

Gnptab KO impairs EE-to-LE maturation and endocytic recycling. Transport of endocytosed cargos to the lysosome depends on EE maturation into LE (34, 35), which involves transient formation of hybrid compartments containing markers of both EE (e.g., Rab5) and LE (Rab7, LAMP2). In WT pancreas, only approximately 20% of the EE markers EEA1 and transferrin receptor (TfR), and approximately 4% of the recycling endosomal marker Rab11 colocalized with LAMP2; these values increased up to approximately 70% in KO pancreas (FIG. 3, A-H, and FIG. 12), indicating impaired EE maturation. IF images (FIGS. 3, A and B, and FIG. 12) showed EE markers trapped within large organelles decorated with ring-like LAMP2, most clearly seen in KO acinar cell preparations (FIG. 3B). Further, Gnptab deficiency increased Rab5/Rab7 colocalization several-fold in acinar cells double-transduced with Rab5a-GFP and Rab7a-RFP (FIGS. 3, I and J). Thus, EE maturation is impaired in the Gnptab−/− pancreas, causing accumulation of hybrid endosomes that bear markers of both EE and LE.

The iron-carrying protein transferrin (Tf) is endocytosed upon binding its receptor (TfR), releases iron, and is recycled back to the plasma membrane; this process serves as an archetypical model of receptor-mediated cargo delivery (35). Endocytosis of fluorescently labeled Tf was measured (FIGS. 3, K and L), the uptake of which was initiated by transferring acinar cells from 0° C. to 37° C. Immediately (3 minutes) after initiating endocytosis in WT cells, the labeled Tf was mostly (~75%) present on the plasma membrane; after 30 minutes, the Tf-TfR complex accumulated in EE, and by 90 minutes it largely returned to the cell surface. By contrast, in Gnptab−/− cells Tf internalization was already prominent at 3 minutes (indicating enhanced Tf endocytosis) but there was no recycling back to the plasma membrane at 90 minutes (FIGS. 3, K and L). The results indicate inhibition of endocytic recycling in KO acinar cells; one reason for this could be impaired EE-to-LE maturation.

iii. The M6P Pathway is Critical for Maintaining Pancreatic Cholesterol Homeostasis

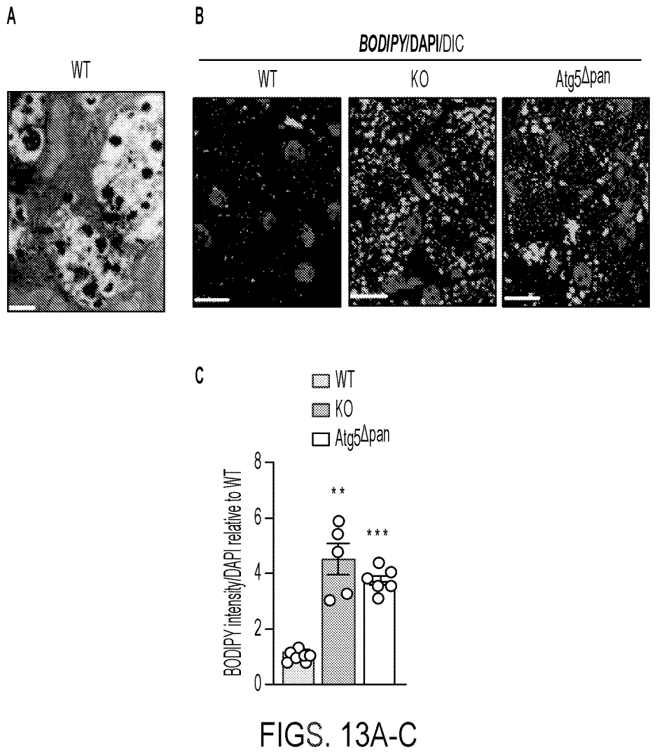

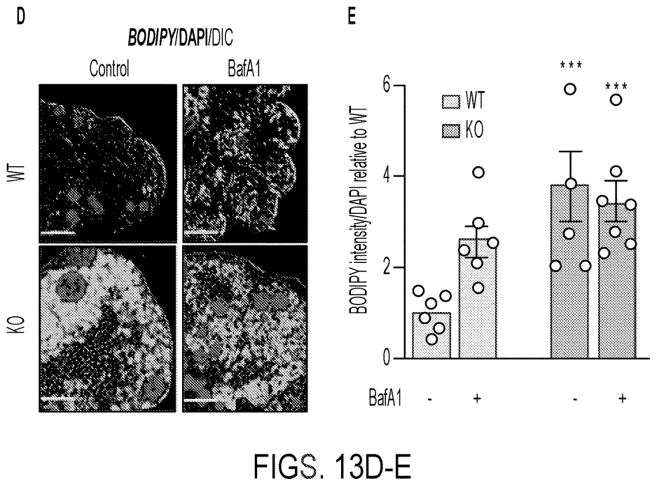

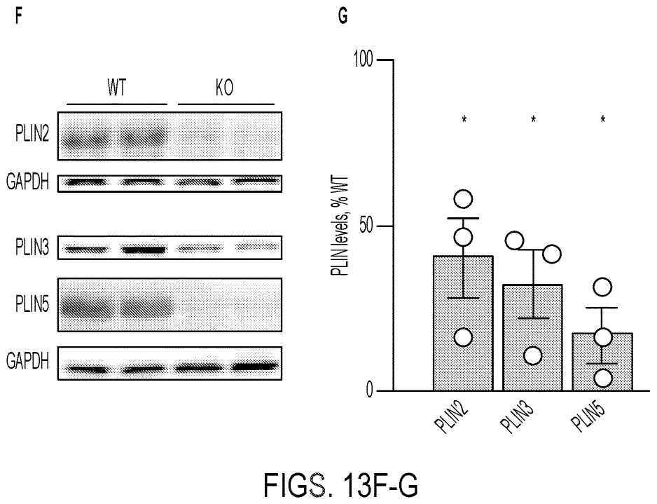

Gnptab KO perturbs pancreatic lipids. Numerous lipid-like inclusions were seen on electron micrographs of KO pancreas, indicating that the M6P pathway modulates acinar cell lipid metabolism (FIG. 13A). Whereas WT acinar cells contained few lipid droplets, blockage of autophagy by Gnptab ablation or in mice with pancreas-specific knockout of ATG5 (Atg5Δpan), a key mediator of autophagosome formation, increased neutral lipids in the pancreas approximately 4-fold (FIGS. 13B and C), as measured with the neutral lipid stain BODIPY 493/503 (36). Similarly, BafA1 treatment of WT acinar cells increased BODIPY staining approximately 3-fold, and there was no additional effect of BafA1 on the greatly elevated BODIPY staining in Gnptab−/− cells (FIGS. 13D and E). (Note that the pattern of BODIPY staining was different in BafA1-treated WT vs. KO cells.) These results reveal a key role of autophagy (lipophagy; ref. 37) in maintaining neutral lipid metabolism in the exocrine pancreas.

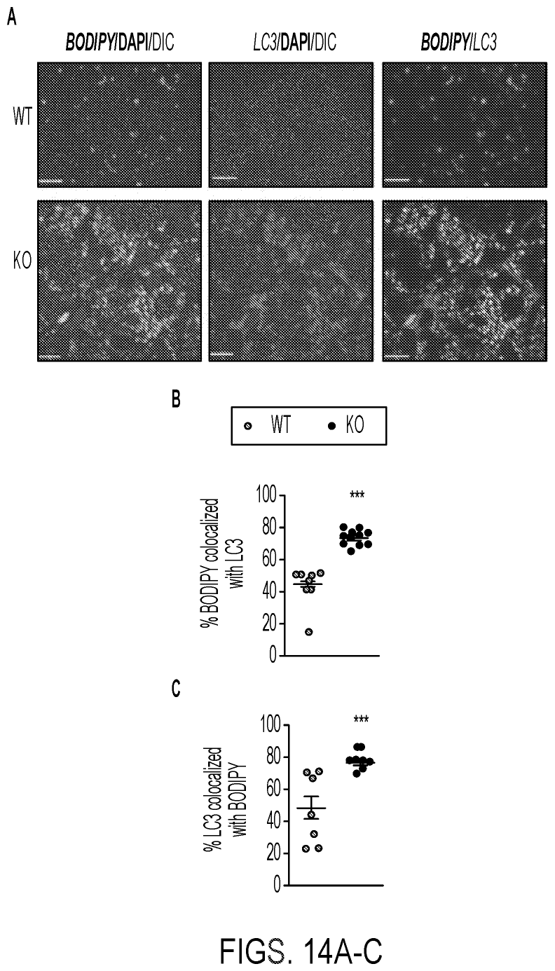

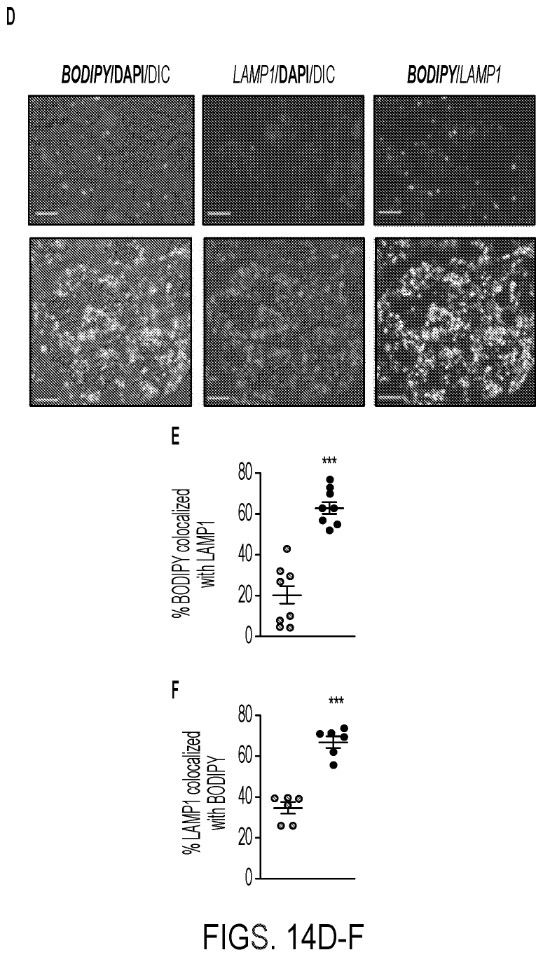

Accumulation of neutral lipids in KO pancreas was associated with a decrease in perilipins (FIGS. 13F and G), proteins essential for the integrity of lipid droplet membranes (38). Increased BODIPY colocalization was also found with both LC3 and LAMP1 (FIG. 14). Thus, Gnptab ablation caused accumulation of neutral lipids in LE/Ly and autophagic vacuoles, while decreasing their cytosolic storage.

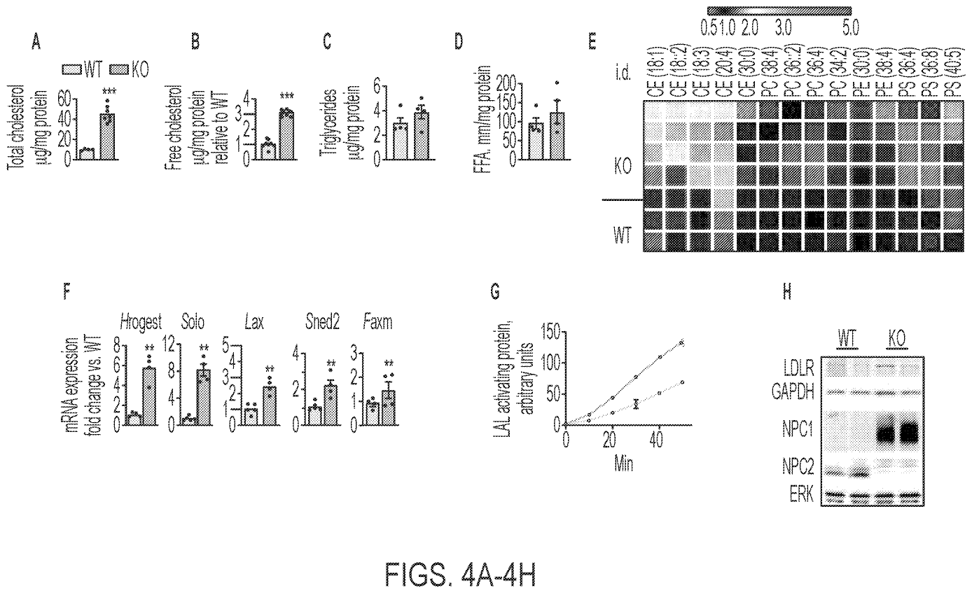

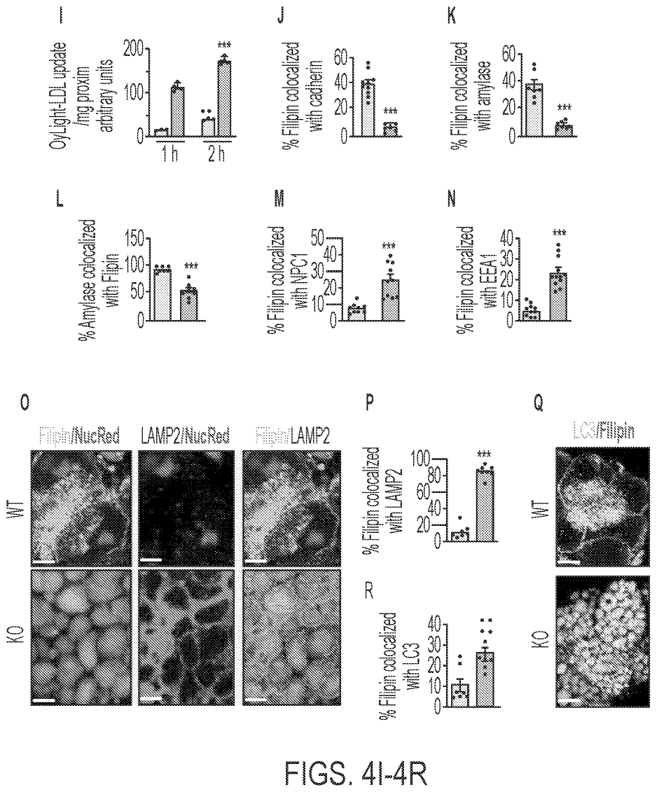

Gnptab KO disrupts cholesterol metabolism in the exocrine pancreas. Gnptab ablation increased both the total and nonesterified (free) cholesterol content in pancreas 3- to 4-fold (FIGS. 4, A and B), while it did not significantly elevate triglyceride and free fatty acid levels (FIGS. 4, C and D). Corroborating these data, LC-MS/GC-MS analysis showed several-fold increases in major cholesterol esters in KO pancreas, but not in other classes of lipids tested (FIG. 4E). Activation of several mechanisms that increase free cholesterol were found: the expression of genes mediating cholesterol synthesis (FIG. 4F), activity of lysosomal acid lipase (LAL) (FIG. 4G), the level of low-density lipoprotein receptor (LDLR) (FIG. 4H), and uptake of exogenous LDL (FIG. 4I).

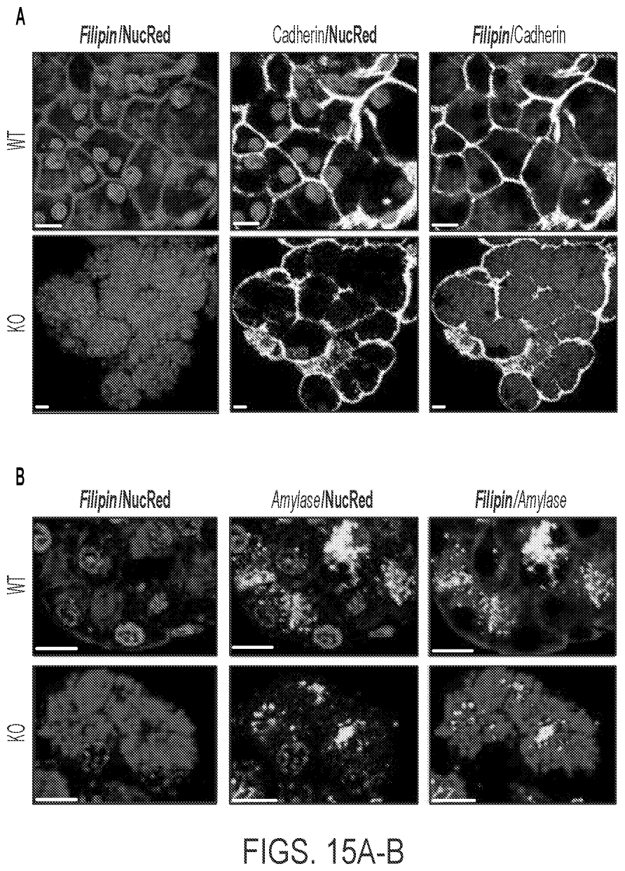

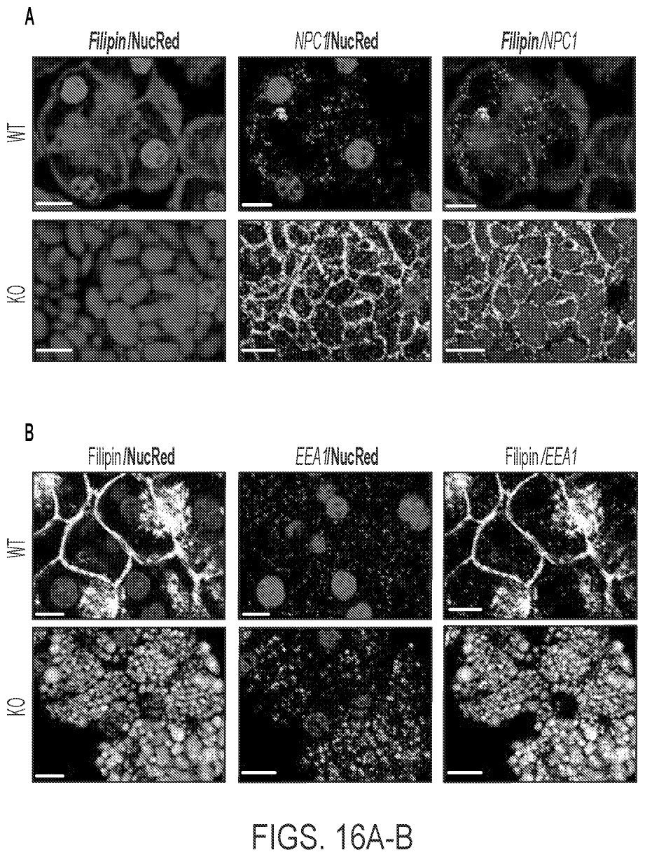

In WT acinar cells (FIG. 4, J-L, and FIG. 15) approximately 40% of filipin, a fluorescent probe for free cholesterol, localized to the plasma membrane (based on its marker cadherin), and another approximately 40% to zymogen granules (based on amylase), a distribution pattern similar to that of other secretory cells (23). Only 5% to 10% of the filipin probe colocalized with markers of LE/Ly (LAMP2, NPC1), EE (EEA1), or autophagic vacuoles (LC3; FIG. 4, M-R, and FIG. 16). By contrast, in Gnptab−/− acinar cells approximately 84% of filipin accumulated in organelles wrapped with LAMP2-positive membranes (FIGS. 4, O and P); a similar colocalization pattern was seen for filipin and NPC1 on IF images (FIG. 16A). Free cholesterol colocalization with EEAl and LC3 also increased several-fold in KO cells, whereas it markedly decreased in both the plasma membrane and zymogen granules (FIGS. 4, J-N, Q, and R, and FIGS. 15 and 16).

NPC1 and NPC2 proteins mediate transport of endocytosed cholesterol through LE/Ly (39). Similar to other LE/Ly membrane proteins (LAMP2, Rab7), NPC1 levels greatly increased in KO pancreas (FIG. 4H and FIG. 17). By contrast, it was observed that a marked decrease in NPC2 level in KO pancreas (FIG. 4H and FIG. 17)—in accord with the findings that, unlike NPC1, NPC2 targeting to LE/Ly is strictly dependent on the M6P pathway (28, 40).

Thus, blockage of the M6P pathway causes profound disruption of cholesterol homeostasis in the exocrine pancreas. This was manifested by a marked decrease in plasma membrane cholesterol, critical for maintaining conformation of ion channels and receptors and cell viability (23-26); reduction of cholesterol in membranes of zymogen granules (where it is necessary for regulated secretion; ref. 41); and cholesterol accumulation in LE/Ly and autolysosomes, reducing its availability for the cell's needs. Likely as a compensatory response, Gnptab−/− acinar cells upregulated both cholesterol synthesis and uptake. Notably, no significant changes were observed in serum cholesterol levels in Gnptab−/− mice.

Gnptab KO effects on the endolysosomal system and cholesterol are organ specific. Gnptab−/− livers exhibited impaired CatB processing (manifested by a decrease in its dc form) and reduced NPC2 levels (FIGS. 17A, E, and F), as seen in KO pancreas. However, there was no upregulation of LAMP2, Rab7, NPC1, LC3-II, and p62, and no decrease in EPG5 in KO liver (FIGS. 17A-D, G, and H). Gnptab deficiency did not change liver triglycerides, free fatty acids, or total and free cholesterol (FIG. 171). Perilipin 2 (PLIN2) levels decreased in KO liver, but there was little or no reduction in PLIN3 and PLIN5 (FIGS. 17A and J) as seen in the pancreas (FIGS. 13F and G). Gnptab ablation did not increase EEA1 colocalization with LAMP2 in the liver (FIG. 18A) or BODIPY colocalization with LAMP2, indicative of neutral lipid accumulation in LE/Ly (FIG. 18B).

Thus, overall, blockage of the M6P pathway did not significantly perturb the endolysosomal system and neutral lipids in the liver. In contrast to the pancreas, Gnptab deficiency in the liver did not cause LE/Ly expansion, accumulation of hybrid endosomes, or impaired autophagy.

iv. Gnptab−/− Mice Develop Pancreatitis

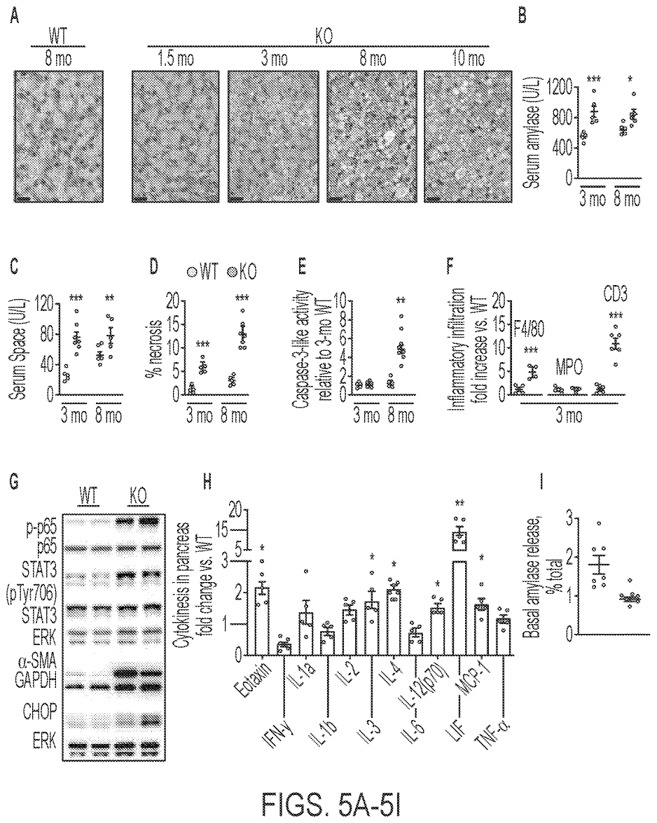

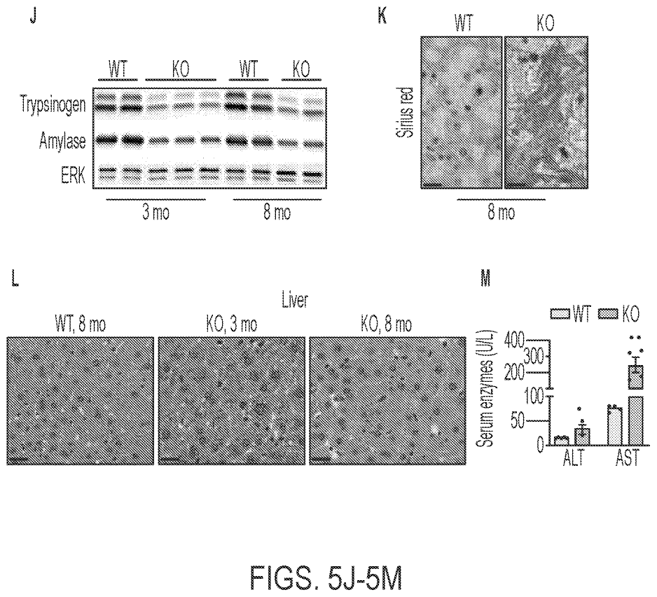

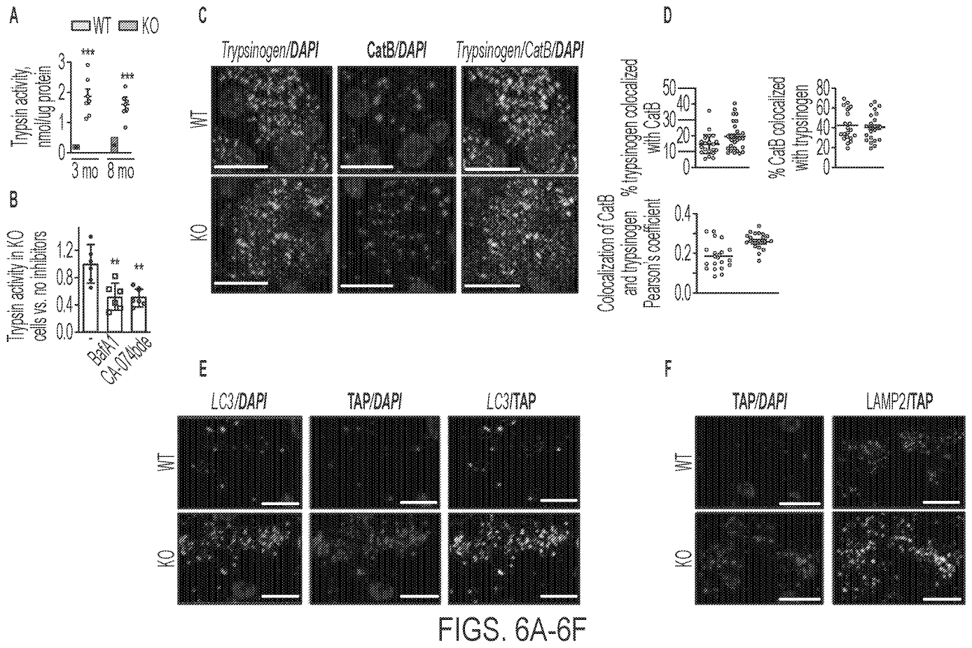

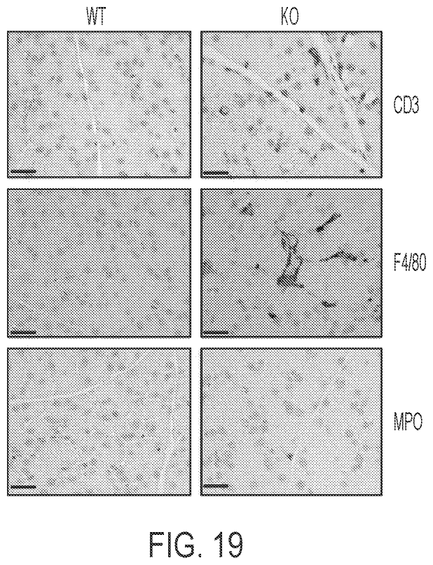

Acinar cell vacuolization, observed with light microscopy, was evident in 1.5-month-old KO mice, and vacuole size and pancreas disorganization increased with age (FIG. 5A). In addition to histopathologic changes, by 3 months Gnptab−/− mice developed signature responses of pancreatitis (1, 3): increased levels of serum amylase and lipase, inflammation, parenchymal necrosis, and increased caspase-3-like activity indicative of apoptosis (FIG. 5, B-F); as well as an upsurge in intrapancreatic trypsin activity (see below). Inflammatory infiltration in the pancreas was driven by macrophages and T cells, and not neutrophils (FIG. 5F and FIG. 19), which is a pattern characteristic of a chronic inflammatory response. Activation (i.e., phosphorylation) of the proinflammatory transcription factors NF-κB and STAT3 in KO pancreas was observed (FIG. 5G) and increased levels of cytokines and chemokines, such as LIF, IL-3, IL-4, and CCL11/eotaxin-1 were observed (FIG. 5H). Pancreatic levels of CHOP, an ER stress marker and cell death mediator, markedly increased (FIG. 5G); those of amylase and trypsinogen decreased (FIG. 5J), indicating exocrine pancreas atrophy. Basal (unstimulated) amylase secretion was inhibited in KO acinar cells (FIG. 5I). Pancreatic fibrosis was evidenced by upregulation of a-SMA, a marker of activated stellate cells (FIG. 5G), and by Sirius red staining (FIG. 5K). In contrast, Gnptab−/− liver displayed no vacuolization or other histopathologic changes, and the serum markers of liver damage, alanine (ALT) and aspartate (AST) aminotransferases, were only mildly elevated (FIGS. 5, L and M).

The results show that blockage of the M6P pathway causes spontaneous pancreatitis in mice.

v. Trypsin Activity Cholesterol-Dependently Increases in Gnptab−/− Pancreas

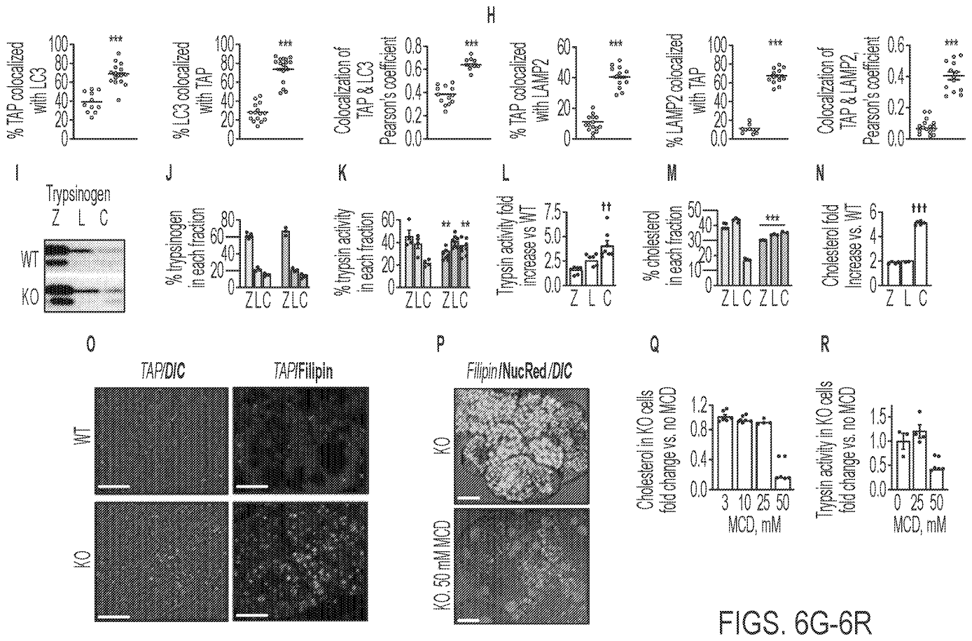

Gnptab deficiency greatly increased intrapancreatic trypsin activity (FIG. 6A), also evidenced by increased levels of trypsinogen activation peptide (TAP) (FIGS. 6, E and F), an oligopeptide cleaved off trypsinogen during its conversion to active trypsin (6, 42). The inappropriate, intra-acinar trypsinogen activation is a hallmark of pancreatitis but its mechanism, and the site(s) where it occurs, remain obscure (5, 43). In experimental pancreatitis it is mediated by CatB (6, 43, 44), and, indeed, the CatB inhibitor CA-074Me reduced trypsin activity in Gnptab−/− acinar cells by half (FIG. 6B). In normal secretory cells, the nascent immature secretory granules, due to imperfect sorting, have an admixture of lysosomal enzyme precursors, although the majority of their content are secretory proteins. During granule maturation, hydrolases are removed through an M6P receptor-mediated mechanism (discussed in ref. 5). Because in KO pancreas cathepsins lack the M6P tag, this mechanism is compromised and one might presume that CatB is retained (i.e., mis-sorted) in mature zymogen granules (19) and activates trypsinogen there. However, CatB colocalization with trypsinogen did not increase in KO pancreas (FIGS. 6, C and D), arguing against the predominance of this mechanism. (Note that CatB colocalization with trypsinogen is fairly robust in WT acinar cells, Pearson's coefficient; FIG. 6D).

Instead, Gnptab ablation greatly increased TAP colocalization with LAMP2 and LC3 (FIG. 6, E-H). Further, BafA1 reduced trypsin activity in KO acinar cells by half (FIG. 6B). These results indicate trypsin localization in acidic CatB-containing organelles, as also supported by the subcellular fractionation data. In WT pancreas, trypsinogen mostly localized to fraction Z (enriched in zymogen granules) and the bulk of trypsin activity was in the Z and L fractions (FIG. 6, I-K). Gnptab deficiency did not change the subcellular distribution of trypsinogen (FIGS. 6, I and J; as also seen for amylase, another zymogen granule protein, FIG. 1F). However, the percentage of trypsin activity in fraction Z decreased in KO pancreas, while its largest (~4.7-fold) increase was in fraction C (FIGS. 6, K and L), which contained EEA1 and Rab5 and harbored large increases in LC3-II and LAMP2 levels and CatB activity (FIGS. 1, F, G, I, J, and L).

The subcellular distribution of trypsin activity in KO pancreas was similar to that of cholesterol, in that the greatest increase in both was in fraction C (FIG. 6, K-N). TAP colocalized with filipin-positive structures (FIG. 60); furthermore, reducing cholesterol content with its chelator, methyl-β-cyclodextrin (MCD), greatly decreased trypsin activity in KO cells (FIG. 6, P-R). Of note, Gnptab−/− acinar cells were highly resistant to MCD, with the cholesterol-reducing effect evident only at 50 mM MCD (FIGS. 6, Q and R) instead of the usual 1 to 5 mM (45).

The results show that the M6P pathway regulates intrapancreatic trypsin activity in a cholesterol-dependent manner, and implicate endosomal and autophagic compartments—but not zymogen granules—as the sites of trypsinogen activation in Gnptab−/− acinar cells. The data are consistent with the notion that the colocalization organelles may originate from fusion of trypsinogen-containing autophagosomes with endosomes (such as the hybrid EE/LE) in which the partially processed (sc form) CatB is active; these amphisomes can subsequently fuse with lysosomes. The observed increases in both trypsin activity and cholesterol in fraction C could be explained by accumulation of low-density, cholesterol-rich autophagic vacuoles in this fraction. The mechanisms through which cholesterol regulates trypsinogen activation in pancreatitis remain to be determined.

vi. Gnptab Ablation Causes Mitochondrial Dysfunction in the Pancreas

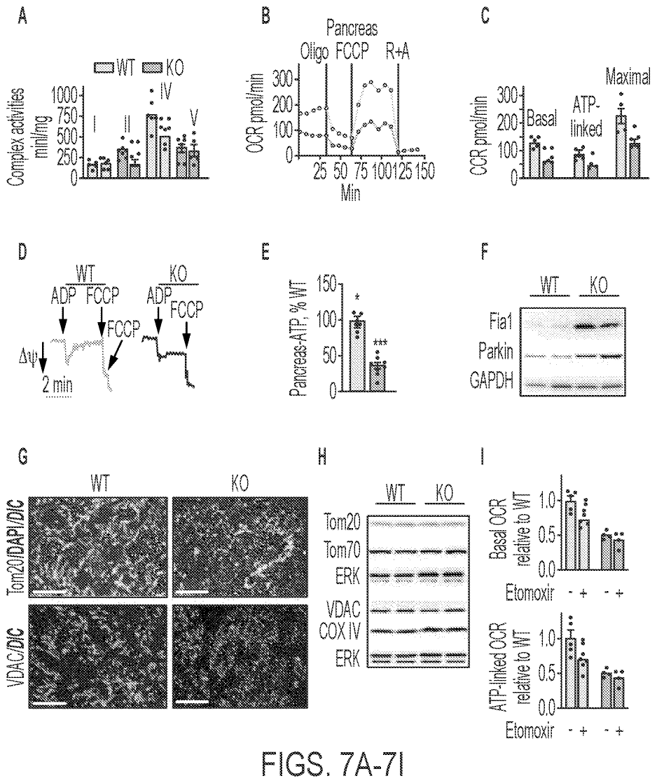

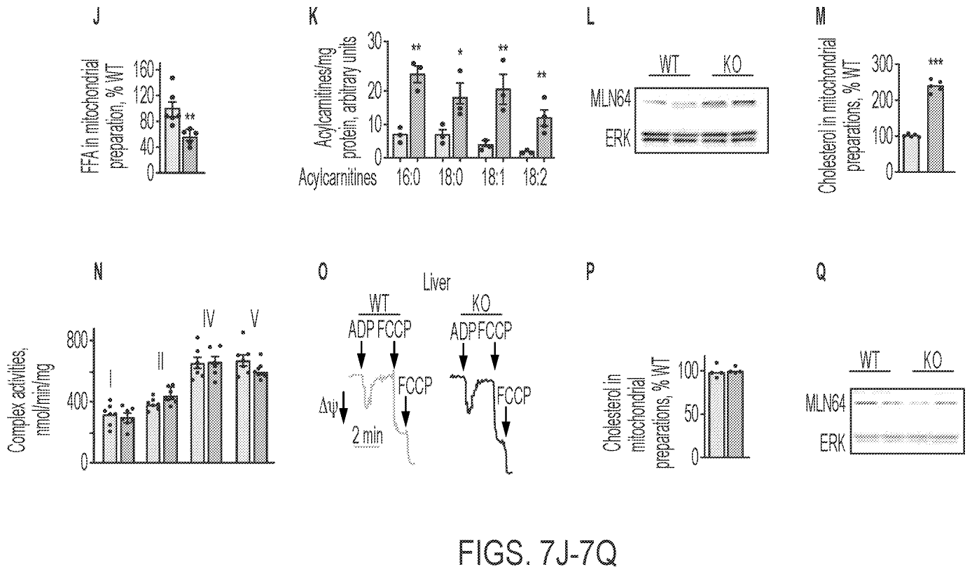

Mitochondrial dysfunction drives pancreatitis in experimental mouse and cellular models and is associated with human disease (5, 46-48). However, the role of the endolysosomal system, in particular the M6P pathway, in pancreatic mitochondrial homeostasis has not been explored. Pronounced mitochondrial dysfunction in Gnptab−/− pancreas was found, manifested by reduced enzymatic activities of electron transport chain (ETC) complexes II and IV (FIG. 7A); decreased rates of basal, ATP-linked, and maximal oxygen respiration (oxygen consumption rate, OCR) were found (FIGS. 7, B and C); and a marked decrease in pancreatic ATP level were found (FIG. 7E). In WT pancreatic mitochondria, ADP-stimulated ATP synthesis caused a decrease in mitochondrial membrane potential (ΔΨm), which returned to basal levels after ADP was converted to ATP (FIG. 7D). In contrast, the ΔΨm recovery following ADP addition was greatly inhibited in Gnptab−/− mitochondria (FIG. 7D), indicating impaired ATP synthase function (46). Levels of Fis1, a protein involved in mitochondrial fragmentation, and Parkin, a mitophagy mediator (49), increased in KO pancreas (FIG. 7F). The increase in mitochondrial fragmentation was evidenced by IF of the mitochondria-resident proteins Tom20 and VDAC, which showed a disjoined and mostly circular pattern in KO pancreas, in contrast with the interconnected tubular mitochondrial network in WT (FIG. 7G). Of note, Gnptab ablation did not decrease immunoblot (IB) levels of mitochondrial markers, i.e., the outer mitochondrial membrane protein Tom70 and the inner mitochondrial membrane protein COX IV (FIG. 7H), indicating no decrease in the amount of mitochondria in KO pancreas.

Collectively, the results show a major role of the M6P pathway in maintaining acinar cell mitochondrial functions.

Some of the mitochondrial defects in Gnptab−/− pancreas could be the result of perturbed lipid metabolism. Specifically, ATP production in WT acinar cells is mediated by free fatty acid oxidation, as evidenced by inhibition of both basal and ATP-linked OCR with etomoxir (FIG. 7I), a β-oxidation inhibitor (50). In KO acinar cells, the respiration rates were much lower and not significantly reduced by etomoxir (FIG. 7I). One reason for this could be the approximately 2-fold decrease in free fatty acid content in KO pancreatic mitochondria (FIG. 7J), likely a consequence of reduced availability of lipid droplets (FIGS. 13 and 14) that deliver free fatty acids to mitochondria (51). With LC-MS/GC-MS, increased levels of long-chain acylcarnitines in KO pancreas were shown (FIG. 7K), an indication of impaired β-oxidation (52, 53).

It was further found that the cholesterol content of Gnptab−/− pancreatic mitochondria increased approximately 2.5-fold (FIG. 7M), which is damaging for mitochondrial functions (24, 54). Elevated cholesterol interferes with assembly of ETC complexes and thus could be responsible for their reduced activity and dysregulation of mitochondrial ATP synthesis (FIG. 7, A-E). One likely mechanism mediating mitochondrial cholesterol overload in KO pancreas is the increase in MLN64/STARD3 protein (FIG. 7L), which belongs to the STARD family of lipid transfer proteins that transport cholesterol directly from LE/Ly to mitochondria. This pathway is activated in conditions of inhibited cholesterol egress from lysosomes, and STARD protein levels increase in response to a defective supply of cholesterol to mitochondria via normal routes (24, 55, 56). A similar paradoxical effect of mitochondrial cholesterol overload despite disrupted cholesterol turnover, as well as increased levels of STARD protein, was observed in Niemann-Pick type C (NPC) disease models (55).

Notably, in contrast to pancreas, Gnptab deficiency in liver mitochondria did not alter ETC complex activities, the ADP-mediated ΔΨm response, cholesterol content, or MLN64 levels (FIG. 7, N-Q).

vii. Cholesterol Metabolism is Disrupted in Experimental Pancreatitis

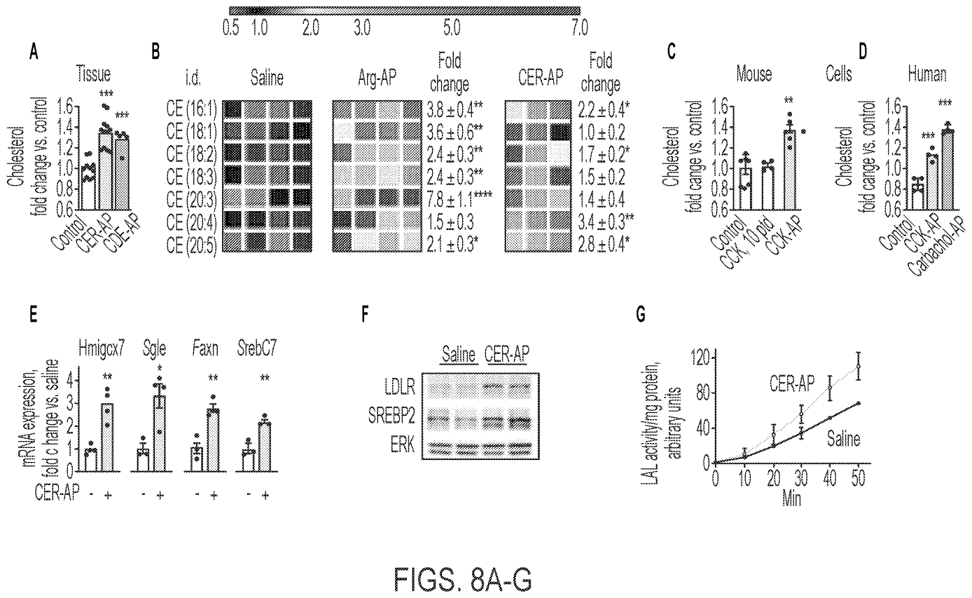

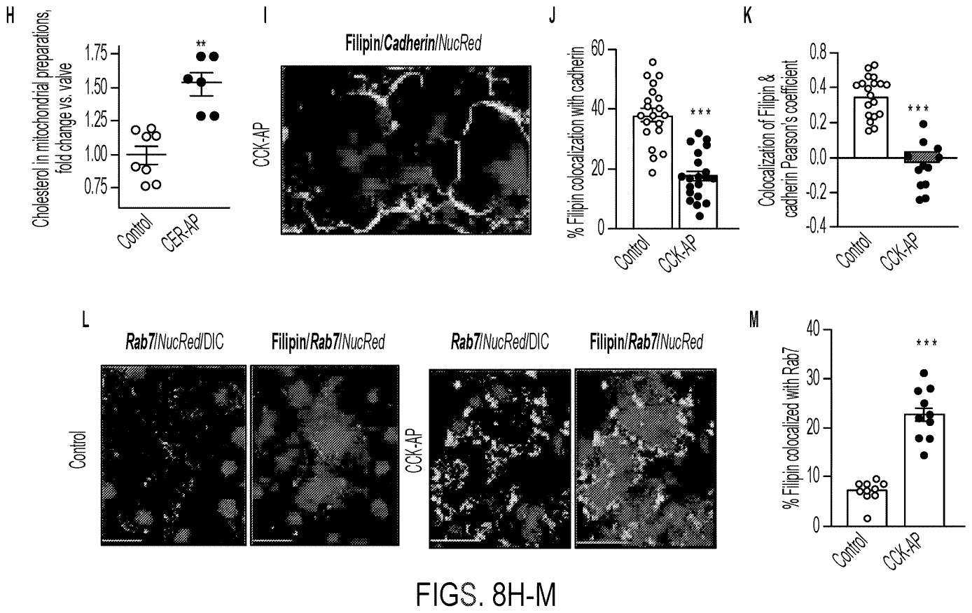

Lipidomic and enzymatic analyses showed increases in pancreatic total cholesterol in 3 dissimilar acute pancreatitis (AP) models induced in WT mice using L-arginine (Arg-AP), the CCK-8 ortholog cerulein (CER-AP), or choline deficient, ethionine-supplemented diet (CDE-AP) (FIGS. 8, A and B). Similarly, cholesterol content increased in mouse (FIG. 8C) and, notably, human (FIG. 8D) acinar cells stimulated with supramaximal (100 nM) CCK-8 (CCK-AP) or carbachol (ex vivo AP models). Physiologic doses of CCK (10 μM) or carbachol did not alter total cholesterol in acinar cells, and pancreatic triglycerides were unchanged in CER-AP (FIG. 8C, and data not shown).

The changes in acinar cell cholesterol metabolism were examined in more detail in CER/CCK-AP, the most widely used animal and ex vivo pancreatitis model (3). Similar to Gnptab−/− (FIG. 4), in WT mice with CER-AP upregulation of pancreatic mRNA expression for enzymes mediating cholesterol synthesis was found (FIG. 8E), increased protein levels of LDLR and SREBP2 (FIG. 8F) and LAL activity (FIG. 8G), and elevated mitochondrial cholesterol (FIG. 8H). Further, the plasma membrane free cholesterol (i.e., filipin colocalization with cadherin) markedly decreased in the ex vivo CCK-AP model, concomitant with its accumulation in Rab7-positive LE/Ly (FIG. 8, I-M). These changes are similar to those seen in KO pancreas. As in Gnptab−/− mice, serum cholesterol levels did not change in CER-AP.

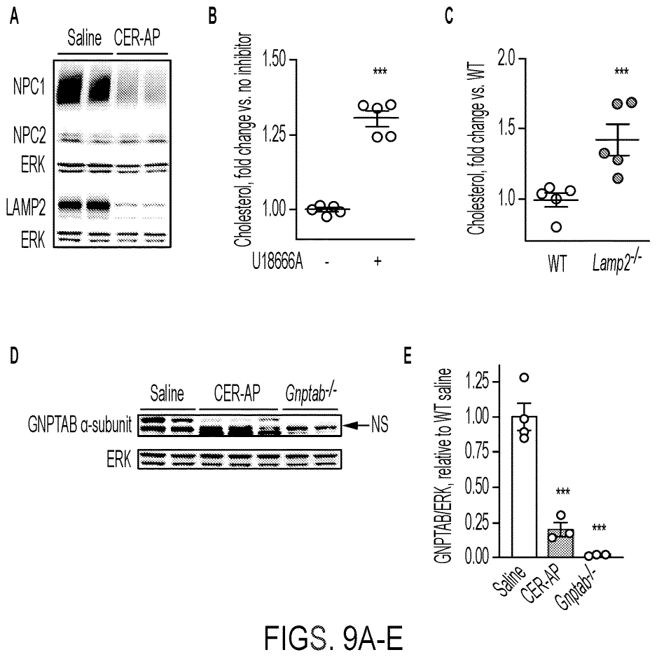

Whether experimental pancreatitis altered the NPC2/NPC1 pathway that mediates cholesterol transport through LE/Ly was tested (39). Unlike Gnptab−/− pancreas, there was no change in NPC2 levels, but pancreatic NPC1 greatly decreased in CER-AP (FIG. 9A). Administration of U18666A, a selective NPC1 inhibitor (57), elevated cholesterol content in WT mouse pancreas (FIG. 9B). A similar increase was found in pancreatic cholesterol in mice deficient in LAMP2 (FIG. 9C), another LE/Ly protein mediating cholesterol trafficking (58). Of note, pancreatic LAMP2 levels greatly decrease in various pancreatitis models, including CER-AP (FIG. 9A), as well as in human disease (8). The results in U18666A-treated WT mice and in Lamp2−/− mice (FIGS. 9, B and C) confirm the role of NPC1 and LAMP2 in regulating pancreatic cholesterol.

CER-AP elicited an approximately 5-fold decrease in pancreatic GNPTAB α-subunit protein level (FIGS. 9, D and E). This effect could mediate alterations in cholesterol metabolism that were observed in experimental pancreatitis. The fact that the NPC2 level was not reduced in CER-AP (FIG. 9A), despite a marked decrease in GNPTAB α-subunit, can indicate increased NPC2 synthesis, blocked excretion, or a threshold pattern of its regulation by the M6P pathway in the pancreas.

viii. Cholesterol Normalization Ameliorates Experimental Pancreatitis

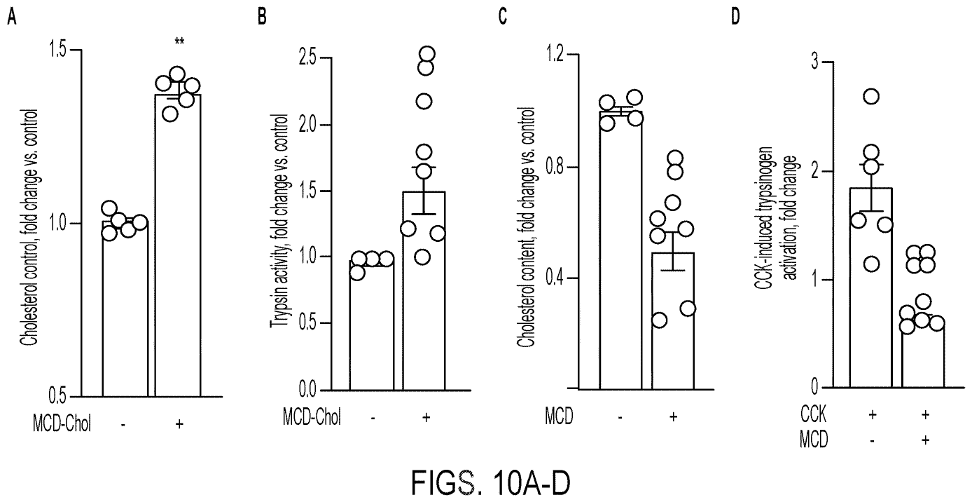

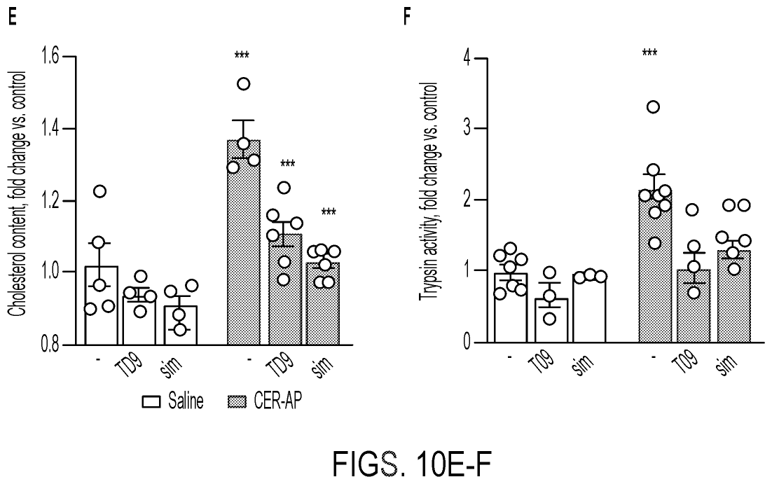

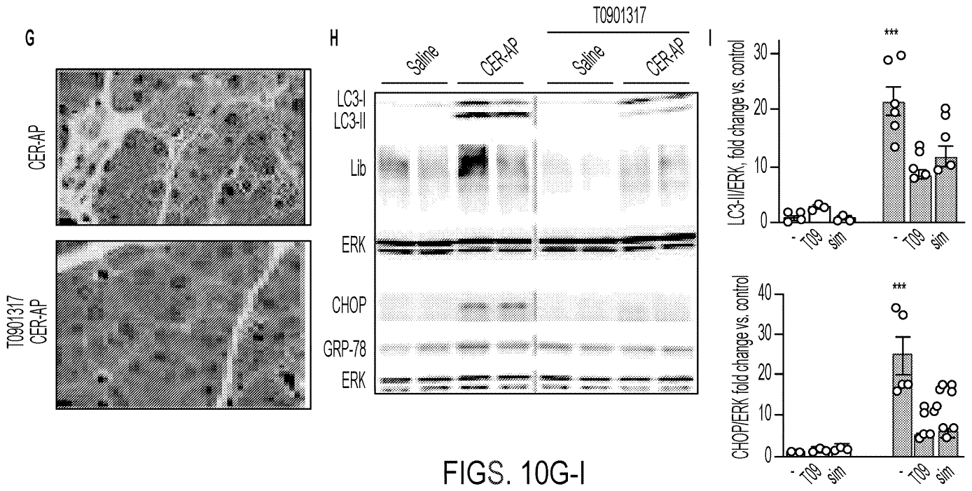

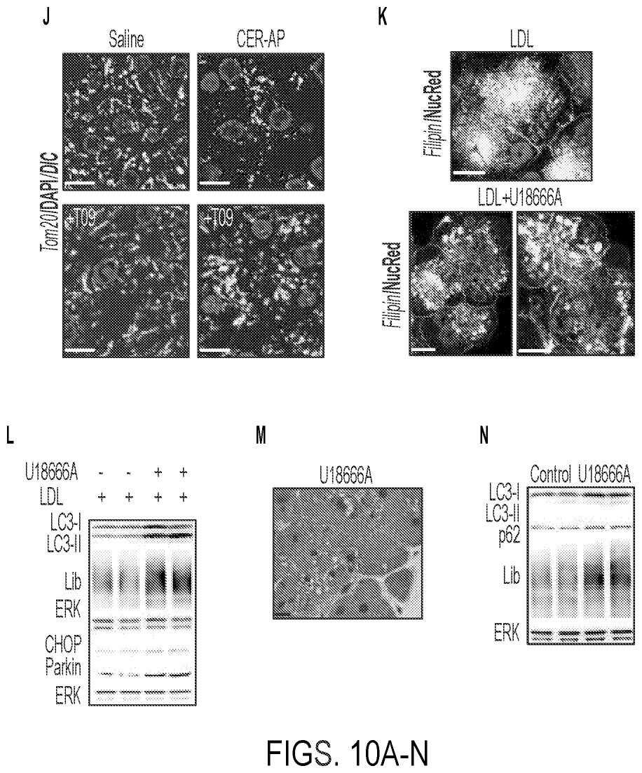

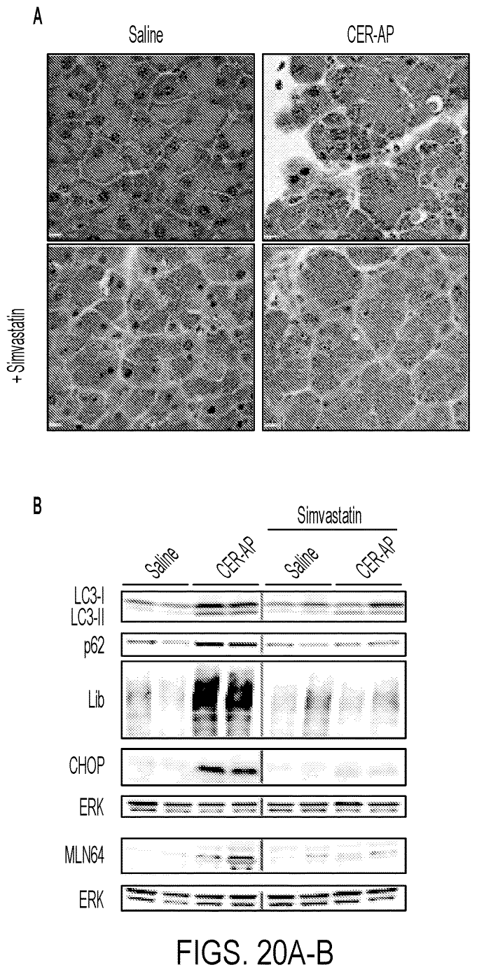

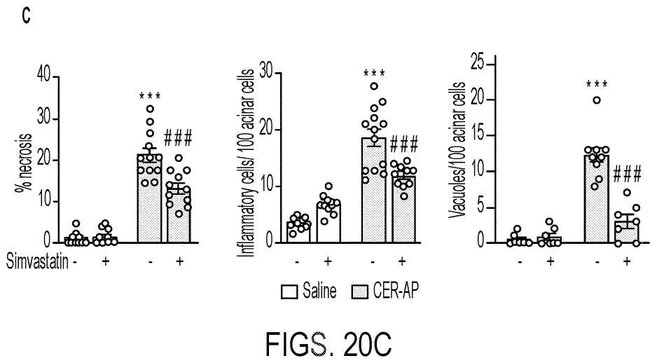

FIG. 10, A-F that, as in Gnptab−/− mice, cholesterol regulates trypsinogen activation in WT experimental pancreatitis. Cholesterol overload (using cholesterol-loaded MCD) increased basal trypsin activity in WT acinar cells (FIGS. 10, A and B), whereas depleting cholesterol with MCD reduced CCK-induced trypsinogen activation in ex vivo pancreatitis (FIGS. 10, C and D). In accord with these data, T0901317, which promotes cholesterol efflux by activating the LXR receptor (59, 60), and simvastatin, an inhibitor of cholesterol synthesis, ameliorated experimental pancreatitis in WT mouse and ex vivo models. Both compounds eliminated pancreatitis-induced increases in total cholesterol and trypsin activity (FIGS. 10, E and F). Of note, the same T0901317 or simvastatin treatments of Gnptab−/− mice, even for 3 weeks, failed to decrease pancreatic total cholesterol levels, confirming the resistance of KO acinar cells to reducing cholesterol overload (see FIGS. 6, Q and R). T0901317 and simvastatin improved pancreas histopathology in CER-AP (FIG. 10G and FIG. 20A); reduced accumulation of LC3-II, p62, and ubiquitylated proteins, indicating more efficient autophagy; and decreased CHOP levels, indicating lower ER stress (FIGS. 10, H and I, and FIG. 20B). T0901317 and simvastatin also normalized mitochondrial fragmentation, as assessed by Tom20 IF (FIG. 10J), and abrogated the CER-induced increase in MLN64 (FIG. 20B). In addition to improved histopathology, simvastatin also ameliorated other pancreatitis responses (FIG. 20C).

To examine the effect of disrupted cholesterol turnover, WT acinar cells were incubated with LDL-cholesterol in the presence and absence of the NPC1 inhibitor U18666A. U18666A caused cholesterol accumulation in LE/Ly (filipin staining; FIG. 10K), resulting in impaired autophagy (manifested by concomitant accumulation of LC3-II and ubiquitylated proteins) and increases in CHOP and Parkin (FIG. 10L). Similarly, mice that received U18666A and thus had elevated total cholesterol (FIG. 9B) exhibited pancreas damage manifested by increased acinar cell vacuolization, necrosis (up to ~20%), and inflammation (FIG. 10M), and accumulation of LC3-II, p62, and ubiquitylated proteins (FIG. 10N).

Taken together, amelioration of experimental pancreatitis with T0901317 and simvastatin, and the detrimental effects of U18666A indicate that disordered acinar cell cholesterol metabolism mediates impaired autophagy, ER stress, and other pathologies of pancreatitis.

ix. Human Pancreatitis Displays Signs of Disordered Acinar Cell Cholesterol Homeostasis

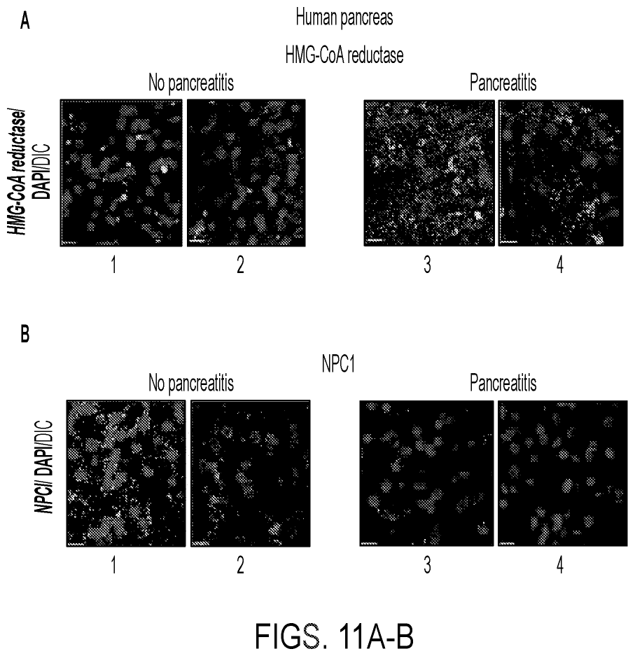

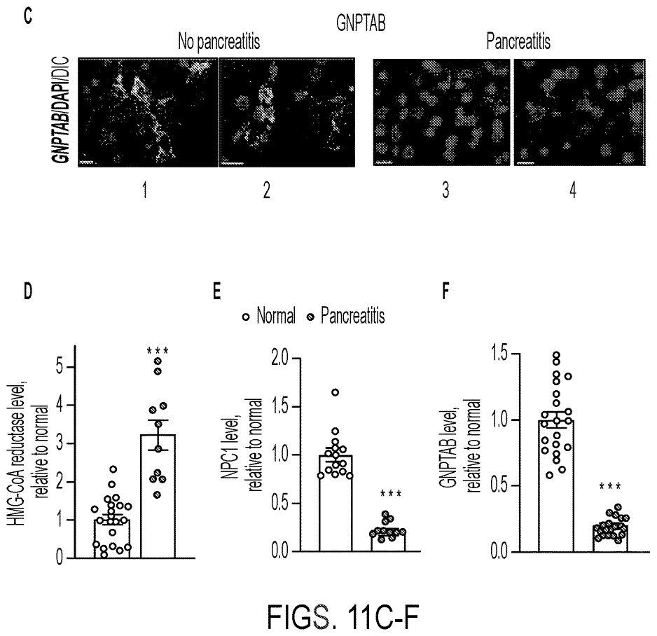

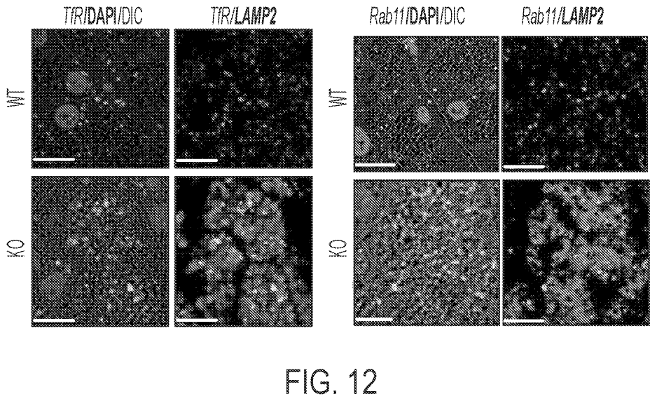

Using IF analysis of deidentified tissue sections from patients with and without pancreatitis, an approximately 3-fold increase in pancreatic HMG-COA reductase, and dramatic decreases in NPC1 and GNPTAB levels in pancreatitis tissues were found (FIG. 11). The specificity of the anti-GNPTAB α-subunit antibody was confirmed by IB analysis (FIG. 9D). None of the patients, except one, had pancreatic adenocarcinoma (Table 1). Remarkably, both the direction and magnitude of the changes observed in human disease are similar to those presented above in experimental models of pancreatitis.

TABLE 1
Human pancreatic tissue

| | | | Diagnosis/Reason | Presumed explanation for |
|---|---|---|---|---|
| Code | Age/Sex | Procedure | for surgery | pancreatitis in sampled section |
| CP1 | 66/F | Whipple | Autoimmune | Autoimmune pancreatitis, |
| | | | pancreatitis, type 2 | type 2 |
| CP2 | 73/F | Enucleation | Well differentiated | Pancreatitis associated with |
| | | | Pancreatic | obstruction caused by PNET |
| CP3 | 61/M | Whipple | Chronic pancreatitis | Chronic pancreatitis of unclear |
| | | | (unclear etiology) | etiology (no support for |
| | | | | autoimmune) |
| CP4 | 71/M | Whipple | Chronic pancreatitis | Chronic pancreatitis of unclear |
| | | | (unclear etiology) | etiology (no support for |
| | | | presenting as mass | autoimmune) |
| CP5 | 21/F | Distal | Chronic pancreatitis | Chronic pancreatitis with duct |
| | | pancreatectomy | with duct stricture | stricture, unclear etiology (no |
| | | | (unclear etiology) | support for autoimmune) |
| CP6 | 69/F | Distal | Low grade IPMN | Pancreatitis associated with |
| | | pancreatectomy | | obstruction caused by IPMN |
| CP7 | 46/M | Whipple | Chronic pancreatitis | Chronic pancreatitis of unclear |
| | | | (unclear etiology) | etiology (no support for |
| | | | | autoimmune) |
| CP8 | 67/M | Distal | Benign congenital | Pancreatitis associated with |
| | | pancreatectomy | cyst | obstruction caused by benign |
| | | | | congenital cyst |
| CP9 | 75/F | Whipple | Well differentiated | Pancreatitis associated with |
| | | | Pancreatic | obstruction caused by PNET |
| | | | Neuroendocrine | |
| | | | Tumor, Grade1 | |
| CP10 | 59/M | Whipple | Chronic pancreatitis | Chronic pancreatitis of unclear |
| | | | (unclear etiology) | etiology (no support for |
| | | | | autoimmune) |
| CP11 | 71/M | Whipple | Moderately | Pancreatitis associated with |
| | | | differentiated | obstruction caused by PDAC |

No pancreatitis tissue

| | | | Diagnosis/Reason | |
|---|---|---|---|---|
| Code | Age/Sex | Procedure | for surgery | Sampled section |
| NP1 | 60/M | Whipple | Ampullary | Uninvolved histologically |
| | | | adenocarcinoma | normal pancreas |
| NP2 | 80/M | Whipple | Ampullary | Uninvolved histologically |
| | | | adenocarcinoma | normal pancreas |
| NP3 | 40/F | Whipple | Well differentiated | Uninvolved histologically |
| | | | Pancreatic | normal pancreas |
| | | | Neuroendocrine | |
| | | | Tumor, Grade 1 | |
| NP4 | 68/M | Distal | Well differentiated | Uninvolved histologically |
| | | pancreatectomy | Pancreatic | normal pancreas |
| | | | Neuroendocrine | |
| | | | Tumor, Grade 1 | |
| NP5 | 51/M | Whipple | Well differentiated | Uninvolved histologically |
| | | | Pancreatic | normal pancreas |
| | | | Neuroendocrine | |
| | | | Tumor, Grade 1 | |
| NP6 | 39/F | Whipple | Well differentiated | Uninvolved histologically |
| | | | Pancreatic | normal pancreas |
| | | | Neuroendocrine | |
| | | | Tumor, Grade 1 | |
| NP7 | 67/M | Distal | Well differentiated | Uninvolved histologically |
| | | pancreatectomy | Pancreatic | normal pancreas |
| | | | Neuroendocrine | |
| | | | Tumor, Grade 1 | |
| NP8 | 40/M | Whipple | Well differentiated | Uninvolved histologically |
| | | | Pancreatic | normal pancreas |
| | | | Neuroendocrine | |
| | | | Tumor, Grade 1 | |
| NP9 | 41/M | Distal | Well differentiated | Uninvolved histologically |
| | | pancreatectomy | Pancreatic | normal pancreas |
| | | | Neuroendocrine | |
| | | | Tumor, Grade 2 | |
| NP10 | 71/M | Whipple | Well differentiated | Uninvolved histologically |
| | | | Pancreatic | normal pancreas |
| | | | Neuroendocrine | |
| | | | Tumor, Grade 2 | |

All samples were taken >1 cm away from any associated lesion. All samples were evaluated and placed in formalin fixative at the time of intraoperative evaluation.

### 3. Discussion

The study in Gnptab−/− mice reveals that an efficient endolysosomal system and autophagy, and in particular the M6P pathway, are critical for maintaining cholesterol metabolism in the exocrine pancreas. Cholesterol derangement mediates experimental pancreatitis pathologies, and the human disease displays signs of impaired acinar cell cholesterol homeostasis. The results show a central role of the M6P pathway in key exocrine pancreas cellular processes: autophagy, endocytosis, and mitochondrial functions; and establish Gnptab−/− mice as a genetic model of pancreatitis.

Gnptab ablation perturbed the acinar cell endolysosomal system, manifested by underprocessing of cathepsins and the expansion of LE/Ly that was not matched by an increase in cathepsins' activities, resulting in diminished lysosomal proteolytic capacity. Impaired EE-to-LE maturation were found causing accumulation of hybrid endosomes. In turn, the major functions of the endolysosomal system, autophagy and endocytic recycling, were severely compromised. Gnptab deficiency both stimulated autophagosome formation in acinar cells and blocked autophagic flux, causing accumulation of large nondegradative autolysosomes. Deranged endocytosis was manifested by increased Tf internalization and inhibition of its recycling.

Gnptab deficiency greatly perturbed many aspects of cholesterol metabolism, as reflected by several-fold increases in total and free cholesterol, upregulated expression of proteins mediating cholesterol synthesis, and increased LDL uptake. In contrast to WT, free cholesterol in KO acinar cells accumulated in large LAMP2-positive structures occupying the bulk of the cytoplasm. This was associated with a deleterious cholesterol reduction in the plasma membrane and zymogen granules. Of note, the distribution of cholesterol among subcellular membranes is highly uneven. In healthy cells, most of cellular cholesterol is localized to the plasma membrane to maintain its proper fluidity and rigidity, as well as to secretory granules; by contrast, mitochondria and lysosomes contain minor amounts of cholesterol (23-26).

The results indicate that two major mechanisms mediating these effects are defective cholesterol trafficking through LE/Ly and blockage of autophagic flux. Autophagy/lipophagy plays a key role in lipid droplet degradation in normal pancreas, and its impairment results in accumulation of neutral lipids in nondegradative autolysosomes, making free cholesterol unavailable for the cell's needs. In response, Gnptab−/− acinar cells upregulate both cholesterol synthesis and uptake, which exacerbates cholesterol derangement by further increasing its content.

This paradoxical response, and other alterations in cholesterol metabolism in Gnptab−/− pancreas, resemble those observed in the lysosomal storage disease NPC. The disease is caused by mutations in NPC2 or NPC1, proteins that mediate cholesterol transport through LE/Ly (39, 58). NPC2 lacking the M6P recognition marker fails to reach lysosomes and is secreted (28, 40). Congruently, NPC2 levels greatly decreased in Gnptab−/− pancreas, likely mediating cholesterol accumulation in LE/Ly. Mouse NPC models showed structural alterations in pancreatic acinar cells, e.g., lipid accumulation in lysosomes and a reduction in secretory granules (61, 62); pancreas damage in NPC patients has not been examined. However, there have been no reports of pancreatitis in NPC patients (or mouse models), indicating that NPC2 deficiency by itself is insufficient to cause disease.

Gnptab ablation caused pancreatic mitochondrial failure evidenced by decreased ETC complex activities and oxidative phosphorylation (OXPHOS), impaired ATP synthesis, and increased mitochondrial fragmentation. The results reveal a critical role for the M6P pathway in maintaining mitochondrial functions in the pancreas. The data implicate several mechanisms mediating mitochondrial failure in Gnptab−/− pancreas. One is the blockage of autophagy/mitophagy, causing accumulation of dysfunctional mitochondria. Another is perturbed neutral lipid metabolism, resulting in impaired β-oxidation and increased mitochondrial cholesterol. The latter can impede ETC complex formation and ATP synthesis (24, 54).

In contrast to the pancreas, Gnptab−/− livers did not exhibit pathologic changes in the endolysosomal system, autophagy, mitochondrial functions, or cholesterol content. This is consistent with a previous study showing organ-specific effects of Gnptab ablation (19), likely due to varying contributions of compensatory, M6P-independent mechanisms of acid hydrolases' targeting to endolysosomes (28). However, some effects were shared by KO pancreas and liver, such as the markedly decreased levels of perilipin 2, a critical lipid droplet protein (38), revealing a role for the M6P pathway in lipid droplet homeostasis.

In humans, mutations in the GNPTAB gene cause mucolipidosis type II (MLII), a fatal disease characterized by damage to mesenchymal cells (i.e., skin fibroblasts) and skeletal and other abnormalities (15, 16, 61). Some, but not all, of these defects are observed in mice with an MLII patient's GNPTAB mutation knocked in (15). Gnptab−/− mice, in which the gene is completely ablated by retroviral gene trapping, do not replicate MLII pathology (18, 19, 28); in particular, there is not much damage to fibroblasts. However, these mice provide a valuable tool to study the roles and mechanisms of endolysosomal and autophagy pathways in secretory cells (this study and ref. 19). Pancreatitis has not been reported in MLII patients, but an autopsy study of juvenile MLII patient (63) found extensive damage (i.e., massive vacuolization) in several secretory glands including exocrine pancreas. The remarkably variable sensitivity of different organs and cell types to defects in GNPTAB, observed in both MLII patients and mouse models (15, 16, 19, 28), reflects the contribution of alternative and compensatory, M6P-independent mechanisms (as well as interspecies differences). The difference between the effects of a mutated (15) versus fully ablated Gnptab gene (this study and refs. 18, 19) can also indicate a role for protein-protein interactions involving GNPTAB, in addition to its enzymatic function. Of note, the ablation of the cation-independent M6P receptor in mice (64) caused much less pancreas damage than in Gnptab−/− (though it worsened experimental pancreatitis), possibly indicating a compensatory contribution of the second, cation-dependent, M6P receptor.

Gnptab−/− mice spontaneously develop pancreatitis with all key manifestations of human disease. Together with recent studies (8-11, 65), the results support the hypothesis (5) that secretory cells, such as pancreatic acinar cells, rely heavily on efficient endolysosomal functions and autophagy and are particularly sensitive to their impairment. In particular, the basal secretion was significantly decreased in Gnptab−/− acinar cells. In addition, a recent study (66) showed that efficient lysosomal proteolytic degradation is required for cellular reprogramming into a regenerative phenotype and that Gnptab deficiency profoundly impairs the pancreas's ability to regenerate after CER-induced injury.

Prompted by the results in Gnptab−/−, experimental pancreatitis in WT mouse and ex vivo models greatly perturbed acinar cell cholesterol metabolism. Similar to Gnptab−/− pancreas, total cholesterol content was elevated in several unrelated acute pancreatitis models. Experimental pancreatitis caused cholesterol accumulation in LE/Ly and autolysosomes, reduced free cholesterol in the plasma membrane, and increased the expression of proteins mediating cholesterol synthesis, LDLR levels, and mitochondrial cholesterol. Both Gnptab ablation and experimental pancreatitis disrupted cholesterol transport through LE/Ly but by different mechanisms; the former reduced the NPC2 level, while the latter decreased NPC1 (as well as LAMP2 [ref. 8], another LE/Ly cholesterol transporter).

Cholesterol is involved in the regulation of intra-acinar trypsinogen activation, a signature pancreatitis response, the mechanism of which remains obscure (6, 43, 67, 68). Trypsin activity increased with cholesterol overload, while in both Gnptab−/− acinar cells and WT experimental pancreatitis it was reduced by decreasing cholesterol content with different approaches. Inhibiting cholesterol turnover with U18666A caused impaired autophagy and ER stress in acinar cells, whereas alleviating cholesterol overload with T090137 or simvastatin reduced these and other pathologic responses of experimental pancreatitis. Simvastatin also abrogated the increase in pancreatic MLN64 caused by CER-AP. Collectively, the results reveal the critical role of cholesterol in maintaining acinar cell homeostasis and implicate its derangement in pancreatitis development.

The finding of manifestations of disordered acinar cell cholesterol homeostasis in human pancreatitis supports the conclusions from mouse and cellular models. In particular, the reduction in NPC1, together with previous finding of a dramatic LAMP2 decrease in human pancreatitis (8), strongly indicate deranged cholesterol trafficking through LE/Ly in human disease. The decrease in pancreatic GNPTAB levels, observed in both human disease and an experimental model, further justifies Gnptab−/− mice as a genetic pancreatitis model.

Dysregulation of acinar cell cholesterol is a previously unrecognized pathology of pancreatitis. In fact, very little is known about basal cholesterol metabolism in the exocrine pancreas, the maintenance of which should be critical for acinar cells, as zymogen granule formation, secretion, and membrane recycling/retrieval all require precise and adequate lipid supply. One likely reason for this lack of knowledge is that human pancreatitis has not been associated with changes in blood cholesterol. This disconnect is reminiscent of NPC patients who have normal serum cholesterol levels despite fatal defects in cholesterol metabolism in many organs (69, 70). Likewise, changes in serum cholesterol were not observed in either Gnptab−/− or experimental pancreatitis.

However, there have been epidemiologic studies on the effects of statins on pancreatitis severity in patients, with contradictory findings (71-74), and a clinical trial (NCT02743364) is ongoing on simvastatin's effect in alleviating pancreatitis. The premise of the above studies (as well as a recent study in a mouse pancreatitis model; ref. 75) is that statins may alter pancreatitis severity through off-target effects, particularly on inflammation; changes in pancreatic cholesterol were not investigated in patients with pancreatitis. However, the prevailing opinion is shifting toward recognizing statins' potential for pharmacologic prevention of pancreatitis (74). Cholesterol dyshomeostasis is increasingly recognized as a key player in other multifactorial diseases, such as neurodegenerative (e.g., Alzheimer's) diseases, in which it is considered a novel target for treatment (76). This study provides evidence for categorizing pancreatitis as a cholesterol disorder disease and elucidates the mechanisms of the effects of cholesterol-lowering drugs in pancreatitis.

### 4. Methods

i. Antibodies.

Antibodies against the following proteins were purchased from Cell Signaling Technology: ATG12 (catalog 4180), Beclin1 (catalog 3495), ATG7 (catalog 2631), CHOP (catalog 5554), LC3B (catalog 2775), SQSTMI/p62 (catalog 5114), phospho-NF-κB p65/Ser536 (catalog 3033), p44/42 MAPK (ERK1/2; catalog 4696 and 9102), phospho-STAT3/Tyr705 (catalog 9145), and STAT3 (catalog 9139). Antibodies against the following proteins were purchased from Abcam: CD3 (catalog ab119332), F4/80 (catalog ab6640), LAMP1 (catalog ab24170), Rab5 (catalog ab18211), Rab9 (catalog ab2810), and SREBP2 (catalog ab30682). Antibodies against the following proteins were purchased from Santa Cruz Biotechnology: EEA1 (catalog sc-137130), LAMP1 (catalog sc-17768), LDLR (catalog sc-18823), NF-κB p65 (catalog sc-8008), trypsinogen (catalog sc-137077), and ubiquitin (catalog sc-8017). Antibodies against the following proteins were purchased from Sigma-Aldrich: α-amylase (catalog A8273), LAMP2 (catalog L0668), and Rab7 (catalog R8779). Antibodies against other proteins were purchased from the following companies: cadherin (Novus, catalog NB200-59), EPG5 (Byorbyt, catalog orb323262), perilipins 2 and 5 (ProGen, catalog GP40 and GP31), perilipin 3 (ProSci, catalog 3881), myeloperoxidase (Thermo Fisher Scientific, catalog PA5-16672), and MLN64/STARD3 (Invitrogen, catalog PAS-76266).

ii. Reagents.

CellLight Early Endosomes-GFP and Late Endosomes-RFP BacMam 2.0 were from Thermo Fisher Scientific (catalog C10586 and C10589, respectively); Cholesterol and Triglyceride Assay kits, from Abcam (catalog ab65390 and ab65336); HR series NEFA-HR2 kits to measure free fatty acids, from Wako Diagnostics (catalog 991-34891); LDL Uptake and ALT Assay kits, from Abcam (catalog ab133127 and ab105134); and AST Assay kit, from Sigma-Aldrich (catalog MAK055).

Experimental pancreatitis models in mice were performed as described previously (8, 31, 46, 48, 77) and detailed in the supplemental material. All the experiments were performed on mice of both sexes if not stated otherwise.

iii. Genetically Modified Mice.

Gnptab−/− (18, 19) and Lamp2−/− mice (8, 78) have been described. Atg5fl/− mice, obtained from Masaki Ohmuraya (Department of Genetics, Hyogo College of Medicine, Nishinomiya, Hyogo, Japan) (79), were crossed with Pdx1-Cre driver mice to generate Atg5fl/−; Pdx1-Cre mice (termed Atg5Δpan) in which the Atg5 gene is specifically ablated in pancreatic epithelial cells. Control mice were littermates with all WT alleles. All these strains of mice are on the C57BL/6 genetic background and have been bred for at least 10 generations.

iv. Human Pancreas Specimens.

Formalin-fixed, paraffin-embedded samples of human normal pancreas and pancreatitis (see supplemental material) were deidentified and provided by the Pancreas Tissue Bank of the UCLA Department of Pathology. Tissue sections were evaluated for quality and the presence of an acinar compartment (8, 46).

Human pancreas slices (80) were obtained as detailed herein.

Ex vivo pancreatitis models were performed on mouse and human acinar cells, and human pancreas slices by incubation with supramaximal CCK (100 nM) or carbachol (1 mM), as described previously (80, 81).

v. Lipidomics.

Unbiased complex lipid analysis of pancreatic samples was performed by the NIH West Coast Metabolomics Center (UC Davis) using ultra-high-pressure liquid chromatography and tandem mass spectrometry.

vi. Statistics.

Statistical analysis of the results was performed with Prism 8 (GraphPad Software) using a 2-tailed Student's t test for comparison between 2 groups and 1-way ANOVA with Tukey's post hoc test for comparison between multiple groups. Values are expressed as mean±SEM; a P value of less than 0.05 was considered significant. Statistical details for each experiment, including n values, are provided in figure legends.

vii. Experimental Pancreatitis in Mice

Cerulein-induced (CER-AP) and L-arginine induced (Arg-AP) acute pancreatitis models were performed in 12-week-old C57BL/6 mice that received 7 hourly i.p. injections of 50 μg/kg cerulein or 3 hourly i.p. injections of 3.3 g/kg Arg; controls received physiologic saline injections. Mice were euthanized for analyses 7 hours after the first cerulein or 24 hours after the first Arg injection. Choline deficient, ethionine-supplemented (CDE) pancreatitis was induced in young (~5-weeks old) female mice fed either CDE or control diet. Mice were euthanized 72 hours after the diet initiation. These models have been described in detail (1-7).

viii. In Vivo Treatments of Mice

12-week-old WT mice were subjected for 4 consecutive days to daily i.p. injections of 20 mg/kg simvastatin (8,9) or 20 mg/kg T0901317 (10); or 10 mg/kg U18666A for 15 days (11). Control mice received vehicle injections. Some of the simvastatin- and T0901317-treated mice were subjected to CER-AP on day 4, as indicated in corresponding figures.

12-week-old Gnptab−/− mice were subjected to daily i.p. injections of 20 mg/kg simvastatin or the same dose of T0901317 for 4 or 21 days. Neither treatment changed total cholesterol content in pancreas.

ix. Pancreas Subcellular Fractionation

Subcellular fractionation of pancreatic tissue was performed by differential centrifugation (7). Pancreata were collected and homogenized in 8 ml of homogenization buffer with 5 full strokes in a Dounce homogenizer, and the nuclei and cell debris sedimented at 150 g. The postnuclear supernatant was centrifuged at 1,300 g, and the pellet collected and referred to as fraction Z. The supernatant was further centrifuged at 12,000 g, and both the 12,000-g pellet (fraction L) and supernatant (fraction C) were collected. Total protein in the fractions was measured by Bradford assay (Bio-Rad). The amounts of total protein in each fraction were consistent across experimental conditions. The quality of subcellular fractionation was evaluated by IB analysis for specific protein markers [this study and Ref. (7)].

x. Isolation of Mouse Pancreatic Acinar Cells and Lobules

Pancreatic acinar cells were isolated from mice of both sexes using a standard collagenase digestion procedure (1-6). Pancreatic lobules (~2-mm thick) were excised and incubated at 37° C. in 199 medium containing 0.1 mg/mL soybean trypsin inhibitor (1).

xi. Human Pancreas Slices

Human pancreas slices were obtained as in (12), from portions of live normal human pancreata from pre-terminal donors that were not used for transplantation and diverted to research (provided by Trillium Gift of Life Network, Toronto, Ontario, year 2020) or from normal portions of pancreatic cancer operations. Pancreas was cut into ~3×3×3 mm pieces and embedded with 37° C. low melting 3.8% agarose gel in extracellular solution containing (mM): 125 NaCl, 2.5 KCl, 1 MgCl2, 2 CaCl2, 1.25 NaH2PO4, 26 NaHCO3, 2 sodium pyruvate, 0.25 ascorbic acid, 3 myo-inositol, 6 lactic acid, and 7 glucose, and placed on ice to solidify the gel. The gelled tissue blocks were sliced with Vibratome into 120 μm-thick slices; slices were washed with the same extracellular solution, placed in 199 medium containing 0.1 mg/mL soybean trypsin inhibitor, and treated with CCK-8 (CCK) or carbachol for ex-vivo pancreatitis.

xii. Baculoviral Transduction

CellLightTM Rab5a-GFP and Rab7a-RFP reagents were added to suspension of isolated mouse acinar cells to reach the final concentration of 30 particles/cell, per manufacturer's protocol. The cells were gently shaken at 37° C. for 1 h to maximize transduction efficiency, cultured for another 16 hours, fixed, and analyzed by confocal microscopy.

xiii. Mitochondria Isolation and Functional Assays

Mitochondria were isolated from mouse pancreas and liver in a Ca2+-free medium in the presence of 1 mM EGTA, as in (3-5,13). The value of respiratory control ratio in these preparations, measured in the presence of succinate (quality control parameter), was greater than 3.0.

Mitochondrial membrane potential (Δ′-Pm) was measured in mitochondria suspension (1 mg/ml protein) using a tetraphenyl phosphonium (TPP+) electrode (3-5,13) in a buffer containing (mM) 250 sucrose, 22 KCl, 22 triethanolamine (pH 7.4), 3 MgCl2, 5 KH2PO4, 1 EGTA, supplemented with 0.5% BSA and 10 mM glutamate plus 2 mM malate as the respiratory substrate. An increase in Δ′-Pm results in TPP+ uptake by mitochondria and, correspondingly, a decrease in external TPP+ concentration measured with the TPP+ electrode. The mitochondrial uncoupler FCCP was added at the end of each experiment to cancel Δ′-Pm and thus normalize the changes in Δ′-Pm. Variations in the basal Δ′-Pm values between mitochondria preparations from normal mouse pancreas did not exceed 3%.

Specific activities of individual ETC complexes were measured spectrophotometrically in homogenates of isolated mitochondria or submitochondrial particles, as in (14).

Oxygen consumption rates (OCR) were measured in live acinar cells using Seahorse XFe96, as in (15). As shown in FIG. 7, the decrease in OCR upon injection of the ATP synthase inhibitor oligomycin represents a portion of basal respiration that is used to drive ATP production (ATP-linked). Addition of the uncoupler FCCP, which stimulates the respiratory chain to operate at maximum capacity, yields the value of maximal OCR. The combination of complex I inhibitor rotenone and complex III inhibitor antimycin A is added to shut down mitochondrial respiration, providing the contribution of non-mitochondrial respiration to correct for. In all within-set experiments the same volume of cell suspension were used with the same amount of protein; and partial oxygen pressure was monitored to ensure its stability in the cell suspension.

xiv. IB Analysis was Done as in (1-7,15)

Immunoblots were developed using enhanced chemiluminescence detection kit (Pierce), and band intensities were quantified by densitometry using FluorChem HD2 imaging system (Alpha Innotech/ProteinSimple).

xv. Fluorescence and Light Microscopy were Utilized as Described in (1-7,15)

Pancreatic tissue was cut into ~3×3×3 mm pieces and fixed overnight at 4° C. with 4% (w/v) methanol-free paraformaldehyde in 0.1 M phosphate buffer. Tissue pieces were then equilibrated in 15% sucrose-phosphate buffer, embedded in Tissue-Tek OCT compound, and stored at-80° C. For paraffin embedding, pancreatic tissue pieces were fixed in 10% buffered formaldehyde. Acinar cell pellets were fixed for 20 min at room temperature in 4% (w/v) paraformaldehyde in 0.1 M phosphate buffer.

Fixed-frozen or paraffin-embedded pancreas and liver tissue sections and fixed cells were processed as in (1-7) and immunolabeled by incubating overnight at 4° C. with primary antibodies, followed by incubation with secondary antibodies conjugated with FITC or Texas Red. Nuclei were counterstained with DAPI or NucRed 647.

Staining for neutral lipids and free cholesterol was performed by incubation of fixed-frozen tissue sections or fixed cells with, respectively, 2 μg/ml BODIPY493/503 for 45 min or 100 □g/ml filipin for 2 h at room temperature. For double labeling, BODIPY493/503 or filipin were applied after fixed-frozen tissue or cell sections incubated with primary and secondary antibodies.

To label acidic organelles, acinar cells were incubated with 100 nM LysoTracker Deep Red for 15 min at RT, washed, and fixed for 20 min in 4% (w/v) paraformaldehyde in 0.1 M phosphate buffer at room temperature.

For trypsinogen activation peptide (TAP) immunolabeling, pancreatic lobules were fixed with 2% paraformaldehyde and 0.05% glutaraldehyde in PBS, and processed as in (7,16).

All fluorescence images were acquired with a Zeiss LSM710 confocal microscope using x63 objective and preset within-experiment image acquisition settings. Quantitative fluorescence analysis was performed with Volocity software (PerkinElmer). Overlap between 2 fluorescent labels was measured using Manders-Costes coefficient. Pearson's colocalization coefficient was calculated if both immunolabeled proteins had the same (e.g. “dotted”) staining pattern.

Immunohistochemical detection of the infiltrating inflammatory cells was performed on paraffin-embedded pancreas sections using streptavidin-biotin immunoenzymatic antigen detection system, as in (1,2).

xvi. Transmission Electron Microscopy

For electron microscopy (1-3,7), the tissue was cut into 1-mm cubes and fixed overnight at 4° C. in 2.5% glutaraldehyde, 150 mM sodium cacodylate (pH 7.4). After postfixation in 1% OsO4 followed by uranyl acetate, the tissue was dehydrated in ethanol and embedded in epoxy resin. 100-nm-thick sections were examined (S.W.F.) in a Hitachi-600 electron microscope.

qPCR. Total RNA was isolated from tissue or cells using QIAGEN RNeasy Plus kit according to the manufacturer's instructions, quantified in NanoDrop, and converted to cDNA with high-capacity cDNA reverse transcription kit. KAPA SYBR FAST qPCR Master Mix kit and a LightCycler 480 were used for quantitative PCR. Fold change related to the control group was calculated using the 2ΔΔCt method with Rplp0/36B4 as the reference gene. Primers are available upon request.

xvii. Transferrin-Alexa594 (Tf-594) Internalization Assay

Mouse acinar cells were incubated for 2 h in serum-free 199 medium, pulsed with the fluorescently labeled Tf-594 (25 □g/ml) for 30 min on ice, washed with PBS, transferred to 37° C., and chased for 3, 30, and 90 min. At these time points, cells were placed on ice, fixed, and analyzed with confocal microscopy.

xviii. Measurement of Protease and Lipase Activities

Activities of cathepsin (Cat) B and L, caspase-3, trypsin, and lysosomal acid lipase (LAL) were measured in pancreatic tissue or cell homogenates and subcellular fractions with fluorogenic assays using substrates specific for CatB (Z-Arg-Arg-AMC), CatL (Z-Phe-Arg-AMC), caspase-3-like (Z-VAD-fmk), trypsin (Boc-Gln-Ala-Arg-AMC), as previously described (1-4, 6, 7); and for LAL (4-methylumbelliferyl oleate), as in (17). In measurements of CatL activity, the assay buffer also contained 50 μM CA-074me, a CatB inhibitor. LAL activity was measured in the absence and presence of the LAL inhibitor lalistat (100 μM) as the difference between these values (17).

xix. Cholesterol and Triglyceride Assays

Total cholesterol content was measured with Cholesterol Assay kit per manufacturer's instructions. Samples of mouse pancreas and liver, cell pellet homogenates, subcellular fractions, isolated mitochondria, and serum were diluted in the reaction buffer containing cholesterol probe and incubated at 37° C. for 1 h in the dark. To measure total cholesterol, the samples were subjected to digestion with cholesterol esterase. To measure non-esterified cholesterol, the esterase was omitted from the assay.

Triglycerides (TAG) content was measured using Triglyceride Assay kit. Tissue samples were homogenized in 10% (w/v) NP-40 aqueous solution, subjected to two heating/cooling cycles to solubilize triglycerides, and centrifuged to remove insoluble material. Supernatants were diluted with water and mixed with lipase-containing reaction mix per manufacturer's instructions.

For measurement of free fatty acids (FFA), lipids were extracted from tissue or isolated mitochondria using chloroform:methanol (2:1 v/v). FFA content was measured by enzymatic assay using HR kits.

Fluorescence intensity (for cholesterol) and absorbance (for TAG) were measured in a Tecan microplate reader. Each measurement was in duplicate, and the values were normalized per mg total protein in the sample.

xx. LDL Uptake

LDL uptake was measured as in (18). Isolated acinar cells were incubated in serum-free 199 medium for 2 h at RT, washed with PBS, and incubated with 5 μg/mL DyLight550-labeled LDL for additional 2 h at 37° C. Then, cells were resuspended in a lysis buffer; cell debris were removed by centrifugation, and the fluorescent signal in cell lysate was measured in a Typhoon imager at Ex/Em=540/570 nm and normalized to total protein in the lysate.

xxi. Cytokine/Chemokine Measurements

Samples were prepared as in (19). Briefly, tissue was homogenized in the presence of 25 U/ml benzonase endonuclease, and protease inhibitors cocktail and incubated on ice, and cell debris were removed by centrifugation at 4° C. Cytokines and chemokines were measured in the supernatants with Luminex® 200™ Multiplexing Instrument using mouse 32-plex Cytokine/Chemokine panel, and normalized per total protein in the sample.

Pancreatitis responses were measured as previously described (1-7). Serum amylase and lipase were measured in a Hitachi 707 analyzer. Acinar cell vacuolization and necrosis were quantified on H&E stained pancreatic tissue sections; at least 1000 acinar cells/mouse were analyzed in several high-power fields. ATP was measured in tissue homogenates using bioluminescence assay (3-5).

Amylase secretion was assayed as in (1,2), by measuring the amount of amylase both in the incubation medium and in cells with the Phadebas amylase assay kit.

xxii. Serum Markers of Liver Damage

Plasma concentrations of aspartate aminotransferase and alanine aminotransferase were determined by corresponding assay kits per manufacturer's instructions.

### B. Example 2

### 1. Current Status of Research

i. Acute and Chronic Pancreatitis

Pancreatitis is a progressive, potentially fatal, disease of the exocrine pancreas characterized by inflammation, fibrosis, and loss of pancreas functions.^{1, 2} There are 2 major forms of pancreatitis, acute (AP), which usually produces an episode of temporary illness, and chronic (CP), a disease associated with severe pain, poor quality of life, and increased risk for the deadliest pancreatic cancer.^{1, 3} It is believed that CP results from repetitive subclinical or clinically evident bouts of AP or may develop without prior AP. Pancreatitis is the third most common reason for hospital admissions in those with GI disease and a heavy burden on the U.S. healthcare system.^{1, 2} Major AP responses include inappropriate/intra-acinar activation of digestive enzymes, particularly trypsinogen; increased serum levels of amylase; neutrophil-driven inflammatory cell infiltration in the pancreas; and acinar cell death. Key pathologic features of CP are progressive loss of acinar tissue, chronic inflammation, and fibrosis, ultimately leading to loss of exocrine as well as endocrine pancreatic function. It is believed that the disease is initiated by injured acinar cells; there is compelling evidence that mechanisms mediating pancreatitis are common to all forms of the disease.^{1, 2, 4} The pathogenesis of pancreatitis remains obscure, and no effective treatment is available.¹ The reason for that is primarily because the underlying molecular and cellular mechanisms are not understood.

ii. Disordering of Lysosomal/Autophagy Pathways Drives Pancreatitis.

Autophagy is a principal mechanism through which cells eliminate unneeded or damaged components and recycle generated products, such as amino acids and lipids, for critical cellular processes.⁵⁻⁸ In this process, cargo destined for degradation is sequestered by specific organelles, autophagosomes, which then deliver cargo to lysosomes to be degraded by acid hydrolases. Impaired autophagy is a critical feature of both human and experimental pancreatitis.^{5-7, 9-13} It manifests, in particular, by the cell's failure to eliminate damaged mitochondria and ER, and to generate cholesterol by autophagic degradation of lipid droplets.¹³ The cause of impaired autophagy in experimental and human pancreatitis is lysosomal dysfunction. Its major manifestations include decreased levels of major lysosomal membrane protein LAMP2; 10, 11 loss of the master transcriptional regulator of lysosomal biogenesis, TFEB; 12 and decrease in GNPTAB, 13 a key enzyme mediating acid hydrolases' delivery to the lysosome.^{13, 14} Recent studies in genetic mouse models that specifically replicate lysosomal defects of pancreatitis showed that genetic ablation of LAMP2, TFEB, GNPTAB^{10, 12, 13} or block of autophagosome formation^{15, 16} all cause autophagy blockade in acinar cells and development of spontaneous pancreatitis. Conversely, enhancing lysosomal biogenesis with the pharmacologic autophagy enhancer, trehalose, largely prevented pancreas injury in AP models.¹⁷ Although these studies reveal an essential role of impaired lysosomal/autophagy pathways in initiating and driving pancreatitis, how their dysfunction causes disease remains unknown.

A recent study¹³ revealed that lysosomal/autophagy pathways control cholesterol homeostasis in acinar cells and that cholesterol dysregulation, caused by lysosomal/autophagy dysfunction, exacerbates pancreatitis, the results further indicate that normalizing cholesterol metabolism alleviates disease, implicating cholesterol metabolism as a clinically relevant modulator of pancreatitis severity.

iii. Cholesterol Metabolism in Mammalian Cells.

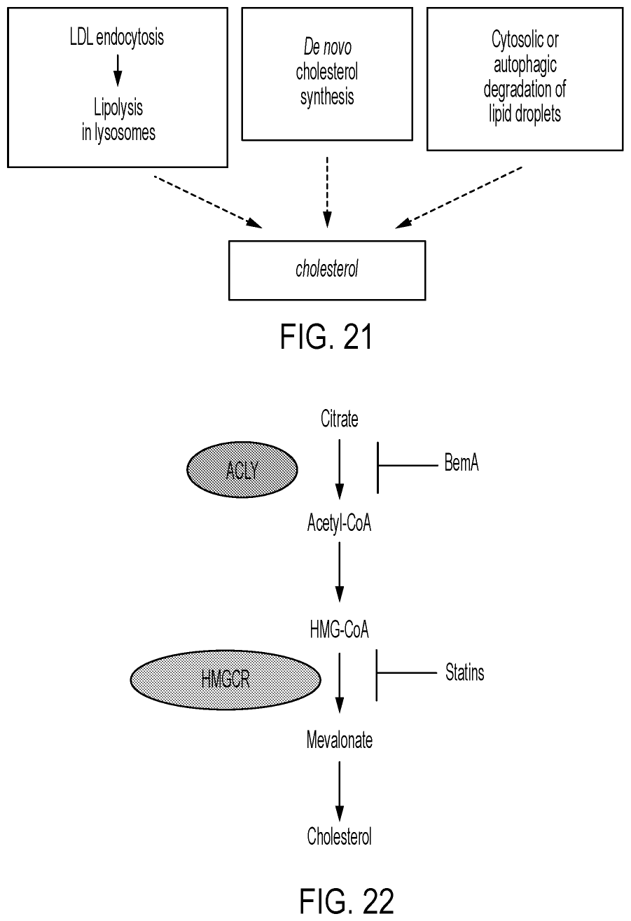

Cholesterol is the principal sterol synthesized by all animals.^{18, 19} Cholesterol comprises about 30% of cell membranes' lipids, and its unique structure enables cells to alter membrane fluidity while preserving membranes' integrity so that they remain stable without becoming rigid.^{18, 19} Maintaining distinct cholesterol levels in the plasma and cytoplasmic organellar membranes is necessary for cell functioning.¹⁸⁻²⁰ Cholesterol exists in cells in esterified and non-esterified (free) forms. Mammalian cells obtain free cholesterol via 4 routes¹⁸⁻²¹ (FIG. 21). Cholesterol carrying low-density lipoproteins (LDL) enter cells through endocytosis and undergo lipolysis in lysosomes, yielding free cholesterol. De novo cholesterol synthesis starts with citrate conversion to acetyl-CoA by ATP-citrate lyase (ACLY),^{22, 23} resulting in generation of HMG-COA which is converted by HMG-COA reductase (HMGCR) to mevalonate^{18, 19} and ultimately cholesterol (FIG. 22). Cholesterol is also produced by lipolysis of lipid droplets that store cholesterol esters, which is mediated by either neutral (cytosolic) lipases or autophagy/lipophagy.¹⁸⁻²¹

Disruption of cellular cholesterol metabolism and elevated blood cholesterol are two major causes of cholesterol dyshomeostasis diseases. 24, 25 Inactivating mutations in NPC1 and NPC2 proteins that mediate cholesterol transport through the lysosome cause fatal Niemann-Pick type C (NPC) disease. Patients with NPC disease display massive cholesterol accumulation in lysosomes and a concomitant reduction of free cholesterol available for cell's needs.^{19, 25, 26} Differently, atherosclerosis is caused by increased blood cholesterol resulting in excessive cholesterol uptake by blood macrophages and leading to their transformation into lipid-laden foam cells, which form atherosclerotic plaque.^{27, 28} Of note, lysosomal cholesterol dysregulation diseases, such as the NPC, occur without a noticeable increase in blood cholesterol.^{29, 30} Importantly, cholesterol dyshomeostasis is increasingly recognized as a key player in multifactorial, especially neurodegenerative diseases (e.g., Alzheimer's), in which it is considered a novel target for treatment.^{25, 31}

Cholesterol-lowering drugs. Statins are the most commonly prescribed cholesterol-lowering drugs, used primarily for the prevention and treatment of cardiovascular diseases.³⁰ They reduce cholesterol synthesis by inhibiting HMG-COA reductase (HMGCR), the rate-limiting enzyme of cholesterol biosynthesis (FIG. 22). Although statins are generally safe, ~10% of patients do not tolerate statin therapy because of its toxicity to muscles.³² Bempedoic acid (BemA), an inhibitor of ATP-citrate lyase (ACLY) that acts upstream of HMGCR (FIG. 2), has been recently approved by FDA as a novel therapy for lowering cholesterol.^{22, 23, 33} BemA is a prodrug activated in the liver, and therefore has minimal myotoxicity making BemA an attractive alternative to statins.

Cholesterol metabolism is disrupted in pancreatitis as a result of lysosomal/autophagy dysfunction. Preventing acinar cell cholesterol overload with simvastatin alleviates AP in one experimental model (CER-AP). As discussed above, lysosomal/autophagy dysfunction is associated with both experimental and human pancreatitis.^{6, 7, 9, 13, 17} However, until a recent publication¹³ there was very little known on cholesterol metabolism in healthy pancreas, its changes in experimental and human pancreatitis, and role in disease severity. Further, effects of statins and other cholesterol-lowering drugs in pancreas, and the underlying mechanisms, have not been explored. The results¹³ showed that disrupted acinar cell cholesterol trafficking in pancreatitis causes its excessive accumulation in lysosomes and a decrease in free cholesterol available for cell's needs. This, in turn, stimulates a compensatory increase in cholesterol synthesis, further exacerbating cholesterol overload. It was further shown¹³ that inhibition of cholesterol synthesis with simvastatin reduced acinar cell cholesterol overload and the severity of pancreatitis induced with supramaximal cerulein (CER), an ortholog of the hormone cholecystokinin-8 (CCK). The findings¹³ reveal a previously unrecognized pathogenic role of cholesterol dysregulation in pancreatitis and indicate normalizing cholesterol metabolism as a novel therapeutic approach for disease treatment.

The links between statins' use and human pancreatitis have been reported for years.³⁴⁻⁴¹ The results of early reports were controversial, likely due to inadequate approaches, and some even suggested worsening of the disease. However, recent case-control, population and cohort studies indicate that statins are associated with a lower risk of AP and decreased mortality.³⁴⁻⁴¹ Of note, none of these studies considered cholesterol dysregulation in acinar cells as a target for statins' impact on pancreatitis.

The overall objective of the project is to validate the role of cholesterol dysregulation in driving pancreatitis and establish cholesterol synthesis pathway as a therapeutic target amenable to pharmacologic intervention in pancreatitis. The effects of two cholesterol-lowering drugs with different mechanisms of action, simvastatin and BemA, were examined on disease severity in several dissimilar models of AP and CP. Validating the impact of cholesterol-lowering drugs on experimental pancreatitis is necessary to de-risk future clinical trials and drug development studies. Repurposing of FDA-approved drugs with known safety profiles, such as simvastatin and BemA, can significantly speed up and lower cost of drug development, and is therefore an attractive strategy for treatment of pancreatitis, addressing an unmet and urgent need.

### 2. Relevance to VA Patient Care Mission

Excessive alcohol consumption is a major cause of both AP and CP; smoking and age are other key risk factors for this disease.^{1, 42} The prevalence of these factors results in high pancreatitis incidence in Veterans⁴³ as well as in military personnel;⁴⁴ in fact, individuals with a history of pancreatitis are, as a rule, disqualified from military service because the onset of pancreatitis is abrupt and very painful, and thus can significantly impair the military mission capabilities of afflicted.⁴⁴ Veterans with pancreatitis have a 3-fold greater risk of pancreatic cancer compared to the non-Veteran patient population.⁴³

### 3. Significance

This study can validate the use of cholesterol-lowering drugs simvastatin and BemA for the treatment of pancreatitis, the disease for which pain medications and iv fluids is the only treatment currently available. Both drugs are FDA-approved and widely used for the prevention and treatment of cardiovascular diseases. Drug repurposing offers advantages over developing a new drug because the time and cost of the drug development could be significantly reduced.45 Therapeutic potential of simvastatin and BemA can be explored by using different regimens in several clinically relevant AP and CP models. The results allow for selecting the best candidate(s) for clinical trials by excluding conditions with toxic effects on acinar cells, insufficient cholesterol-lowering capacity, and/or little beneficial effect on disease parameters.

### 4. Results

The overall goal of this project is to validate the hypothesis that pancreatitis is a cholesterol disorder disease and that cholesterol synthesis pathway is a therapeutic target amenable to pharmacologic intervention in pancreatitis. This study can comparatively examine the effects of cholesterol-lowering drugs, simvastatin^{30, 46} and BemA,^{22, 23, 33} on disease severity and pancreas cholesterol level in several clinically relevant mouse AP and CP models and ex-vivo models on mouse and human acinar cells.

The disruption of lysosomal and autophagy pathways in pancreatitis is established by multiple studies.^{5-7, 9-13, 47-49} The findings on the central role of

Cholesterol dysregulation in driving pancreatitis, recently published in The Journal of Clinical Investigation,¹³ are based on the analysis of genetic and experimental mouse models, human pancreatitis, and ex-vivo pancreatitis in mouse and human acinar cells. Multiple methodologic approaches, namely LC-MS/GC-MS lipidomic analysis, enzymatic assays, immunoblot (IB), immunofluorecence (IF; confocal microscopy), qPCR, were used to validate the conclusions. The studies were performed on mice of both sexes, and the data were categorized according to sex.

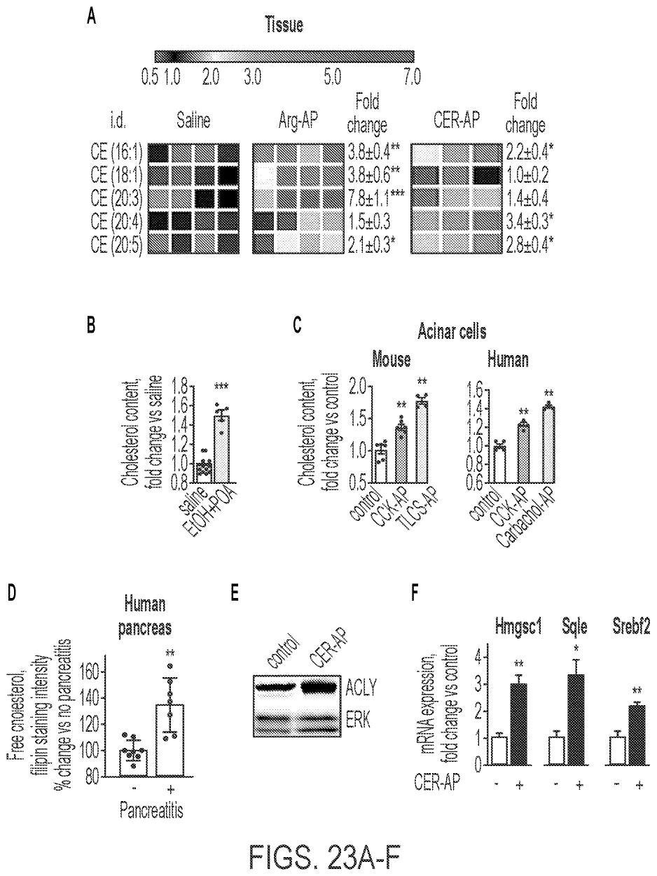

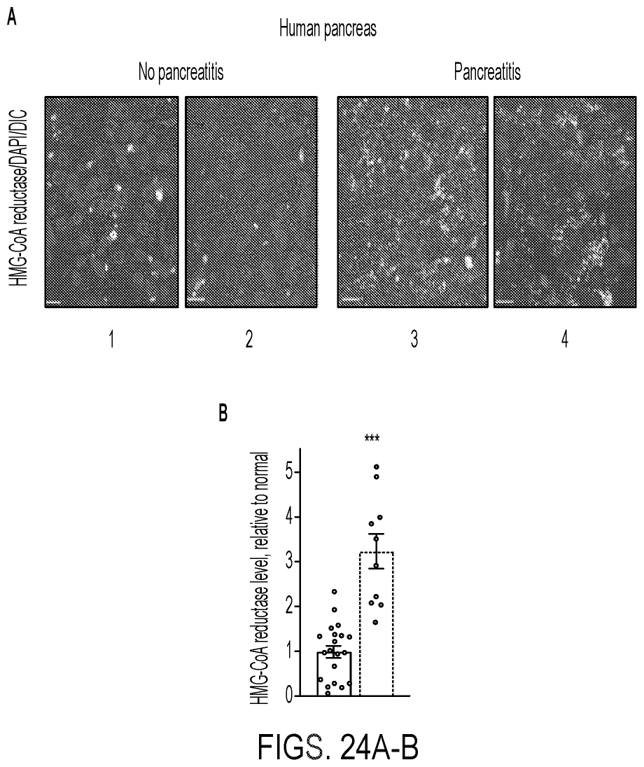

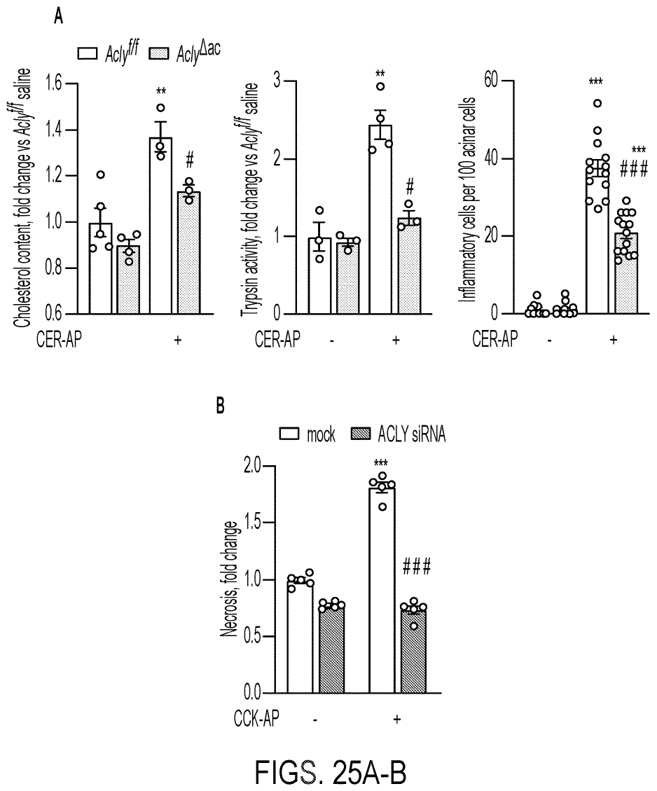

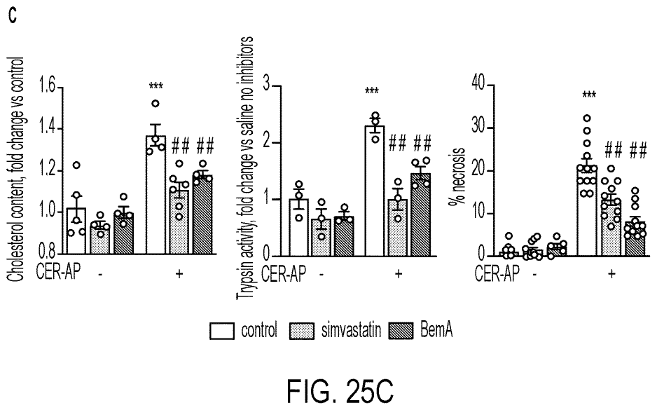

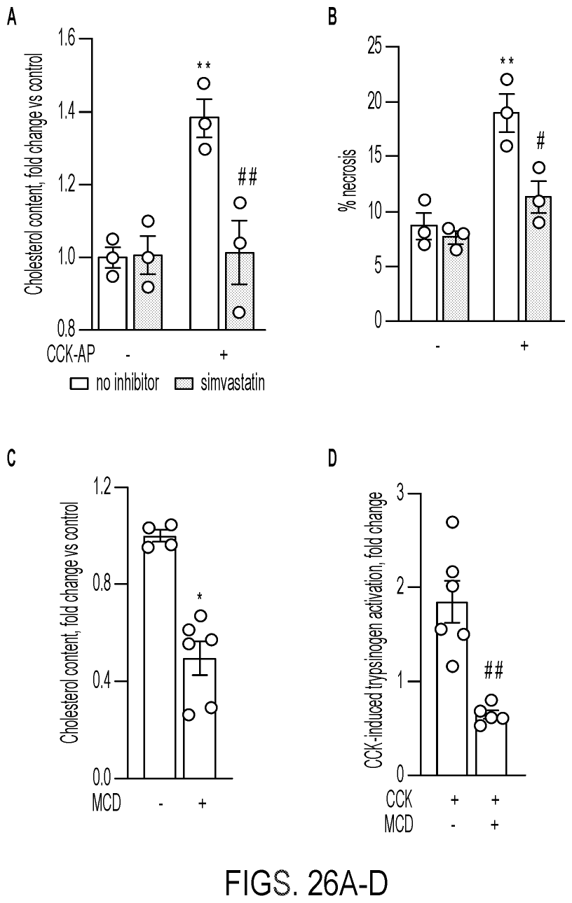

Cholesterol metabolism is profoundly perturbed in experimental and human pancreatitis (FIG. 23 and Ref.13). Detailed lipidomic analysis using LS-MS/GS-MS (FIG. 23A) and enzymatic assays (FIG. 23B,C) revealed greatly elevated pancreatic cholesterol levels in several dissimilar mouse AP models; as well as upregulated mRNA or protein expression of enzymes involved in cholesterol synthesis: ACLY, HMGCR (coded by the Hmgscl gene), squalene monooxygenase (Sqle), and the key transcription factor Sterol regulatory element-binding protein 2 (Srebf2) (FIG. 23E,F). Acinar cells cholesterol overload was prominent in ex-vivo pancreatitis induced by CCK, TLCS or carbachol in mouse and, importantly, human acinar cells (FIG. 23C). Further, tissue sections from pancreatitis patients displayed elevated free cholesterol levels (FIG. 23D) as visualized with its' specific probe filipin.⁵⁰

Pathways mediating acinar cell cholesterol homeostasis are dysregulated in both human and experimental pancreatitis (FIG. 24 and Refs.10, 11, 13), manifested by decreases in the levels of cholesterol transporters through lysosomes, NPC113 and LAMP2, 10, 11 and dramatic HMGCR upregulation (FIGS. 23F and 24).

Inhibiting cholesterol synthesis by genetic, molecular or pharmacologic approaches reduces the severity of experimental AP (FIG. 25 and Ref.13). Acinar cell-specific genetic ablation of Acly (FIG. 25A); ACLY siRNA knockdown (FIG. 25B); or the inhibitors of cholesterol synthesis, simvastatin and BemA (FIG. 25C), all decreased cholesterol overload and ameliorated major pathologic responses of CER-AP: trypsin activity, inflammation, and necrosis.

Preventing acinar cell cholesterol overload with different approaches alleviates ex-vivo pancreatitis (FIG. 26 and Ref.13). Reducing cholesterol overload with simvastatin or cholesterol chelator methyl cyclodextrin (MCD) greatly decreased the extent of necrosis and trypsinogen activation in ex-vivo CCK-AP (FIG. 26).

In summary, the published¹³ and current data (FIGS. 23-26) reveal that disruption of acinar cell cholesterol metabolism is a common feature in models of experimental pancreatitis and also prominent in human disease. The key manifestation of cholesterol dyshomeostasis in pancreatitis is acinar cell cholesterol overload. Inhibition of cholesterol synthesis with simvastatin and BemA prevented cholesterol overload and reduced disease severity in one particular mouse and ex-vivo model (CER/CCK-AP). These findings indicated cholesterol-lowering drugs as a promising approach for pancreatitis treatment and became the basis for the present proposal.

Those skilled in the art will recognize, or be able to ascertain using no more than routine experimentation, many equivalents to the specific embodiments of the method and compositions described herein. Such equivalents are intended to be encompassed by the following claims.

### EXAMPLE 1 REFERENCES

1. Pandol S J, et al. Acute pancreatitis: bench to the bedside. Gastroenterology. 2007; 132 (3): 1056-1151. Erratum: ibid, 133 (3): 1056. [PubMed] [Google Scholar]
2. Peery A F, et al. Burden and cost of gastrointestinal, liver, and pancreatic diseases in the United States: update 2018. Gastroenterology. 2019; 156 (1): 254-272. doi: 10.1053/j.gastro.2018.08.063. [PMC free article] [PubMed] [CrossRef] [Google Scholar]
3. Lerch M M, Gorelick F S. Models of acute and chronic pancreatitis. Gastroenterology. 2013; 144 (6): 1180-1193. doi: 10.1053/j.gastro.2012.12.043. [PubMed] [CrossRef][Google Scholar]
4. Maléth J, et al. Alcohol disrupts levels and function of the cystic fibrosis transmembrane conductance regulator to promote development of pancreatitis. Gastroenterology. 2015; 148 (2): 427-439. doi: 10.1053/j.gastro.2014.11.002. [PMC free article] [PubMed] [CrossRef] [Google Scholar]
5. Gukovskaya A S, et al. Recent insights into the pathogenic mechanism of pancreatitis: role of acinar cell organelle disorders. Pancreas. 2019; 48 (4): 459-470. doi: 10.1097/MPA.0000000000001298. [PMC free article] [PubMed] [CrossRef] [Google Scholar]
6. Mareninova O A, et al. Impaired autophagic flux mediates acinar cell vacuole formation and trypsinogen activation in rodent models of acute pancreatitis. J Clin Invest. 2009; 119 (11): 3340-3355. doi: 10.1172/JCI38674. [PMC free article] [PubMed] [CrossRef] [Google Scholar]
7. Habtezion A, et al. Acute pancreatitis: a multi-faceted set of organellar, cellular and organ interactions. Gastroenterology. 2019; 156 (7): 1941-1950. doi: 10.1053/j.gastro.2018.11.082. [PMC free article] [PubMed] [CrossRef] [Google Scholar]
8. Mareninova O A, et al. Lysosome associated membrane proteins maintain pancreatic acinar cell homeostasis: LAMP-2 deficient mice develop pancreatitis. Cell Mol Gastroenterol Hepatol. 2015; 1 (6): 678-694. doi: 10.1016/j.jcmgh.2015.07.006. [PMC free article] [PubMed] [CrossRef][Google Scholar]
9. Diakopoulos K N, et al. Impaired autophagy induces chronic atrophic pancreatitis in mice via sex- and nutrition-dependent processes. Gastroenterology. 2015; 148 (3): 626-638. doi: 10.1053/j.gastro.2014.12.003. [PubMed] [CrossRef] [Google Scholar]
10. Antonucci L, et al. Basal autophagy maintains pancreatic acinar cell homeostasis and protein synthesis and prevents ER stress. Proc Natl Acad Sci USA. 2015; 112 (45): E6166-E6174. doi: 10.1073/pnas. 1519384112. [PMC free article] [PubMed] [CrossRef] [Google Scholar]
11. Wang S, et al. Impaired TFEB-mediated lysosomal biogenesis promotes the development of pancreatitis in mice and is associated with human pancreatitis. Autophagy. 2019; 15 (11): 1954-1969. doi: 10.1080/15548627.2019.1596486. [PMC free article] [PubMed] [CrossRef][Google Scholar]
12. Kornfeld S. Trafficking of lysosomal enzymes in normal and disease states. J Clin Invest. 1986; 77 (1): 1-6. doi: 10.1172/JCI112262. [PMC free article] [PubMed] [CrossRef][Google Scholar]
13. Ghosh P, et al. Mannose 6-phosphate receptors: new twists in the tale. Nat Rev Mol Cell Biol. 2003; 4 (3): 202-212. doi: 10.1038/nrm1050. [PubMed] [CrossRef] [Google Scholar]
14. Braulke T, Bonifacino J S. Sorting of lysosomal proteins. Biochim Biophys Acta. 2009; 1793 (4): 605-614. [PubMed] [Google Scholar]
15. Koehne T, et al. Mannose 6-phosphate-dependent targeting of lysosomal enzymes is required for normal craniofacial and dental development. Biochim Biophys Acta. 2016; 1862 (9): 1570-1580. [PubMed] [Google Scholar]
16. Coutinho M F, et al. Mannose-6-phosphate pathway: a review on its role in lysosomal function and dysfunction. Mol Genet Metab. 2012; 105 (4): 542-550. doi: 10.1016/j.ymgme.2011.12.012. [PubMed] [CrossRef] [Google Scholar]
17. Tiede S, et al. Mucolipidosis II is caused by mutations in GNPTA encoding the alpha/beta GlcNAc-1-phosphotransferase. Nat Med. 2005; 11 (10): 1109-1112. doi: 10.1038/nm1305. [PubMed] [CrossRef] [Google Scholar]
18. Gelfman C M, et al. Mice lacking alpha/beta subunits of GlcNAc-1-phosphotransferase exhibit growth retardation, retinal degeneration, and secretory cell lesions. Invest Ophthalmol Vis Sci. 2007; 48 (11): 5221-5228. doi: 10.1167/iovs.07-0452. [PubMed] [CrossRef][Google Scholar]
19. Boonen M, et al. Vacuolization of mucolipidosis type II mouse exocrine gland cells represents accumulation of autolysosomes. Mol Biol Cell. 2011; 22 (8): 1135-1147. doi: 10.1091/mbc.e10-07-0584. [PMC free article] [PubMed] [CrossRef] [Google Scholar]
20. Helin H, et al. Pancreatic acinar ultrastructure in human acute pancreatitis. Virchows Arch A Pathol Anat Histol. 1980; 387 (3): 259-270. doi: 10.1007/BF00454829. [PubMed] [CrossRef][Google Scholar]
21. Aho H J, et al. Human acute pancreatitis: a light and electron microscopic study. Acta Pathol Microbiol Immunol Scand A. 1982; 90 (5): 367-373. [PubMed] [Google Scholar]
22. Gukovskaya A S, Gukovsky I. Autophagy and pancreatitis. Am J Physiol Gastrointest Liver Physiol. 2012; 303 (9): G993-G1003. doi: 10.1152/ajpgi.00122.2012. [PMC free article] [PubMed] [CrossRef] [Google Scholar]
23. Ikonen E. Cellular cholesterol trafficking and compartmentalization. Nat Rev Mol Cell Biol. 2008; 9 (2): 125-138. doi: 10.1038/nrm2336. [PubMed] [CrossRef] [Google Scholar]
24. Martin L A, et al. Mitochondrial cholesterol: mechanisms of import and effects on mitochondrial function. J Bioenerg Biomembr. 2016; 48 (2): 137-151. doi: 10.1007/s10863-014-9592-6. [PubMed] [CrossRef] [Google Scholar]
25. Maxfield F R, van Meer G. Cholesterol, the central lipid of mammalian cells. Curr Opin Cell Biol. 2010; 22 (4): 422-429. doi: 10.1016/j.ceb.2010.05.004. [PMC free article] [PubMed] [CrossRef] [Google Scholar]
26. Mesmin B, et al. Insights into the mechanisms of sterol transport between organelles. Cell Mol Life Sci. 2013; 70 (18): 3405-3421. doi: 10.1007/s00018-012-1247-3. [PubMed] [CrossRef][Google Scholar]
27. Rowan A D, et al. Rat procathepsin B. Proteolytic processing to the mature form in vitro. J Biol Chem. 1992; 267 (22): 15993-15999. doi: 10.1016/S0021-9258 (19) 49632-4. [PubMed] [CrossRef] [Google Scholar]
28. Markmann S, et al. Quantitative proteome analysis of mouse liver lysosomes provides evidence for mannose 6-phosphate-independent targeting mechanisms of acid hydrolases in mucolipidosis II. Mol Cell Proteomics. 2017; 16 (3): 438-450. doi: 10.1074/mcp.M116.063636. [PMC free article] [PubMed] [CrossRef] [Google Scholar]
29. Tartakoff A M, Jamieson J D. Subcellular fractionation of the pancreas. Methods Enzymol. 1974; 31:41-59. [PubMed] [Google Scholar]
30. Saluja A, et al. Subcellular redistribution of lysosomal enzymes during caerulein-induced pancreatitis. Am J Physiol. 1987; 253 (4 pt 1): G508-G516. [PubMed] [Google Scholar]
31. Mareninova O A, et al. Transgenic expression of GFP-LC3 perturbs autophagy in exocrine pancreas and acute pancreatitis responses in mice. Autophagy. 2020; 16 (11): 2084-2097. doi: 10.1080/15548627.2020.1715047. [PMC free article] [PubMed] [CrossRef] [Google Scholar]
32. Klionsky D J, et al. Guidelines for the use and interpretation of assays for monitoring autophagy (4th edition) 1. Autophagy. 2021; 17 (1): 1-382. doi: 10.1080/15548627.2020.1797280. [PMC free article] [PubMed] [CrossRef] [Google Scholar]
33. Zhao H, et al. Mice deficient in Epg5 exhibit selective neuronal vulnerability to degeneration. J Cell Biol. 2013; 200 (6): 731-741. doi: 10.1083/jcb.201211014. [PMC free article] [PubMed] [CrossRef] [Google Scholar]
34. Haas A K, et al. A GTPase-activating protein controls Rab5 function in endocytic trafficking. Nat Cell Biol. 2005; 7 (9): 887-893. doi: 10.1038/ncb1290. [PubMed] [CrossRef][Google Scholar]
35. Hsu V W, et al. Getting active: protein sorting in endocytic recycling. Nat Rev Mol Cell Biol. 2012; 13 (5): 323-328. doi: 10.1038/nrm3332. [PubMed] [CrossRef] [Google Scholar]
36. Baumann J M, et al. Metabolic assays for detection of neutral fat stores. Bio Protoc. 2015; 5 (12): e1511. [PMC free article] [PubMed] [Google Scholar]
37. Singh R, et al. Autophagy regulates lipid metabolism. Nature. 2009; 458 (7242): 1131-1135. doi: 10.1038/nature07976. [PMC free article] [PubMed] [CrossRef] [Google Scholar]
38. Sztalryd C, Brasaemle D L. The perilipin family of lipid droplet proteins: Gatekeepers of intracellular lipolysis. Biochim Biophys Acta Mol Cell Biol Lipids. 2017; 1862 (10 pt B): 1221-1232. [PMC free article] [PubMed] [Google Scholar]
39. Garver W S, Heidenreich R A. The Niemann-Pick C proteins and trafficking of cholesterol through the late endosomal/lysosomal system. Curr Mol Med. 2002; 2 (5): 485-505. doi: 10.2174/1566524023362375. [PubMed] [CrossRef] [Google Scholar]
40. Willenborg M, et al. Mannose 6-phosphate receptors, Niemann-Pick C2 protein, and lysosomal cholesterol accumulation. J Lipid Res. 2005; 46 (12): 2559-2569. doi: 10.1194/jlr.M500131-JLR200. [PubMed] [CrossRef] [Google Scholar]
41. Schmidt K, et al. Regulated apical secretion of zymogens in rat pancreas. Involvement of the glycosylphosphatidylinositol-anchored glycoprotein GP-2, the lectin ZG16p, and cholesterol-glycosphingolipid-enriched microdomains. J Biol Chem. 2001; 276 (17): 14315-14323. doi: 10.1074/jbc.M006221200. [PubMed] [CrossRef] [Google Scholar]
42. Otani T, et al. Codistribution of TAP and the granule membrane protein GRAMP-92 in rat caerulein-induced pancreatitis. Am J Physiol. 1998; 275 (5): G999-G1009. [PubMed][Google Scholar]
43. Saluja A K, et al. Why does pancreatic overstimulation cause pancreatitis? Annu Rev Physiol. 2007; 69:249-269. doi: 10.1146/annurev.physiol.69.031905.161253. [PubMed] [CrossRef] [Google Scholar]
44. Halangk W, et al. Role of cathepsin B in intracellular trypsinogen activation and the onset of acute pancreatitis. J Clin Invest. 2000; 106 (6): 773-781. doi: 10.1172/JCI9411. [PMC free article] [PubMed] [CrossRef] [Google Scholar]
45. Mahammad S, Parmryd I. Cholesterol depletion using methyl-β-cyclodextrin. Methods Mol Biol. 2015; 1232:91-102. doi: 10.1007/978-1-4939-1752-5_8. [PubMed] [CrossRef][Google Scholar]
46. Biczo G, et al. Mitochondrial dysfunction, through impaired autophagy, leads to endoplasmic reticulum stress, deregulated lipid metabolism, and pancreatitis in animal models. Gastroenterology. 2018; 154 (3): 689-703. doi: 10.1053/j.gastro.2017.10.012. [PMC free article] [PubMed] [CrossRef] [Google Scholar]
47. Mukherjee R, et al. Mechanism of mitochondrial permeability transition pore induction and damage in the pancreas: inhibition prevents acute pancreatitis by protecting production of ATP. Gut. 2016; 65 (8): 1333-1346. doi: 10.1136/gutjnl-2014-308553. [PMC free article] [PubMed] [CrossRef] [Google Scholar]
48. Shalbueva N, et al. Effects of oxidative alcohol metabolism on the mitochondrial permeability transition pore and necrosis in a mouse model of alcoholic pancreatitis. Gastroenterology. 2013; 144 (2): 437-446. doi: 10.1053/j.gastro.2012.10.037. [PMC free article] [PubMed] [CrossRef] [Google Scholar]
49. Yoo S M, Jung Y K. A molecular approach to mitophagy and mitochondrial dynamics. Mol Cells. 2018; 41 (1): 18-26. [PMC free article] [PubMed] [Google Scholar]
50. Qu Q, et al. Fatty acid oxidation and carnitine palmitoyltransferase I: emerging therapeutic targets in cancer. Cell Death Dis. 2016; 7: e2226. [PMC free article] [PubMed] [Google Scholar]
51. Aon M A, et al. Mitochondrial and cellular mechanisms for managing lipid excess. Front Physiol. 2014; 5:282. [PMC free article] [PubMed] [Google Scholar]
52. Su X, et al. Accumulation of long-chain acylcarnitine and 3-hydroxy acylcarnitine molecular species in diabetic myocardium: identification of alterations in mitochondrial fatty acid processing in diabetic myocardium by shotgun lipidomics. Biochemistry. 2005; 44 (13): 5234-5245. doi: 10.1021/bi047773a. [PubMed] [CrossRef] [Google Scholar]
53. McCann M R, et al. L-carnitine and acylcarnitines: mitochondrial biomarkers for precision medicine. Metabolites. 2021; 11 (1): 51. doi: 10.3390/metabo11010051. [PMC free article] [PubMed] [CrossRef] [Google Scholar]
54. Solsona-Vilarrasa E, et al. Cholesterol enrichment in liver mitochondria impairs oxidative phosphorylation and disrupts the assembly of respiratory supercomplexes. Redox Biol. 2019; 24:101214. [PMC free article] [PubMed] [Google Scholar]
55. Charman M, et al. MLN64 mediates egress of cholesterol from endosomes to mitochondria in the absence of functional Niemann-Pick Type C1 protein. J Lipid Res. 2010; 51 (5): 1023-1034. doi: 10.1194/jlr.M002345. [PMC free article] [PubMed] [CrossRef] [Google Scholar]
56. Balboa E, et al. MLN64 induces mitochondrial dysfunction associated with increased mitochondrial cholesterol content. Redox Biol. 2017; 12:274-284. doi: 10.1016/j.redox.2017.02.024. [PMC free article] [PubMed] [CrossRef] [Google Scholar]
57. Liscum L, Faust J R. The intracellular transport of low density lipoprotein-derived cholesterol is inhibited in Chinese hamster ovary cells cultured with 3-beta-[2-(diethylamino) ethoxy] androst-5-en-17-one. J Biol Chem. 1989; 264 (20): 11796-11806. doi: 10.1016/S0021-9258 (18) 80136-3. [PubMed] [CrossRef] [Google Scholar]
58. Schneede A, et al. Role for LAMP-2 in endosomal cholesterol transport. J Cell Mol Med. 2011; 15 (2): 280-295. doi: 10.1111/j.1582-4934.2009.00973.x. [PMC free article] [PubMed] [CrossRef] [Google Scholar]
59. Hong C, Tontonoz P. Liver X receptors in lipid metabolism: opportunities for drug discovery. Nat Rev Drug Discov. 2014; 13 (6): 433-444. doi: 10.1038/nrd4280. [PubMed] [CrossRef] [Google Scholar]
60. Kashiwagi K, et al. Expression of liver X receptors in normal and refractory carcinoma tissues of the human lung and pancreas. Histol Histopathol. 2018; 33 (5): 497-505. [PubMed][Google Scholar]
61. Hammel I, Alroy J. The effect of lysosomal storage diseases on secretory cells: an ultrastructural study of pancreas as an example. J Submicrosc Cytol Pathol. 1995; 27 (2): 143-160. [PubMed] [Google Scholar]
62. Weintraub H, et al. Morphometric studies of pancreatic acinar granule formation in NCTR-BALB/c mice. J Cell Sci. 1992; 102 (pt 1): 141-147. [PubMed] [Google Scholar]
63. Elleder M, Martin J J. Mucolipidosis type II with evidence of a novel storage site. Virchows Arch. 1998; 433 (6): 575-578. doi: 10.1007/s004280050292. [PubMed] [CrossRef][Google Scholar]
64. Meister T, et al. Missorting of cathepsin B into the secretory compartment of CI-MPR/IGFII-deficient mice does not induce spontaneous trypsinogen activation but leads to enhanced trypsin activity during experimental pancreatitis-without affecting disease severity. J Physiol Pharmacol. 2010; 61 (5): 565-575. [PubMed] [Google Scholar]
65. Li N, et al. Loss of acinar cell IKKa triggers spontaneous pancreatitis in mice. J Clin Invest. 2013; 123 (5): 2231-2243. doi: 10.1172/JCI64498. [PMC free article] [PubMed] [CrossRef][Google Scholar]
66. Willet S G, et al. Regenerative proliferation of differentiated cells by mTORC1-dependent paligenosis. EMBO J. 2018; 37 (7): e98311. [PMC free article] [PubMed] [Google Scholar]
67. De Faveri F, et al. LAP-like non-canonical autophagy and evolution of endocytic vacuoles in pancreatic acinar cells. Autophagy. 2020; 16 (7): 1314-1331. doi: 10.1080/15548627.2019.1679514. [PMC free article] [PubMed] [CrossRef] [Google Scholar]
68. Sendler M, Lerch M M. The complex role of trypsin in pancreatitis. Gastroenterology. 2020; 158 (4): 822-826. doi: 10.1053/j.gastro.2019.12.025. [PubMed] [CrossRef][Google Scholar]
69. Vanier M T, Latour P. Laboratory diagnosis of Niemann-Pick disease type C: the filipin staining test. Methods Cell Biol. 2015; 126:357-375. doi: 10.1016/bs.mcb.2014.10.028. [PubMed] [CrossRef] [Google Scholar]
70. Cluzeau C V, et al. Microarray expression analysis and identification of serum biomarkers for Niemann-Pick disease, type C1. Hum Mol Genet. 2012; 21 (16): 3632-3646. doi: 10.1093/hmg/dds193. [PMC free article] [PubMed] [CrossRef] [Google Scholar]
71. Kuoppala J, et al. Use of statins and the risk of acute pancreatitis: a population-based case-control study. Pharmacoepidemiol Drug Saf. 2015; 24 (10): 1085-1092. doi: 10.1002/pds.3858. [PubMed] [CrossRef] [Google Scholar]
72. Lin C M, et al. Use of simvastatin and risk of acute pancreatitis: a nationwide case-control study in Taiwan. J Clin Pharmacol. 2017; 57 (7): 918-923. doi: 10.1002/jcph.881. [PubMed] [CrossRef] [Google Scholar]
73. Wu B U, et al. Simvastatin is associated with reduced risk of acute pancreatitis: findings from a regional integrated healthcare system. Gut. 2015; 64 (1): 133-138. doi: 10.1136/gutjnl-2013-306564. [PMC free article] [PubMed] [CrossRef] [Google Scholar]
74. Machicado J D, Papachristou G I. Pharmacologic management and prevention of acute pancreatitis. Curr Opin Gastroenterol. 2019; 35 (5): 460-467. doi: 10.1097/MOG.0000000000000563. [PubMed] [CrossRef] [Google Scholar]
75. Piplani H, et al. Simvastatin induces autophagic flux to restore cerulein-impaired phagosome-lysosome fusion in acute pancreatitis. Biochim Biophys Acta Mol Basis Dis. 2019; 1865 (11): 165530. doi: 10.1016/j.bbadis.2019.08.006. [PubMed] [CrossRef][Google Scholar]
76. Loera-Valencia R, et al. Alterations in cholesterol metabolism as a risk factor for developing Alzheimer's disease: Potential novel targets for treatment. J Steroid Biochem Mol Biol. 2019; 190:104-114. doi: 10.1016/j.jsbmb.2019.03.003. [PubMed] [CrossRef] [Google Scholar]
77. Mareninova O A, et al. Cell death in pancreatitis: caspases protect from necrotizing pancreatitis. J Biol Chem. 2006; 281 (6): 3370-3381. doi: 10.1074/jbc.M511276200. [PubMed] [CrossRef] [Google Scholar]
78. Tanaka Y, et al. Accumulation of autophagic vacuoles and cardiomyopathy in LAMP-2-deficient mice. Nature. 2000; 406 (6798): 902-906. doi: 10.1038/35022595. [PubMed] [CrossRef][Google Scholar]
79. Hashimoto D, et al. Involvement of autophagy in trypsinogen activation within the pancreatic acinar cells. J Cell Biol. 2008; 181 (7): 1065-1072. doi: 10.1083/jcb.200712156. [PMC free article] [PubMed] [CrossRef] [Google Scholar]
80. Liang T, et al. Ex vivo human pancreatic slice preparations offer a valuable model for studying pancreatic exocrine biology. J Biol Chem. 2017; 292 (14): 5957-5969. doi: 10.1074/jbc.M117.777433. [PMC free article] [PubMed] [CrossRef] [Google Scholar]
81. Lugea A, et al. Human pancreatic acinar cells: proteomic characterization, physiologic responses, and organellar disorders in ex vivo pancreatitis. Am J Pathol. 2017; 187 (12): 2726-2743. [PMC free article] [PubMed] [Google Scholar]

### EXAMPLE 1 REFERENCES IN METHODS

1. Mareninova O A, et al. Lysosome associated membrane proteins maintain pancreatic acinar cell homeostasis: LAMP-2 deficient mice develop pancreatitis. Cell Mol Gastroenterol Hepatol. 2015; 1 (6): 678-94.
2. Mareninova O A, et al. Transgenic expression of GFP-LC3 perturbs autophagy in exocrine pancreas and acute pancreatitis responses in mice. Autophagy. 2020; 16 (11): 2084-97.
3. Biczo G, et al. Mitochondrial dysfunction, through impaired autophagy, leads to endoplasmic reticulum stress, deregulated lipid metabolism, and pancreatitis in animal models. Gastroenterology. 2018; 154 (3): 689-703.
4. Mukherjee R, et al. Mechanism of mitochondrial permeability transition pore induction and damage in the pancreas: inhibition prevents acute pancreatitis by protecting production of ATP. Gut. 2016; 65 (8): 1333-46.
5. Shalbueva N, et al. Effects of oxidative alcohol metabolism on the mitochondrial permeability transition pore and necrosis in a mouse model of alcoholic pancreatitis. Gastroenterology. 2013; 144 (2): 437-46 e6.
6. Mareninova O A, et al. Cell death in pancreatitis: caspases protect from necrotizing pancreatitis. J Biol Chem. 2006; 281 (6): 3370-81.
7. Mareninova O A, et al. Impaired autophagic flux mediates acinar cell vacuole formation and trypsinogen activation in rodent models of acute pancreatitis. J Clin Invest. 2009; 119 (11): 3340-55.
8. Dong W, Vuletic S, and Albers J J. Differential effects of simvastatin and pravastatin on expression of Alzheimer's disease-related genes in human astrocytes and neuronal cells. J Lipid Res. 2009; 50 (10): 2095-102.
9. Ziros P, et al. Hepatic Fgf21 expression is repressed after simvastatin treatment in mice. PloS one. 2016; 11 (9): e0162024.
10. Heckmann B L, et al. Liver X receptor alpha mediates hepatic triglyceride accumulation through upregulation of G0/G1 Switch Gene 2 expression. JCI Insight. 2017; 2 (4): e88735.
11. Takano T, et al. Augmentation of DHCR24 expression by hepatitis C virus infection facilitates viral replication in hepatocytes. J Hepatol. 2011; 55 (3): 512-21.
12. Liang T, et al. Ex vivo human pancreatic slice preparations offer a valuable model for studying pancreatic exocrine biology. J Biol Chem. 2017; 292 (14): 5957-69.
13. Odinokova I V, et al. Mechanisms regulating cytochrome c release in pancreatic mitochondria. Gut. 2009; 58 (3): 431-42.
14. Barrientos A. In vivo and in organello assessment of OXPHOS activities. Methods. 2002; 26 (4): 307-16.
15. Maertin S, et al. Roles of autophagy and metabolism in pancreatic cancer cell adaptation to environmental challenges. Am J Physiol Gastrointest Liver Physiol 2017; 313: G524-G536. 16. Otani T, Chepilko S M, Grendell J H, and Gorelick F S. Codistribution of TAP and the granule membrane protein GRAMP-92 in rat caerulein-induced pancreatitis. Am J Physiol. 1998; 275 (5): G999-G1009.
17. Tuohetahuntila M, et al. Lysosome-mediated degradation of a distinct pool of lipid droplets during hepatic stellate cell activation. J Biol Chem. 2017; 292 (30): 12436-48.
18. Loregger A, Nelson J K, and Zelcer N. Assaying low-density-lipoprotein (LDL) uptake into cells. Methods Mol Biol. 2017; 1583:53-63.
19. Staples E, Ingram R J, Atherton J C, Robinson K. Optimising the quantification of cytokines present at low concentrations in small human mucosal tissue samples using Luminex assays. J Immunol Methods. 2013; 394 (1-2): 1-9.

### REFERENCES FOR EXAMPLE 2

1. Pandol S J, Saluja A K, Imrie C W, et al. Acute pancreatitis: bench to the bedside. Gastroenterology 2007; 132:1127-51. Erratum: ibid, 133:1056.
2. Peery A F, Crockett S D, Murphy C C, et al. Burden and cost of gastrointestinal, liver, and pancreatic diseases in the United States: Update 2018. Gastroenterology 2019; 156:254-272 e11.
3. Sadiq N, Gillani S W, Al Saeedy D, et al. Clinical review of acute, recurrent, and chronic pancreatitis: Recent updates of 2013-2019 literature. J Pharm Bioallied Sci 2020; 12:112-123.
4. Lerch M M, Gorelick F S. Models of acute and chronic pancreatitis. Gastroenterology 2013; 144:1180-93.
5. Gukovskaya A S, Gukovsky I. Autophagy and pancreatitis. Am J Physiol Gastrointest Liver Physiol 2012; 303: G993-G1003. PMC3517664
6. Gukovskaya A S, Gukovsky I, Algul H, et al. Autophagy, inflammation, and immune dysfunction in the pathogenesis of pancreatitis. Gastroenterology 2017; 153:1212-1226. PMC6338477
7 Gukovsky I, Li N, Todoric J, et al. Inflammation, autophagy, and obesity: common features in the pathogenesis of pancreatitis and pancreatic cancer. Gastroenterology 2013; 144:1199-209 e4. PMC3517664
8. Parzych K R, Klionsky D J. An overview of autophagy: morphology, mechanism, and regulation. Antioxid Redox Signal 2014; 20:460-73.
9 Gukovskaya A S, Gorelick F, Groblewski G E, et al. Recent insights into the pathogenic mechanism of pancreatitis: role of acinar cell organelle disorders. Pancreas 2019; 48:459-470. PMC6461375
10. Mareninova O A, Sendler M, Malla S R, et al. Lysosome associated membrane proteins maintain pancreatic acinar cell homeostasis: LAMP-2 deficient mice develop pancreatitis. Cell Mol Gastroenterol Hepatol 2015; 1:678-694. PMC4673685
11. Fortunato F, Burgers H, Bergmann F, et al. Impaired autolysosome formation correlates with Lamp-2 depletion: role of apoptosis, autophagy, and necrosis in pancreatitis. Gastroenterology 2009; 137:350-60, 360 el-5.
12. Wang S, Ni H M, Chao X, et al. Impaired TFEB-mediated lysosomal biogenesis promotes the development of pancreatitis in mice and is associated with human pancreatitis. Autophagy 2019; 15:1954-1969. PMC6844531.
13. Mareninova O A, Vegh E T, Shalbueva N, et al. Dysregulation of mannose-6-phosphate-dependent cholesterol homeostasis in acinar cells mediates pancreatitis. J Clin Invest 2021 Aug. 2; 131 (15): e146870. doi: 10.1172/JCI146870. PMC8321573.
14. Coutinho M F, Prata M J, Alves S. Mannose-6-phosphate pathway: a review on its role in lysosomal function and dysfunction. Mol Genet Metab 2012; 105:542-50.
15. Diakopoulos K N, Lesina M, Wormann S, et al. Impaired autophagy induces chronic atrophic pancreatitis in mice via sex- and nutrition-dependent processes. Gastroenterology 2015; 148:626-638 e17.
16. Antonucci L, Fagman J B, Kim J Y, et al. Basal autophagy maintains pancreatic acinar cell homeostasis and protein synthesis and prevents ER stress. Proc Natl Acad Sci USA 2015; 112: E6166-74.
17. Biczo G, Vegh E T, Shalbueva N, et al. Mitochondrial dysfunction, through impaired autophagy, leads to endoplasmic reticulum stress, deregulated lipid metabolism, and pancreatitis in animal models. Gastroenterology 2018; 154:689-703. PMC6369139.
18. Ikonen E. Cellular cholesterol trafficking and compartmentalization. Nat Rev Mol Cell Biol 2008; 9:125-38.
19. Korber M, Klein I, Daum G. Steryl ester synthesis, storage and hydrolysis: A contribution to sterol homeostasis. Biochim Biophys Acta Mol Cell Biol Lipids 2017; 1862:1534-1545.
20. Mesmin B, Antonny B, Drin G. Insights into the mechanisms of sterol transport between organelles. Cell Mol Life Sci 2013; 70:3405-21.
21. Thelen A M, Zoncu R. Emerging roles for the lysosome in lipid metabolism. Trends Cell Biol 2017; 27:833-850.
22. Burke A C, Huff M W. ATP-citrate lyase: genetics, molecular biology and therapeutic target for dyslipidemia. Curr Opin Lipidol 2017; 28:193-200.
23. Verberk S GS, Kuiper K L, Lauterbach M A, et al. The multifaceted therapeutic value of targeting ATP-citrate lyase in atherosclerosis. Trends Mol Med 2021; 27:1095-1105.
24. Maxfield F R, Tabas I. Role of cholesterol and lipid organization in disease. Nature 2005; 438:612-21.
25. Dai L, Zou L, Meng L, et al. Cholesterol metabolism in neurodegenerative diseases: Molecular mechanisms and therapeutic targets. Mol Neurobiol 2021; 58:2183-2201.
26. Vanier M T. Complex lipid trafficking in Niemann-Pick disease type C. J Inherit Metab Dis 2015; 38:187-99.
27. Lusis A J. Atherosclerosis. Nature 2000; 407:233-41.
28. Goldstein J L, Brown M S. A century of cholesterol and coronaries: from plaques to genes to statins. Cell 2015; 161:161-172.
29. Cluzeau C V, Watkins-Chow D E, Fu R, et al. Microarray expression analysis and identification of serum biomarkers for Niemann-Pick disease, type C1. Hum Mol Genet 2012; 21:3632-46.
30. Vanier M T, Latour P. Laboratory diagnosis of Niemann-Pick disease type C: the filipin staining test. Methods Cell Biol 2015; 126:357-75.
31. Loera-Valencia R, Goikolea J, Parrado-Fernandez C, et al. Alterations in cholesterol metabolism as a risk factor for developing Alzheimer's disease: Potential novel targets for treatment. J Steroid Biochem Mol Biol 2019; 190:104-114.
32. Li J J, Liu H H, Wu N Q, et al. Statin intolerance: an updated, narrative review mainly focusing on muscle adverse effects. Expert Opin Drug Metab Toxicol 2020; 16:837-851.
33. Alam U, Al-Bazz D Y, Soran H. Bempedoic Acid: The new kid on the block for the treatment of dyslipidemia and LDL cholesterol: a narrative review. Diabetes Ther 2021; 12:1779-1789.
34. Thisted H, Jacobsen J, Munk E M, et al. Statins and the risk of acute pancreatitis: a population-based case-control study. Aliment Pharmacol Ther 2006; 23:185-90.
35. Preiss D, Tikkanen M J, Welsh P, et al. Lipid-modifying therapies and risk of pancreatitis: a meta-analysis. JAMA 2012; 308:804-11.
36. Gornik I, Gasparovic V, Gubarev Vrdoljak N, et al. Prior statin therapy is associated with milder course and better outcome in acute pancreatitis—a cohort study. Pancreatology 2013; 13:196-200.
37. Wu B U, Pandol S J, Liu I L. Simvastatin is associated with reduced risk of acute pancreatitis: findings from a regional integrated healthcare system. Gut 2015; 64:133-8.
38. Lee P J, Modha K, Chua T, et al. Association of statins with decreased acute pancreatitis severity: a propensity score analysis. J Clin Gastroenterol 2018; 52:742-746.
39. Machicado J D, Papachristou G I. Pharmacologic management and prevention of acute pancreatitis. Curr Opin Gastroenterol 2019; 35:460-467.
40. de-Madaria E. Statins for the prevention of acute pancreatitis. Am J Gastroenterol 2017; 112:1765-1767.
41. Poropat G, Archibugi L, Korpela T, et al. Statin use is not associated with an increased risk of acute pancreatitis-A meta-analysis of observational studies. United European Gastroenterol J 2018; 6:1206-1214.
42. Apte M V, Pirola R C, Wilson J S. Mechanisms of alcoholic pancreatitis. J Gastroenterol Hepatol 2010; 25:1816-26.
43. Bansal P, Sonnenberg A. Pancreatitis is a risk factor for pancreatic cancer. Gastroenterology 1995; 109:247-51.
44. Williams V F, Ying S, Stahlman S. Acute and chronic pancreatitis, active component, U.S. Armed Forces, 2004-2018. Medical Surveillance Monthly Report (MSMR) 2020; 27:9-19.
45. Pushpakom S, Iorio F, Eyers P A, et al. Drug repurposing: progress, challenges and recommendations. Nat Rev Drug Discov 2019; 18:41-58.
46. Stancu C, Sima A. Statins: mechanism of action and effects. J Cell Mol Med 2001; 5:378-87.
47. Mareninova O A, Hermann K, French S W, et al. Impaired autophagic flux mediates acinar cell vacuole formation and trypsinogen activation in rodent models of acute pancreatitis. J Clin Invest 2009; 119:3340-55. PMC2769194
48. Li N, Wu X, Holzer R G, et al. Loss of acinar cell IKKalpha triggers spontaneous pancreatitis in mice. J Clin Invest 2013; 123:2231-43. PMC3635720
49. Mareninova O A, Jia W, Gretler S R, et al. Transgenic expression of GFP-LC3 perturbs autophagy in exocrine pancreas and acute pancreatitis responses in mice. Autophagy 2020; 16:2084-2097. PMC7595606
50. Maxfield F R, Wustner D. Analysis of cholesterol trafficking with fluorescent probes. Methods Cell Biol 2012; 108:367-93.
51. Carrer A, Trefely S, Zhao S, et al. Acetyl-CoA metabolism supports multistep pancreatic tumorigenesis. Cancer Discov 2019; 9:416-435.

1. A method of treating pancreatitis in a subject comprising administering to the subject a therapeutically effective amount of a cholesterol lowering drug.

2. The method of claim 1, wherein the cholesterol lowering drug is a statin.

3. The method of claim 2, wherein the statin is simvastatin.

4. The method of claim 1, wherein the cholesterol lowering drug is an adenosine triphosphate citrate lyase inhibitor.

5. The method of claim 1, wherein the adenosine triphosphate citrate lyase inhibitor is bempedoic acid.

6. The method of claim 1, wherein the cholesterol lowering drug normalizes cholesterol metabolism.

7. The method of claim 1, further comprising administering a second pancreatitis therapy to the subject.

8. The method of claim 7, wherein the second pancreatitis therapy is a pain medication, vitamins, a digestive, or i.v. fluids.

9. A method of reducing acinar cell cholesterol in a subject comprising administering to the subject a therapeutically effective amount of a cholesterol lowering drug.

10. The method of claim 9, wherein acinar cell cholesterol is reduced at least 10% when compared to untreated subjects.

11. A method of reducing necrosis in the pancreas of a subject comprising administering to the subject a therapeutically effective amount of a cholesterol lowering drug.

12. The method of claim 11, wherein the necrosis is due to pancreatitis

13. The method of claim 11, wherein the cholesterol lowering drug is a statin.

14. The method of claim 13, wherein the statin is simvastatin.

15. The method of claim 11, wherein the cholesterol lowering drug is an adenosine triphosphate citrate lyase inhibitor.

16. The method of claim 15, wherein the adenosine triphosphate citrate lyase inhibitor is bempedoic acid.

17. The method of claim 13, wherein the cholesterol lowering drug normalizes cholesterol metabolism.

18. The method of claim 1, wherein the cholesterol lowering drug reduce pancreatitis-induced increases in total cholesterol and trypsin activity in the subject.

19. The method of claim 1, wherein the cholesterol lowering drug reduces accumulation of LC3-II, p62, and/or ubiquitylated proteins in the subject.

20. The method of claim 1, wherein the cholesterol lowering drug normalizes mitochondrial fragmentation or abrogates CER-induced increase in MLN64 in the subject.
